(19) United States
(12) Patent Application Publication
(10) Pub. No.: US 2022/0192215 A1
(43) Pub. Date: Jun. 23, 2022

(54) ACIDIC BETA-LACTOGLOBULIN BEVERAGE PREPARATION

(71) Applicant: ARLA FOODS AMBA, Viby J (DK)

(72) Inventors: Søren Bang NIELSEN, Herning (DK); Kasper Bøgelund LAURIDSEN, Silkeborg (DK); Tanja Christine JÆGER, Kibæk (DK)

(21) Appl. No.: 17/254,759

(22) PCT Filed: Jun. 27, 2018
(86) PCT No.: PCT/EP2018/067299

§ 371 Date: Dec. 21, 2020

(51) Int. Cl. A23C 9/142; A23J 1/20; A23J 3/08; A23L 2/60; A23L 2/66; A23L 2/68; A23P 10/40; A23L 33/19

(52) U.S. Cl. CPC A23C 9/1427; A23J 1/205; A23J 3/08; A23L 33/19; A23L 2/66; A23L 2/68; A23P 10/40; A23L 2/60

(57) ABSTRACT

The present invention pertains to a new packaged, heat-treated beverage preparation having a pH in the range of 2.0-4.7. The invention furthermore relates to a method of producing a packaged, heat-treated beverage preparation and to different uses of the packaged heat-treated beverage preparation.

### FIELD OF THE INVENTION

The present invention pertains to a new packaged, heat-treated beverage preparation having a pH in the range of 2.0-4.7. The invention furthermore relates to a method of producing a packaged, heat-treated beverage preparation and to different uses of the packaged heat-treated beverage preparation.

### BACKGROUND

Nutritional supplements comprising whey proteins are commonly used for muscle synthesis, for weight control and for maintaining muscle and bodyweight. Nutritional supplements are targeted towards different kinds of consumers, e.g. sportsmen/women, athletes, children, elderly people and patients with or at risk of malnutrition, and/or with increased protein needs. Whey proteins can be isolated from milk serum or whey. Whey typically comprises a mixture of beta-lactoglobulin (BLG), alpha-lactalbumin (ALA), serum albumin and immunoglobulins, of which BLG is the most dominant. Whey protein concentrates (WPC) thus comprise a mixture of these proteins. Whey protein isolates (WPI) contain less fat and lactose than WPC.

Beverages comprising whey proteins are well known. For example acidic heat-treated beverages comprising whey proteins.

Etzel 2004 (Etzel, M. R., 2004, Manufacture and use of dairy protein fraction. American Society for Nutritional Science, pp. 996-1002) describes a beverage containing 2.5 wt % WPI at pH 2-7. They found that beverages that had been subjected to a thermal processing could only be obtained if an antiaggregant was added.

### SUMMARY OF THE INVENTION

The present inventors have observed that organoleptic characteristics such as astringency and mouthfeel play a significant role in the selection of liquid nutritional beverages by consumers. Some of the challenges in incorporating whey proteins in acidic heat-treated beverages are formation of unstable precipitate that sediment in the beverage, high viscosity or even gel-formation, and unpleasant taste due to high degree of astringency and/or a drying mouthfeeling.

An object of the present invention is to provide an acidic, packaged, heat-treated beverage preparation comprising whey protein and having improved organoleptic and/or visual properties.

Another object of the invention is to provide a high protein beverage with a low viscosity, a pleasant taste, optionally with low astringency, and which may either be transparent or opaque.

The present inventors have now discovered that such packaged, heat-treated beverages can be provided within a broad acidic pH range up to and including pH 4.7, while still having a low viscosity and optionally also a low level of astringency and drying mouthfeel. The invention provides both beverages that are transparent and beverages that are opaque but stable.

Thus, an aspect of the invention pertains to a packaged, heat-treated beverage preparation having a pH in the range of 2.0-4.7, the beverage comprising
a total amount of protein of 2 to 45% w/w relative to the weight of the beverage, wherein at least 85% w/w of the protein is BLG, and
optionally, sweetener and/or flavour.

Another aspect of the invention pertains to a method of producing a packaged, heat-treated beverage preparation having a pH in the range of 2.0-4.7, comprising the following steps:

a) Providing a liquid solution comprising:
a total amount of protein of 2 to 45% by weight, wherein at least 85% of the protein is BLG
optionally, sweetener and/or flavour

b) packaging the liquid solution,

wherein the liquid solution of step a) and/or the packaged liquid solution of step b) is subjected to a heat-treatment comprising at least pasteurisation.

Yet an aspect of the invention pertains to use of a protein solution comprising a total amount of protein of 2 to 45% w/w relative to the weight of the solution, wherein at least 85 w/w % of the protein is BLG for controlling the turbidity of a heat-treated acidic beverage preparation having a pH in the range of 2.0-4.7.

Still another aspect of the invention pertains to use of a protein solution comprising a total amount of protein of 2 to 45% w/w relative to the weight of the solution, wherein at least 85 w/w % of the protein is BLG for controlling the astringency of a heat-treated acidic beverage preparation having a pH in the range of 2.0-4.7.

A further aspect of the invention pertains to a packaged heat-treated beverage preparation according to the invention for use in a method for the treatment of diseases associated with protein malabsorption.

A further aspect of the invention pertains to use of a packaged heat-treated beverage preparation according to the invention as a dietary supplement.

### BRIEF DESCRIPTION OF THE FIGURES

FIG. 1 shows images of BLG and WPI beverages having a pH of 3.7 and a protein content of 6% w/w, heat-treated at 120° C. for 20 seconds and 75° C. for 15 seconds.

FIG. 2 shows images of WPI-B pH 3.0-3.7 120° C. and BLG pH 3.7 120° C./20 s.

FIG. 3 shows images of WPI-B pH 3.0-3.7 75° C. and BLG pH 3.7 at 75° C./15 seconds.

FIG. 4 shows images of WPI-B pH 3.7 and BLG pH 3.9, 75° C./15 seconds.

FIG. 5 illustrates turbidity of 6% UHT treated (120° C./20 s) BLG beverage preparation.

FIG. 6 illustrates turbidity of 6% pasteurized (75° C./15 s) BLG beverage composition.

FIG. 7 illustrates viscosity of a 6% UHT treated (120° C./20 s) BLG beverage preparation.

FIG. 8 illustrates yellowness (b*) of 6% UHT treated (120° C./20 s) beverage compositions FIG. 9 illustrates yellowness (b*) of 6% pasteurized (75° C./15 s) beverage compositions FIG. 10 shows images of 15% BLG beverage pH 3.7 (left) and 6% WPI-a pH 3.7 (right), at 75 C/15 sec.

FIG. 11 shows sensory evaluation of high protein BLG beverage compositions and images of 6 w/w % and 15 w/w % BLG samples at pH3.7.

FIG. 12 shows high protein beverage preparations prepared by heating of (left to right) 30, 27.5, 25, 20% BLG at 75° C. for 5 minutes. Viscosity remained low even after heating.

FIG. 13 shows images of different WPI and BLG samples.

FIG. 14 shows sensory evaluation on beverages (scale from 0 to 15). WPI pH 3.0 120° C./20 s and BLG pH 3.7 75° C./15 sec.

FIG. 15 demonstrates the effect of pH and temperature on acid taste.

FIG. 16 shows sensory data on astringency of BLG beverages at pH 3.0 (120° C./20 sec) and pH 3.7 (75° C./15 sec).

FIG. 17 shows sensory data on drying mouthfeel of BLG pH 3.7 beverages at 120° C./20 sec and 75 C°/15 sec.

FIG. 18 shows sensory data on whey aroma when BLG is kept in native conformation.

FIG. 19 shows images of 6% BLG beverages heat-treated at 95° C. for 5 min, pH 3.7 and minerals added.

FIG. 20 shows images of 6% BLG beverages pH 3.7, heat-treated at 75° C. for 5 min. and minerals added.

FIG. 21 illustrates stability of milky BLG beverages, pH 4.3, with and without sucrose, heat-treated at 93° C. for 4 minutes.

FIG. 22 shows images of opaque 6% protein BLG beverages prepared by 75° C./5 min. heating at pH 4.2-4.5.

FIG. 23 shows images of BLG and SPI beverages at pH 3.7, heat-treated at 75° C. for 5 min.

FIG. 24 shows images of BLG and SPI beverages at pH 3.7.

### DETAILED DESCRIPTION

### Definitions

In the context of the present invention, the term “beta-lactoglobulin” or “BLG” pertains to beta-lactoglobulin from mammal species, e.g. in native, unfolded and/or glycosylated forms and includes the naturally occurring genetic variants. The term furthermore includes aggregated BLG, precipitated BLG and crystalline BLG. When referring to the amount of BLG reference is made to the total amount of BLG including aggregated BLG. The total amount of BLG is determined according to Example 1.31. The term “aggregated BLG” pertains to BLG which is at least partially unfolded and which furthermore has aggregated with other denatured BLG molecules and/or other denatured whey proteins, typically by means of hydrophobic interactions and/or covalent bonds.

BLG is the most predominant protein in bovine whey and milk serum and exists in several genetic variants, the main ones in cow milk being labelled A and B. BLG is a lipocalin protein, and can bind many hydrophobic molecules, suggesting a role in their transport. BLG has also been shown to be able to bind iron via siderophores and might have a role in combating pathogens. A homologue of BLG is lacking in human breast milk.

Bovine BLG is a relatively small protein of approx. 162 amino acid residues with a molecular weight of approx. 18.3-18.4 kDa. Under physiological conditions, it is predominantly dimeric, but dissociates to a monomer below about pH 3, preserving its native state as determined using Nuclear Magnetic Resonance spectroscopy. Conversely, BLG also occurs in tetrameric, octameric and other multimeric aggregation forms under a variety of natural conditions.

In the context of the present invention, the term “non-aggregated beta-lactoglobulin” or “non-aggregated BLG” also pertains to beta-lactoglobulin from mammal species, e.g. in native, unfolded and/or glycosylated forms and includes the naturally occurring genetic variants. However, the term does not include aggregated BLG, precipitated BLG or crystallised BLG. The amount or concentration of non-aggregated BLG is determined according to Example 1.6.

The percentage of non-aggregated BLG relative to total BLG is determined by calculate (m_{total BLG}−m_{non-aggregate BLG})/m_{total BLG}*100%. m_{total BLG} is the concentration or amount of BLG determined according to Example 1.31 and m_{non-aggregated BLG} is the concentration or amount of non-aggregated BLG determined according to Example 1.6.

In the context of the present invention, the term “crystal” pertains to a solid material whose constituents (such as atoms, molecules or ions) are arranged in a highly ordered microscopic structure, forming a crystal lattice that extends in all directions.

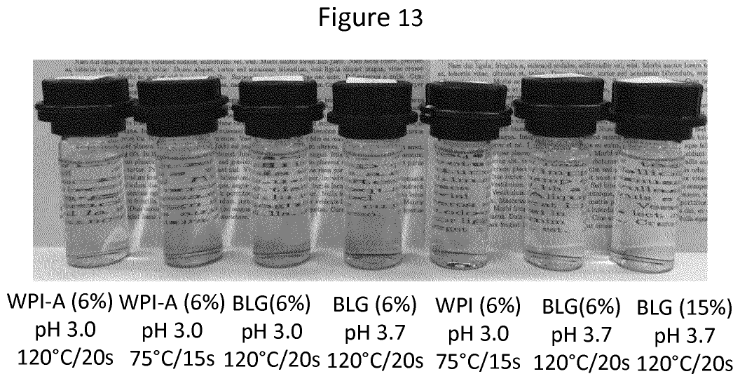

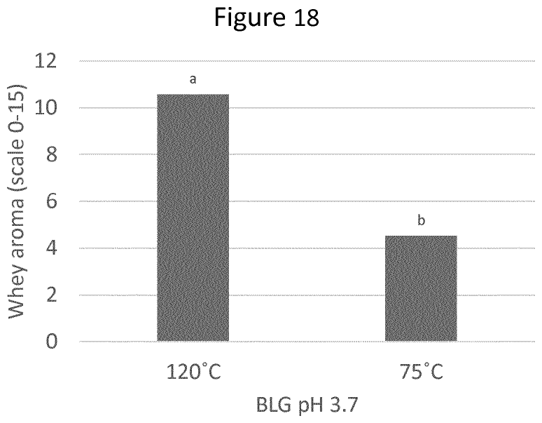

In the context of the present invention, the term “BLG crystal” pertains to protein crystals that primarily contain non-aggregated and preferably native BLG arranged in a highly ordered microscopic structure, forming a crystal lattice that extends in all directions. The BLG crystals may e.g. be monolithic or polycrystalline and may e.g. be intact crystals, fragments of crystals, or a combination thereof. Fragments of crystal are e.g. formed when intact crystals are subjected to mechanical shear during processing. Fragments of crystals also have the highly ordered microscopic structure of crystal but may lack the even surface and/or even edges or corners of an intact crystal. See e.g. FIG. 18 of PCT application no. PCT/EP2017/084553 for an example of many intact BLG crystals and FIG. 13 PCT application no. PCT/EP2017/084553 for an example of fragments of BLG crystals. In both cases, the BLG crystal or crystal fragments can be identified visually as well-defined, compact and coherent structures using light microscopy. BLG crystal or crystal fragments are often at least partially transparent. Protein crystals are furthermore known to be birefringent and this optical property can be used to identify unknown particles having a crystal structure. Non-crystalline BLG aggregates, on the other hand, often appear as poorly defined, non-transparent, and as open or porous lumps of irregular size.

In the context of the present invention, the term “crystallise” pertains to the formation of protein crystals. Crystallisation may e.g. happen spontaneously or be initiated by the addition of crystallisation seeds.

In the context of the present invention, the term “edible composition” pertains to a composition that is safe for human consumption and use as a food ingredient and that does not contain problematic amounts of toxic components, such as toluene or other unwanted organic solvents.

In the context of the present invention, the term “ALA” or “alpha-lactalbumin” pertains to alpha-lactalbumin from mammal species, e.g. in native and/or glycosylated forms and includes the naturally occurring genetic variants. The term furthermore includes aggregated ALA and precipitated BLG. When referring to the amount of ALA reference is made to the total amount of ALA including e.g. aggregated ALA. The total amount of ALA is determined according to Example 1.31. The term “aggregated ALA” pertains to ALA which typically is at least partially unfolded and which furthermore has aggregated with other denatured ALA molecules and/or other denatured whey proteins, typically by means of hydrophobic interactions and/or covalent bonds.

Alpha-lactalbumin (ALA) is a protein present in the milk of almost all mammalian species. ALA forms the regulatory subunit of the lactose synthase (LS) heterodimer and β-1,4-galactosyltransferase (beta4Gal-T1) forms the catalytic component. Together, these proteins enable LS to produce lactose by transferring galactose moieties to glucose. One of the main structural differences with beta-lactoglobulin is that ALA does not have any free thiol group that can serve as the starting-point for a covalent aggregation reaction.

In the context of the present invention, the term “non-aggregated ALA” also pertains to ALA from mammal species, e.g. in native, unfolded and/or glycosylated forms and includes the naturally occurring genetic variants. However, the term does not include aggregated ALA or precipitated ALA. The amount or concentration of non-aggregated BLG is determined according to Example 1.6.

The percentage of non-aggregated ALA relative to total ALA is determined by calculate (m_{total ALA}−m_{non-aggregate ALA})/m_{total ALA}*100%. m_{total ALA} is the concentration or amount of ALA determined according to Example 1.31 and m_{non-aggregated ALA} is the concentration or amount of non-aggregated ALA determined according to Example 1.6.

In the context of the present invention, the term “caseinomacropeptide” or “CMP” pertains to the hydrophilic peptide, residue 106-169, originated from the hydrolysis of “K-CN” or “kappa-casein” from mammal species, e.g. in native and/or glycosylated forms and includes the naturally occurring genetic variants, by an aspartic proteinase, e.g. chymosin.

In the context of the present invention, the term “BLG isolate” means a composition that contains BLG in an amount of at least 85% w/w relative to total protein. A BLG isolate preferably has a total protein content of a least 30% w/w, and preferably at least 80% w/w relative to total solids.

In the context of the present invention, the term “BLG isolate powder” pertains to a BLG isolate in powder form and preferably a free-flowing powder.

In the context of the present invention, the term “BLG isolate liquid” pertains to a BLG isolate in liquid form and preferably an aqueous liquid.

The term “whey” pertains to the liquid phase that is left after the casein of milk has been precipitated and removed. Casein precipitation may e.g. be accomplished by acidification of milk and/or by use of rennet enzyme. Several types of whey exist, such as “sweet whey”, which is the whey product produced by rennet-based precipitation of casein, and “acid whey” or “sour whey”, which is the whey product produced by acid-based precipitation of casein. Acid-based precipitation of casein may e.g. be accomplished by addition of food acids or by means of bacterial cultures.

The term “milk serum” pertains to the liquid which remains when casein and milk fat globules have been removed from milk, e.g. by microfiltration or large pore ultrafiltration. Milk serum may also be referred to as “ideal whey”.

The term “milk serum protein” or “serum protein” pertains to the protein which is present in the milk serum.

In the context of the present invention, the term “whey protein” pertains to protein that is found in whey or in milk serum. Whey protein may be a subset of the protein species found in whey or milk serum, and even a single whey protein species or it may be the complete set of protein species found in whey or/and in milk serum.

In the context of the present invention, the main non-BLG proteins of a standard whey protein concentrate from sweet whey are ALA, CMP, bovine serum albumin, immunoglobulin, osteopontin, lactoferrin, and lactoperoxidase. In the context of the present invention, the weight percentages of the main non-BLG whey proteins of a standard whey protein concentrate from sweet whey are:

ALA in an amount of 18% w/w relative to total protein,

CMP in an amount of 18% w/w relative to total protein,

BSA in an amount of 4% w/w relative to total protein,

Casein species in an amount of 5% w/w relative to total protein,

Immunoglobulin in an amount of 6% w/w relative to total protein,

Osteopontin in an amount of 0.5% w/w relative to total protein,

Lactoferrin in an amount of 0.1% w/w relative to total protein, and

Lactoperoxidase in an amount of 0.1% w/w relative to total protein.

In the context of the present invention the term “mother liquor” pertains to the whey protein solution that remains after BLG has been crystallised and the BLG crystals have be at least partially removed. The mother liquor may still contain some BLG crystals but normally only small BLG crystals that have escaped the separation.

In the context of the present invention, the term casein pertains to casein protein found in milk and encompasses both native micellar casein as found in raw milk, the individual casein species, and caseinates.

In the context of the present invention, a liquid which is “supersaturated” or “supersaturated with respect to BLG” contains a concentration of dissolved, non-aggregated BLG which is above the saturation point of non-aggregated BLG in that liquid at the given physical and chemical conditions. The term “supersaturated” is well-known in the field of crystallisation (see e.g. Gérand Coquerela, “Crystallization of molecular systems from solution: phase diagrams, supersaturation and other basic concepts”, Chemical Society Reviews, p. 2286-2300, Issue 7, 2014) and supersaturation can be determined by a number of different measurement techniques (e.g. by spectroscopy or particle size analysis). In the context of the present invention, supersaturation with respect to BLG is determined by the following procedure.

Procedure for Testing Whether a Liquid at a Specific Set of Conditions is Supersaturated with Respect to BLG:

a) Transfer a 50 ml sample of the liquid to be tested to a centrifuge tube (VWR Catalogue no. 525-0402) having a height of 115 mm, an inside diameter of 25 mm and a capacity of 50 mL. Care should be taken to keep the sample and subsequent fractions thereof at the original physical and chemical conditions of the liquid during steps a)-h).

b) The sample is immediately centrifuged at 3000 g for 3.0 minutes with max. 30 seconds acceleration and max 30 seconds deceleration.

c) Immediately after the centrifugation, transfer as much as possible of the supernatant (without disturbing the pellet if a pellet has formed) to a second centrifuge tube (same type as in step a)

d) Take a 0.05 mL subsample of the supernatant (subsample A)

e) Add 10 mg of BLG crystals (at least 98% pure, non-aggregated BLG relative to total solids) having a particle size of at most 200 micron to a second centrifuge tube and agitate the mixture.

f) Allow the second centrifuge tube to stand for 60 minutes at the original temperature.

g) Immediately after step f), centrifuge the second centrifuge tube at 500 g for 10 minutes and then take another 0.05 mL subsample of the supernatant (subsample B).

h) Recover the centrifugation pellet of step g) if there is one, resuspend it in milliQ water and immediately inspect the suspension for presence of crystals that are visible by microscopy.

i) Determine the concentration of non-aggregated BLG in subsamples A and B using the method outlined in Example 1.6—the results are expressed as % BLG w/w relative to the total weight of the subsamples. The concentration of non-aggregated BLG of subsample A is referred to as C_{BLG, A}, and the concentration of non-aggregated BLG of subsample B is referred to as C_{BLG, B}.

j) The liquid from which the sample of step a) was taken was supersaturated (at the specific conditions) if c_{BLG, B} is lower than c_{BLG, A} and if crystals are observed in step i).

In the context of the present invention, the terms “liquid” and “solution” encompass both compositions that are free of particulate matter and compositions that contain a combination of liquid and solid and/or semi-solid particles, such as e.g. protein crystals or other protein particles. A “liquid” or a “solution” may therefore be a suspension or even a slurry. However, a “liquid” and “solution” are preferably pumpable.

In the context of the present invention, the terms “whey protein concentrate” (WPC) and “serum protein concentrate” (SPC) pertain to dry or aqueous compositions which contain a total amount of protein of 20-89% w/w relative to total solids.

A WPC or an SPC preferably contains:

20-89% w/w protein relative to total solids,

15-70% w/w BLG relative to total protein,

8-50% w/w ALA relative to total protein, and

0-40% w/w CMP relative to protein.

Alternatively, but also preferred, a WPC or an SPC may contain:

20-89% w/w protein relative to total solids,

15-90% w/w BLG relative to total protein,

4-50% w/w ALA relative to total protein, and

0-40% w/w CMP relative to protein.

Preferably, a WPC or an SPC contains:

20-89% w/w protein relative to total solids,

15-80% w/w BLG relative to total protein,

4-50% w/w ALA relative to total protein, and

0-40% w/w CMP relative to protein.

More preferably a WPC or an SPC contains:

70-89% w/w protein relative to total solids,

30-90% w/w BLG relative to total protein,

4-35% w/w ALA relative to total protein, and

0-25% w/w CMP relative to protein.

SPC typically contain no CMP or only traces of CMP.

The terms “whey protein isolate” (WPI) and “serum protein isolate” (SPI) pertain to dry or aqueous compositions which contain a total amount of protein of 90-100% w/w relative to total solids.

A WPI or an SPI preferably contains:

90-100% w/w protein relative to total solids,

15-70% w/w BLG relative to total protein,

8-50% w/w ALA relative to total protein, and

0-40% w/w CMP relative to total protein.

Alternatively, but also preferred, a WPI or an SPI may contain:

90-100% w/w protein relative to total solids,

30-95% w/w BLG relative to total protein,

4-35% w/w ALA relative to total protein, and

0-25% w/w CMP relative to total protein.

More preferably a WPI or an SPI may contain:

90-100% w/w protein relative to total solids,

30-90% w/w BLG relative to total protein,

4-35% w/w ALA relative to total protein, and

0-25% w/w CMP relative to total protein.

SPI typically contain no CMP or only traces of CMP.

In the context of the present invention, the term “additional protein” means a protein that is not BLG. The additional protein that is present in the whey protein solution typically comprises one or more of the non-BLG proteins that are found in milk serum or whey. Non-limiting examples of such proteins are alpha-lactalbumin, bovine serum albumin, immunoglobulines, caseinomacropeptide (CMP), osteopontin, lactoferrin, and milk fat globule membrane proteins.

The terms “consists essentially of” and “consisting essentially of” mean that the claim or feature in question encompasses the specified materials or steps and those that do not materially affect the basic and novel characteristic(s) of the claimed invention.

In the context of the present invention, the phrase “Y and/or X” means “Y” or “X” or “Y and X”. Along the same line of logic, the phrase “n₁, n₂, . . . , nᵢ₋₁, and/or nᵢ” means “n₁” or “n₂” or . . . or “nᵢ₋₁” or “nᵢ” or any combination of the components: n₁, n₂, . . . nᵢ₋₁, and nᵢ.

In the context of the present invention, the term “dry” or “dried” means that the composition or product in question comprises at most 10% w/w water, preferably at most 6% w/w and more preferably even less.

In the context of the present invention, the term “physical microbial reduction” pertains to physical interaction with a composition which results in reduction of the total amount of viable microorganisms of the composition. The term does not encompass addition of chemicals that result in killing of microorganisms. The term furthermore does not encompass the heat exposure to which the atomized droplets of liquid are exposed to during spray-drying but include possible pre-heating prior to spray-drying.

In the context of the present invention, the pH of a powder refers to the pH of 10 g of the powder mixed into 90 g demineralised water and is measured according to Example 1.16.

In the context of the present invention, the weight percentage (% w/w) of a component of a certain composition, product, or material means the weight percentage of that component relative to the weight of the specific composition, product, or material unless another reference (e.g total solids or total protein) is specifically mentioned.

In the context of the present invention, the process step “concentration” and the verb “concentrate” pertain to concentration of protein and encompass both concentration of protein on total solids basis and concentration of protein on a total weight basis. This means e.g. that concentration does not necessarily require that the absolute concentration w/w of protein of a composition increases as long at the content of protein increases relative to total solids.

In the context of the present invention, the term “weight ratio” between component X and component Y means the value obtained by the calculation mx/my wherein mx is the amount (weight) of components X and my is the amount (weight) of components Y.

In the context of the present invention, the term “at least pasteurisation” pertains to a heat-treatment which has microbial killing effect equal to or higher than a heat-treatment of 70 degrees C. for 10 seconds. The reference for determining the bacteria killing effect is E. coli O157:H7.

In the context of the present invention, the term “whey protein feed” pertains to whey protein source from which the liquid BLG isolate is derived. The whey protein feed has a lower content of BLG relative to total protein than the liquid BLG isolate and is typically a WPC, a WPI, an SPC or an SPI.

In the context of the present invention, the term “BLG-enriched composition” pertains to the BLG-enriched composition resulting from isolating BLG from the whey protein feed. The BLG-enriched composition typically comprises the same whey proteins as the whey protein feed but BLG is present in significantly higher concentration relative to total protein than in whey protein feed. The BLG-enriched composition may e.g. be prepared from the whey protein feed by chromatography, protein crystallisation and/or membrane-based protein fractionation. The BLG-enriched composition comprises BLG in an amount of at least 85% w/w relative to total protein, and preferably at least 90% w/w. In some cases the BLG-enriched composition can be used directly as the liquid BLG isolate. However, often additional processing is required to convert the BLG-enriched composition to the liquid BLG isolate.

In the context of the present invention, the term “whey protein solution” is used to describe the special aqueous whey protein composition that is supersaturated with respect to BLG in salting-in mode and useful for preparing BLG crystals.

In the context of the present invention, the term “sterile” means that the sterile composition or product in question does not contain any viable microorganisms and therefore is devoid of microbial growth during storage at room temperature. A composition that has been sterilised is sterile.

When a liquid, such as a beverage preparation, is sterilized and packaged aseptically in a sterile container it typically has a shelf life of at least six months at room temperature. The sterilization treatment kills spores and microorganisms that could cause spoilage of the liquid.

In the context of the present invention the term “energy content” means the total content of energy contained in a food product. The energy content can be measured in kilojoule (kJ) or kilo calories (kcal) and are referred to as calories per amount of food product, e.g. kcal per 100 gram of the food product. One example is a beverage having an energy content of 350 kcal/100 gram of the beverage.

The total energy content of a food product includes the energy contribution from all the macronutrients present in the food product, e.g. energy from protein, lipid and carbohydrate. The distribution of energy from the macronutrients in the food product can be calculated based on the amount of the macronutrients in the food product and the contribution of the macronutrient to the total energy content of the food product. The energy distribution can be stated as energy percent (E %) of the total energy content of the food product. For example for a beverage comprising 20 E % protein, 50 E % carbohydrate and 30 E % lipid, this means that 20% of the total energy comes from protein, 50% of the total energy comes from carbohydrate and 30% of the total energy comes from fat (lipid).

In the context of the present invention the term “nutritionally complete nutritional supplement” is understood as a food product comprising protein, lipid and carbohydrate and further comprising vitamins, minerals and trace elements, where the beverage has a nutrient profile matching a complete and healthy diet.

In the context of the present invention the term “nutritionally incomplete supplement” means food products comprising one or more macro nutrients and optionally further comprising vitamins, minerals and trace elements. A nutritionally incomplete beverage may comprise protein as the only nutrient or may for example comprise protein and a carbohydrate.

The term “food for special medical purposes (FSMP)” or “medical food” are food products for oral ingestion or tube feeding, which are used for specific medical disorders, diseases or conditions for which there are distinctive nutritional requirements and which are used under medical supervision. A medical food can be a nutritionally complete supplement/beverage or an nutritionally incomplete supplement/beverage.

The term “nutrient” means a substance used by an organism to survive, grow and reproduce. Nutrients can be either macronutrients or micronutrients. Macronutrients are nutrients that provide energy when consumed e.g. protein, lipid and carbohydrate. Micronutrients are nutrients like vitamins, minerals and trace elements.

The term “nutrient” means a substance used by an organism to survive, grow and reproduce. Nutrients can be either macronutrients or micronutrients. Macronutrients are nutrient that provide energy when consumed e.g. protein, lipid and carbohydrate. Micronutrients are nutrients are vitamins, minerals and trace elements.

By the term “instant beverage powder” or “instant beverage powder product” is meant a powder which can be converted to a liquid beverage by addition of a liquid, such as water.

In the context of the present invention the terms “beverage preparation” and “preparation” used as a substantive relate to any water-based liquid which can be ingested as a drink, e.g. by pouring, sipping or tube-feeding.

In the context of the present invention the term “protein fraction” relates to proteins of the composition in question e.g. the proteins of a powder or a beverage preparation.

In the context of the present invention the term “astringency” relates to a mouthfeeling. Astringency feels like a contraction of cheek muscles and results in increased saliva production. Thus, astringency is not a taste as such, but a physical mouth feeling and time-dependent feeling in the mouth.

In the context of the present invention the term “drying mouthfeeling” relates to a feeling in the mouth, it feels like a drying of the mouth and teeth and results in minimization of the saliva production.

Thus drying mouthfeeling is not a taste as such, but a physical mouth feeling and time-dependent feeling in the mouth.

In the context of the present invention the term “minerals” as used herein, unless otherwise specified, refers to any one of major minerals, trace or minor minerals, other minerals, and combinations thereof. Major minerals include calcium, phosphorus, potassium, sulfur, sodium, chlorine, magnesium. Trace or minor minerals include iron, cobalt, copper, zinc, molybdenum, iodine, selenium, manganese and other minerals include chromium, fluorine, boron, lithium, and strontium.

In the context of the present invention the terms “lipid”, “fat”, and “oil” as used herein unless otherwise specified, are used interchangeably to refer to lipid materials derived or processed from plants or animals. These terms also include synthetic lipid materials so long as such synthetic materials are suitable for human consumption.

In the context of the present invention the term “transparent” encompasses a beverage preparation having a visibly clear appearance and which allows light to pass and through which distinct images appear. A transparent beverage has a turbidity of at most 200 NTU.

In the context of the present invention the terms “opaque” encompasses a beverage preparation having a visibly unclear appearance and it has a turbidity of more than 200 NTU.

An aspect of the invention pertains to a packaged, heat-treated beverage preparation having a pH in the range of 2.0-4.7, the beverage comprising
a total amount of protein of 2 to 45% w/w relative to the weight of the beverage, wherein at least 85% w/w of the protein is BLG, and
optionally, sweetener, sugar polymers and/or flavour.

That the packaged, heat-treated beverage preparation comprises at least 85% w/w of the protein is very beneficial for a number of reasons. The high BLG content in acidic beverages also allow for increasing the pH range and decreasing the heating temperature while still maintaining clarity and lack of colour, this is possible even when a high protein concentration is applied. It was surprisingly found that the BLG beverages have a lower astringency, drying mouthfeeling, sourness, whey aroma and citric acid flavour compared to WPI beverages comprising a lower amount of BLG.

Another advantage of the present invention and the expanded pH range is that milky beverages can be produced having a high turbidity, low viscosity, while still being white and not becoming yellowish and still being stable.

In some preferred embodiments of the packaged, heat-treated beverage preparation of the invention at least 85% w/w of the protein is BLG. Preferably, at least 88% w/w of the protein is BLG, more preferably at least 90% w/w, even more preferably at least 91% w/w, and most preferably at least 92% w/w of the protein is BLG.

Even higher relative amounts of BLG are both feasible and desirable thus in some preferred embodiments of the invention at least 94% w/w of the protein of the packaged, heat-treated beverage preparation is BLG, more preferably at least 96% w/w of the protein is BLG, even more preferably at least 98% w/w of the protein is BLG, and most preferably approx. 100% w/w.

In some preferred embodiments of the invention the packaged, heat-treated beverage preparation is at least pasteurised.

In some preferred embodiments of the invention the packaged, heat-treated beverage preparation is sterilised.

In some preferred embodiments of the invention the native conformation of the proteins is maintained.

The degree of protein nativeness depends on a number of factors including protein concentration, pH, temperature and time of heat-treatment.

The intrinsic tryptophan fluorescence emission ratio R=I330/I350 is a measure of protein nativity. When R is at least 1.11 the native conformation is predominant, while when R is less than 1.11 an at least partial unfolding and aggregration is predominant. A method for analyzing the intrinsic tryptophan fluorescence is described in example 1.1.

The inventors have found that an intrinsic tryptophan fluorescence emission ratio R=I330/I350 of at least 1.11 can be obtained for heat-treated, high protein beverages, while still having a low viscosity, and being transparent. This is possible even when the protein fraction and/or beverage preparation is subjected to a heat-treatment corresponding to pasteurization (e.g. to a temperature below 90° C.).

Therefore, in some preferred embodiments of the invention the protein fraction of the beverage preparation has an intrinsic tryptophan fluorescence emission ratio (I330 nm/I350 nm) of at least 1.11, thus indicating that the proteins are in a native state.

In some preferred embodiments of the invention the protein fraction of the beverage preparation has an intrinsic tryptophan fluorescence emission ratio (I330 nm/I350 nm) of at least 1.12, preferably at least 1.13, more preferably at least 1.15, even more preferably at least 1.17, and most preferably at least 1.19.

In some preferred embodiments of the invention the packaged heat-treated beverage preparation comprising the protein fraction and optionally other ingredients, such as lipids, carbohydrates, vitamins, minerals, food acids or emulsifiers, have a tryptophan fluorescence emission ratio of at least 1.11.

Therefore, in some preferred embodiments of the invention the beverage preparation has an intrinsic tryptophan fluorescence emission ratio (I330 nm/I350 nm) of at least 1.11.

In some preferred embodiments of the invention the heat-treated beverage preparation has an intrinsic tryptophan fluorescence emission ratio (I330 nm/I350 nm) of at least 1.12, preferably at least 1.13, more preferably at least 1.15, even more preferably at least 1.17, and most preferably at least 1.19.

In some preferred embodiments of the invention the proteins are denatured or at least partly denatured.

Therefore, in some preferred embodiments of the invention the protein fraction of the beverage preparation has an intrinsic tryptophan fluorescence emission ratio (I330 nm/I350 nm) of less than 1.11, thus indicating that the proteins are at least partially unfolded and that aggregration is predominant.

In some embodiments of the invention the heat-treated beverage preparation has an intrinsic tryptophan fluorescence emission ratio (I330 nm/I350 nm) of less than 1.10, more preferably less than 1.08, even more preferably less than 1.05 and most preferably less than 1.00.

The beverage preparation may in addition to the protein fraction optionally also comprise other food additives, such as lipids, carbohydrates, vitamins, minerals, food acids or emulsifiers etc. In some preferred embodiments of the invention the beverage preparation has an intrinsic tryptophan fluorescence emission ratio (I330 nm/I350 nm) of less than 1.11, thus indicating that the proteins are at least partially unfolded and that aggregration is predominant.

In some preferred embodiments of the invention the heat-treated beverage preparation has an intrinsic tryptophan fluorescence emission ratio (I330 nm/I350 nm) of less than 1.10, more preferably less than 1.08, even more preferably less than 1.05 and most preferably less than 1.00.

Protein denaturation may also be described by another analysis method than by Tryptophan fluorescence. This method is described in example 1.3.

In some preferred embodiments of the invention, the protein fraction of the packaged, heat-treated beverage preparation has a degree of protein denaturation of at most 10%. Preferably at most 8%, more preferably at most 5%, even more preferably at most 3%, even more preferably at most 1%, and most preferably at most 0.5%.

In some preferred embodiments of the invention, the packaged, heat-treated beverage preparation has a degree of protein denaturation of at most 10%. Preferably at most 8%, more preferably at most 5%, even more preferably at most 3%, even more preferably at most 1%, and most preferably at most 0.5%.

In some embodiments of the invention, when the protein fraction and or the beverage preparation have been subjected for example to a high temperature heat-treatment, then the degree of protein denaturation is more than 10%, preferably more than 20%, preferably more than 30%, preferably more than 40%, or preferably more than 50%, or preferably more than 70%, or preferably more than 80%, or preferably more than 90%, or preferably more than 95%, or preferably more than 99%

In some preferred embodiments of the invention the packaged, heat-treated beverage preparation has a pH in the range of 3.0-4.3. These pH-ranges are particularly preferred for production of transparent beverages having low viscosity and improved taste.

Regarding the appearance it was surprisingly found that use of whey protein beverages wherein at least 85% w/w of the protein is BLG enables the possibility to increase the pH during thermal treatment, which provides improvements in visual perception (colour and turbidity) and in viscosity when compared to heat-treated WPI beverages.

It has surprisingly been found that there is a significant difference in the sensory parameters between beverages produced with WPI compared to the BLG beverages of the present invention. It was found that, surprisingly and advantageously, the BLG beverage had a lower level of astringency, drying mouth-feeling, sourness, whey aroma and citric acid flavour compared to a WPI beverage. It was furthermore found that by increasing the pH of an acidic beverage less sweetener was required to balance out the acidity of the beverage and a lower concentration of sweetener is therefore required in such beverages.

In some preferred embodiments of the invention the packaged heat-treated beverage preparation has a pH in the range of 3.0-4.1, or preferably 3.1-4.0 or preferably 3.2-3.9, or preferably 3.7-3.9, more preferably 3.4-3.9, and even more preferably 3.5-3.9.

These pH ranges are particularly relevant when the beverage preparation is pasteurised.

In some preferred embodiments of the invention the packaged heat-treated beverage preparation preferably has a pH in the range of 3.0-3.9, or preferably 3.2-3.7, or preferably 3.4-3.6. or preferably 3.5-3.7, or preferably 3.4-3.6.

These pH-ranges combined with high temperature treatment, such as sterilisation, are particularly relevant for production of transparent beverages having low viscosity and improved taste.

In some preferred embodiments of the invention the packaged, heat-treated beverage preparation has a pH in the range of 4.1-4.7, this pH range is particularly relevant for the production of stable beverages having a milky appearance and a high turbidity while still having a low viscosity. In some embodiments of the invention the pH range is of 4.2-4.6. In some other embodiments of the invention the pH range is of 4.2-4.5.

The visual appearance of the beverage preparation is of importance to the consumer both with respect to transparent and opaque beverages. Particularly for clear, water-like beverages, or white, milky beverages the inventors have found it advantageous to be able to control the colour of the beverage—or rather to control the lack of colour of the beverage.

However, even if dedicated colouring agents are added during the production of the beverage the inventors have found it advantageous to be able to avoid additional sources of colour to avoid unwanted variation or changes in the visual appearance of the beverage. The present inventors have found that the high BLG protein profile described herein is more colour neutral/colourless than conventional WPI and contributes with less colour variation that conventional WPI. Conventional WPI has a yellowish appearance which may be diminished to some extent by addition of an oxidizing agent such as bleach. However, addition of oxidizing agents is often not desirable and with the present invention it is not even necessary anymore.

The CIELAB colour scale as described in example 1.9 is used to determine the colour of a beverage. As an example a positive delta b*value indicates a colour that is more yellow than demineralized water whereas a negative delta b*value indicates a beverage that is more blue than demineralised water. It is therefore often preferred by the costumer that the colour delta b*value should be close to 0, in order to have a beverage that is neither yellow nor blue.

In some preferred embodiments of the present invention the packaged, heat-treated beverage preparation has a colour value delta b* in the range of −0.10 to +0.51 at the CIELAB colour scale, particularly if the preparation has a turbidity of at most 200 NTU, and more preferably at most 40 NTU.

In other preferred embodiments of the invention, the packaged, heat-treated beverage preparation has a colour value delta b* in the range of 0.0 to 0.40 at the CIELAB colour scale, preferably in the range of 0.10 to 0.25.

For opaque beverage preparations, e.g. having a turbidity above 200 NTU and preferably above 1000 NTU, the packaged, heat-treated beverage preparation preferably has a colour value delta b* at the CIELAB colour scale, in the range of −6 to −1.7; preferably in the range of −5.0 to −2.0.

In some preferred embodiments of the invention the protein fraction of the packaged heat-treated beverage preparation has a colour value delta b* in the range of −0.10 to +0.51, particularly if the preparation has a turbidity of at most 200 NTU, and more preferably at most 40 NTU.

These beverages have a less yellow colour compared to a beverage comprising WPI which had a higher delta b* value and a more yellow colour.

In other preferred embodiments of the invention, the protein fraction of the packaged heat-treated beverage preparation has a colour value delta b* in the range of 0.0 to 0.40 at the CIELAB colour scale, preferably in the range of 0.10 to 0.25.

The a*-value represents the green-red component, with green in the negative direction and red in the positive direction. It is often preferred that the colour delta a*value should be around zero, in order to have a beverage that is not red nor green.

It is typically preferred that the protein fraction of the packaged heat-treated beverage preparation has a delta a* is in the range of −0.2 to 0.2 at the CIELAB colour scale, particularly if the preparation has a turbidity of at most 200 NTU, and more preferably at most 40 NTU. Preferably, the packaged, heat-treated beverage preparation has a colour value delta a* in the range of −0.15 to 0.15 at the CIELAB colour scale, preferably in the range of −0.10 to 0.10.

The present inventors have found that it can be advantageous to control the mineral content to reach some of the desired properties of the packaged heat-treated beverage preparation.

In some embodiments of the invention the packaged heat-treated beverage preparation comprises a plurality of minerals. In one exemplary embodiment, the packaged heat-treated beverage preparation comprises at least four minerals. In one embodiment the four minerals are sodium, potassium, magnesium and calcium.

The present inventors have surprisingly found that when a BLG isolate is used as defined herein and in example 2, heat-treated beverage preparations having a high mineral concentration can be produced, without compromising the viscosity. This provides the possibility that packaged heat-treated beverage preparations can be produced having a high mineral content and that beverages that are nutritionally complete nutritional supplements or nutritionally incomplete supplements can be produced.

In some preferred embodiments of the invention the sum of the amounts of Na, K, Mg and Ca is within the range of 0 to 750 mM in the packaged, heat-treated beverage preparation, preferably within the range of 100-600 mM or preferably within the range of 200-500 mM.

In some preferred embodiments of the invention the sum of the amounts of Na, K, Mg and Ca is at most 750 mM in the packaged, heat-treated beverage preparation.

In other preferred embodiments of the invention the sum of the amounts of Na, K, Mg and Ca is at most 600 mM in the packaged, heat-treated beverage preparation, preferably at most 500 mM, or preferably at most 400 mM, or preferably at most 300 mM, or preferably at most 200 mM, preferably at most 170 mM, most preferably at most 150 mM, or preferably at most 130 mM, or preferably at most 100 mM or preferably at most 80 mM or preferably at most 60 mM or preferably at most 40 mM or preferably at most 30 mM or preferably at most 20 mM or preferably at most 10 mM or preferably at most 5 mM or preferably at most 1 mM.

In another exemplary embodiment, the packaged heat-treated beverage preparation comprises a plurality of minerals selected from the group consisting of: Calcium, Iodine, Zinc, Copper, Chromium, Iron, Phosphorus, Magnesium, Selenium, Manganese, Molybdenum, Sodium, Potassium, and combinations thereof.

In some preferred embodiments of the present invention the packaged heat-treated beverage preparation comprises at most 150 mM KCl and at most 150 mM CaCl₂), or the packaged heat-treated beverage preparation comprises at most 130 mM KCl and at most 130 mM CaCl₂) or the packaged heat-treated beverage preparation comprises at most 110 mM KCl and at most 110 mM CaCl₂) or the packaged heat-treated beverage preparation comprises at most 100 mM KCl and at most 100 mM CaCl₂) or preferably the packaged heat-treated beverage preparation comprises at most 80 mM KCl and at most 80 mM CaCl₂) or preferably the packaged heat-treated beverage preparation comprises at most 50 mM KCl and at most 50 mM CaCl₂) or preferably the packaged heat-treated beverage preparation comprises at most 40 mM KCl and at most 40 mM CaCl₂).

In other preferred embodiments of the invention the heat-treated beverage preparation is a low mineral beverage.

In the context of the present invention the term “low mineral” pertains to a composition, e.g. a liquid, beverage, a powder or another food product, that has at least one, preferably two, and even more preferably all, of the following:
an ash content of at most 1.2% w/w relative to total solids,
a total content of calcium and magnesium of at most 0.3% w/w relative to total solids,
a total content of sodium and potassium of at most 0.10% w/w relative to total solids,
a total content of phosphorus of at most 100 mg phosphorus per 100 g protein.

Preferably, a low mineral composition has at least one, preferably two or more, and even more preferably all, of the following:
an ash content of at most 0.7% w/w relative to total solids,
a total content of calcium and magnesium of at most 0.2% w/w relative to total solids,
a total content of sodium and potassium of at most 0.08% w/w relative to total solids, a total content of phosphorus of at most 80 mg phosphorus per 100 g protein.

Even more preferably, a low mineral composition has at least one, preferably two or more, and even more preferably all, of the following:
an ash content of at most 0.5% w/w relative to total solids,
a total content of calcium and magnesium of at most 0.15% w/w relative to total solids,
a total content of sodium and potassium of at most 0.06% w/w relative to total solids,
a total content of phosphorus of at most 50 mg phosphorus per 100 g protein.

It is particularly preferred that a low mineral composition has the following:
an ash content of at most 0.5% w/w relative to total solids,
a total content of calcium and magnesium of at most 0.15% w/w relative to total solids,
a total content of sodium and potassium of at most 0.06% w/w relative to total solids,
a total content of phosphorus of at most 50 mg phosphorus per 100 g protein.

The present inventors have found that the present invention makes it possible to prepare a packaged heat-treated beverage preparation having a very low content of phosphorus and other minerals such as Potassium, which is advantageous for patients suffering from kidney diseases or otherwise having a reduced kidney function.

The packaged heat-treated beverage preparation is preferably a low phosphorus beverage preparation.

The packaged heat-treated beverage preparation is preferably a low Potassium beverage preparation.

The packaged heat-treated beverage preparation is preferably low phosphorus and a low Potassium beverage preparation

In the context of the present invention the term “low phosphorus” pertains to a composition, e.g. a liquid, a powder or another food product, that has a total content of phosphorus of at most 100 mg phosphorus per 100 g protein. Preferably, a low phosphorus composition has a total content of at most 80 mg phosphorus per 100 g protein. More preferably, a low phosphorus composition may have a total content of at most 50 mg phosphorus per 100 g protein. Even more preferably, a low phosphorus composition may have a total content of phosphorus of at most 20 mg phosphorus per 100 g protein. Even more preferably, a low phosphorus composition may have a total content of phosphorus of at most 5 mg phosphorus per 100 g protein. Low phosphorus compositions according to the present invention may be used as a food ingredient for the production of a food product for patient groups that have a reduced kidney function.

Thus, in some particularly preferred embodiments of the invention the packaged heat-treated beverage preparation comprises at most 80 mg phosphorus per 100 g protein. Preferably, the packaged heat-treated beverage preparation comprises at most 30 mg phosphorus per 100 g protein. More preferably, the packaged heat-treated beverage preparation comprises at most 20 mg phosphorus per 100 g protein. Even more preferably, the packaged heat-treated beverage preparation comprises at most 10 mg phosphorus per 100 g protein. Most preferably, the packaged heat-treated beverage preparation comprises at most 5 mg phosphorus per 100 g protein.

The content of phosphorus relates to the total amount of elemental phosphorus of the composition in question and is determined according to Example 1.19.

In the context of the present invention the term “low potassium” pertains to a composition, e.g. a liquid, a powder or another food product, that has a total content of potassium of at most 700 mg potassium per 100 g protein. Preferably, a low phosphorus composition has a total content of at most 600 mg potassium per 100 g protein. More preferably, a low potassium composition may have a total content of at most 500 mg potassium per 100 g protein. More preferably, a low potassium composition may have a total content of potassium of at most 400 mg potassium per 100 g protein. More preferably, a low potassium composition may have a total content of potassium of at most 300 mg potassium per 100 g protein. Even more preferably, a low potassium composition may have a total content of potassium of at most 200 mg potassium per 100 g protein. Even more preferably, a low potassium composition may have a total content of potassium of at most 100 mg potassium per 100 g protein. Even more preferably, a low potassium composition may have a total content of potassium of at most 50 mg potassium per 100 g protein and even more preferably, a low potassium composition may have a total content of potassium of at most 10 mg potassium per 100 g protein.

Low potassium compositions according to the present invention may be used as a food ingredient for the production of a food product for patient groups that have a reduced kidney function.

Thus, in some particularly preferred embodiments of the invention the packaged heat-treated beverage preparation comprises at most 600 mg potassium per 100 g protein. More preferably, the packaged heat-treated beverage preparation comprises at most 500 mg potassium per 100 g protein. More preferably, the packaged heat-treated beverage preparation comprises at most 400 mg potassium per 100 g protein. More preferably, the packaged heat-treated beverage preparation comprises at most 300 mg potassium per 100 g protein. Even more preferably, the packaged heat-treated beverage preparation comprises at most 200 mg potassium per 100 g protein. Even more preferably, the packaged heat-treated beverage preparation comprises at most 100 mg potassium per 100 g protein. Even more preferably, the packaged heat-treated beverage preparation comprises at most 50 mg potassium per 100 g protein and even more preferably, the packaged heat-treated beverage preparation comprises at most 10 mg potassium per 100 g protein

The content of potassium relates to the total amount of elemental phosphorus of the composition in question and is determined according to Example 1.19.

In some preferred embodiments of the invention the packaged, heat-treated beverage preparation comprises at most 100 mg phosphorus/100 g protein and at most 700 mg potassium/100 g protein, preferably at most 80 mg phosphorus/100 g protein and at most 600 mg potassium/100 g protein, more preferably at most 60 mg phosphorus/100 g protein and at most 500 mg potassium/100 g protein, more preferably at most 50 mg phosphorus/100 g protein and at most 400 mg potassium/100 g protein, or more preferably at most 20 mg phosphorus/100 g protein and at most 200 mg potassium/100 g protein, or even more preferably at most 10 mg phosphorus/100 g protein and at most 50 mg potassium/100 g protein. In some preferred embodiments of the invention the packaged, heat-treated beverage preparation comprises at most 100 mg phosphor/100 g protein and at most 340 mg potassium/100 g protein.

The heat-treated beverage preparation comprising low amounts of phosphorus and Potassium may advantageously be supplemented with carbohydrates and lipids, the heat-treated beverage preparation preferably furthermore comprises a total amount of carbohydrates in a range between 30-60% of the total energy content of the beverage, preferably in a range between 35-50E % and a total amount of lipid in the range of 20-60% of the total energy content, preferably in a range between 30-50E %.

In one embodiment of the invention the packaged heat-treated beverage preparation comprises a plurality of vitamins. In one exemplary embodiment, the packaged heat-treated beverage preparation comprises at least ten vitamins. In one exemplary embodiment, the packaged heat-treated beverage preparation comprises a plurality of vitamins selected from the group consisting of: Vitamin A, vitamin B1, vitamin B2, vitamin B3, vitamin B5, vitamin B6, vitamin B7, vitamin B9, vitamin B12, vitamin C, vitamin D, vitamin K, Riboflavin, pantothenic Acid, vitamin E, thiamin, niacin, folic acid, biotin, and combinations thereof.

In one embodiment of the invention, the packaged heat-treated beverage comprises a plurality of vitamins and a plurality of minerals.

In some preferred embodiments of the present invention the packaged, heat-treated beverage preparation contains one or more food acids selected from the group consisting of citric acid, malic acid, tartaric acid, acetic acid, benzoic acid, butyric acid, lactic acid, fumaric acid, succinic acid, ascorbic acid, adipic acid, phosphoric acid, and mixtures thereof.

In an embodiment of the present invention, the packaged, heat-treated beverage preparation furthermore comprises a flavor selected from the group consisting of salt, flavorings, flavor enhancers and/or spices. In a preferred embodiment of the invention the flavor comprises chocolate, cocoa, lemon, orange, lime, strawberry, banana, forest fruit flavor or combinations thereof. The choice of flavor may depend on the beverage to be produced.

Transparency is a parameter that the consumer uses to evaluate the product. One way of determining the transparency of the liquid food product is by measuring the turbidity of the product as described in example 1.7.

In some embodiments of the packaged heat-treated beverage preparation it is beneficial that the beverage preparation is transparent. This may for example be advantageous when the beverage is used a sport beverage or in “protein water”, in which case it is beneficial that the beverage resembles water in appearance.

In a preferred embodiment of the present invention the packaged heat-treated beverage preparation has a turbidity of at most 200 NTU, such a beverage is transparent.

It has surprisingly been found by the inventors that transparent heat-treated beverage preparations has a turbidity of at most 200 NTU could be obtained by the heat-treated beverage preparation according to the invention.

This was found both when the heat-treatment applied was sterilization and pasteurisation.

In some preferred embodiments of the present invention the packaged, heat-treated beverage preparation has a turbidity of at most 150 NTU, or preferably a turbidity of at most 100 NTU, or preferably a turbidity of at most 80 NTU, or preferably a turbidity of at most 60 NTU or more preferably a turbidity of at most 40 NTU, or preferably a turbidity of at most 30 NTU, preferably a turbidity of at most 20 NTU, more preferably a turbidity of at most 10 NTU, and more preferably a turbidity of at most 5 NTU, even more preferably it has a turbidity of at most 2 NTU.

In a preferred embodiment of the present invention the packaged heat-treated beverage preparation has a turbidity of more than 200 NTU, such a beverage is opaque.

In some embodiments of the packaged heat-treated beverage preparation it is beneficial that the beverage preparation is opaque. This is for example advantageous when the beverage should resemble milk and have a milky appearance. The appearance of nutritionally complete nutritional supplements is typically opaque.

In some preferred embodiments of the invention the packaged, heat-treated beverage preparation has a turbidity of more than 250 NTU. Preferably the packaged, heat-treated beverage preparation has a turbidity of more than 300 NTU, more preferably it has a turbidity of more than 500 NTU, more preferably it has a turbidity of more than 1000, preferably a turbidity of more than 1500 NTU, even more preferably it has a turbidity of more than 2000 NTU.

The amount of insoluble matter in the heat-treated beverage preparation is a measure of the instability of the beverage and to which extent sedimentation of precipitated matter takes place over time. Beverages having a high amount of insoluble matter are typically considered unstable.

In the context of the present invention whey protein beverage preparations are considered “stable” if at most 15% of total protein in heated samples precipitated upon centrifugation at 3000×g for 5 minutes. See analysis method in example 1.10.

It has surprisingly been found that when BLG is used as the protein source in an amount of at least 85 w/w %, compared to when WPI having a lower BLG content is used as the protein source, then the protein fraction contains at most 15% insoluble matter after centrifugation at 3000 g for 5 minutes demonstrating that the beverage preparation is stable.

Therefore, in some preferred embodiments of the present invention, the protein fraction of the heat-treated beverage preparation contains at most 15% insoluble matter.

In some preferred embodiments of the present invention, the packaged, heat-treated beverage preparation contains at most 15% insoluble matter.

In some preferred embodiments of the present invention the packaged, heat-treated beverage preparation contains preferably at most 12% insoluble matter, more preferably at most 10% insoluble matter, even more preferably at most 8% insoluble matter, and most preferably at most 6% insoluble matter.

Even lower levels of insoluble matter are often preferred and in some preferred embodiments the packaged, heat-treated beverage preparation contains at most 4% insoluble matter, preferably at most 2% insoluble matter, more preferably at most 1% insoluble matter, and most preferably no detectable insoluble matter at all.

The consumer prefer that the heat-treated beverage is liquid, easy to drink and does not gel.

One way of determining the viscosity of the beverage preparation is by measuring the viscosity of the beverage as described in example 1.8.

In some embodiments of the packaged heat-treated beverage preparation it is beneficial that the beverage preparation is having a very low viscosity. This is advantageous when the beverage is used as a sport beverage or in some embodiments of a nutritionally complete nutritional supplement or a nutritionally incomplete supplement.

It has surprisingly been found by the inventors that beverage preparations having an acidic pH and that have been subjected to a heat-treatment such as pasteurisation and even to sterilisation had a viscosity of at most 200 centipoise (cP), measured at 22 degrees Celsius at a shear rate of 100/s.

Therefore, in some preferred embodiments of the present invention the packaged, heat-treated beverage preparation has a viscosity of at most 200 cP.

Preferably, the viscosity of the packaged, heat-treated beverage preparation is at most 150 cP, preferably at most 100 cP, more preferably at most 80 cP, even more preferably at most 50 cP, and most preferably at most 40 cP.

Even lower viscosity is often preferred, thus in some preferred embodiments of the invention the viscosity of the packaged, heat-treated beverage preparation is at most 20 cP, preferably at most 10 cP, more preferably at most 5 cP, even more preferably at most 3 cP, even more preferably at most 2 cP, and most preferably at most 1 cP.

It has previously been found that in order to produce acidic transparent heat-treated beverages comprising WPI, wherein the beverage is having a pH above pH 3.0, it was essential to add an antiaggregant to the beverage, see for example Etzel 2004 (Etzel, M. R., 2004, Manufacture and use of dairy protein fraction. American Society for Nutritional Science, pp. 996-1002).

It was surprisingly found by the inventors that transparent heat-treated beverages comprising at least 85% w/w BLG can be produced even at a pH higher than pH 3.0 without the addition of an antiaggregant.

Therefore, in some preferred embodiments of the present invention the packaged, heat-treated beverage preparation does not comprise any antiaggregant or alternatively only traces of antiaggregant.

In the context of the present invention the term “antiaggregant” pertains to food grade, non-protein surfactants such as e.g. lauryl sulfate, polysorbate, and mono- and/or di-glycerides.

In some embodiments of the invention the packaged, heat-treated beverage preparation comprises at most 0.1% w/w antiaggregant, preferably at most 0.03% w/w antiaggregant, and most preferably no antiaggregant. The embodiments are particularly preferred in relation to transparent, low fat beverages.

In some preferred embodiments of the present invention the packaged, heat-treated beverage preparation comprises a total amount of protein of 4.0 to 30% w/w relative to the weight of the beverage.

In some embodiments of the invention it is advantageous that the packaged heat-treated beverage preparation has a protein content of 2.0 to 10.0% w/w relative to the weight of the beverage.

Therefore, in some embodiments of the invention the packaged heat-treated beverage preparation preferably comprises a total amount of protein of 2.0 to 10% w/w relative to the weight of the beverage, preferably a total amount of protein of 3.0 to 10% w/w relative to the weight of the beverage, preferably a total amount of protein of 5.0 to 9.0% w/w relative to the weight of the beverage, preferably a total amount of protein of 6.0 to 8.0% w/w relative to the weight of the beverage.

In some embodiments of the invention it is advantageous that the protein content of the beverage is high such as 10.0 to 45.0% w/w relative to the weight of the beverage.

Therefore in some embodiments of the present invention the packaged heat-treated beverage preparation preferably comprises a total amount of protein of 10.0 to 45.0% w/w relative to the weight of the beverage, preferably a total amount of protein of 10.0 to 20% w/w relative to the weight of the beverage, preferably a total amount of protein of 12 to 30% w/w relative to the weight of the beverage, preferably a total amount of protein of 15 to 25% w/w relative to the weight of the beverage, preferably a total amount of protein of 18 to 20% w/w relative to the weight of the beverage.

The packaged heat-treated beverage preparation of the invention is particularly useful as a sport beverage in which case it preferably contains optionally only a limited amount of lipid and/or optionally also a limited amount of carbohydrates.

In some preferred embodiments of the present invention the preparation is particularly useful as a sport beverage and comprises e.g. a total amount of protein in the range of 2-45% w/w relative to the weight of the beverage, preferably 2-20% w/w relative to the weight of the beverage, or preferably 2-10% w/w relative to the weight of the beverage, most preferably 2-6% w/w relative to the weight of the beverage.

In some preferred embodiments of the present invention the packaged heat-treated beverage preparation is particularly useful as a nutritionally incomplete nutritional supplement and comprises e.g. a total amount of protein in the range of 2-45% w/w relative to the weight of the beverage, preferably 2-20% w/w relative to the weight of the beverage, or preferably 3-10% w/w relative to the weight of the beverage.

In some preferred embodiments of the present invention the packaged heat-treated beverage preparation is particularly useful as a nutritionally complete nutritional supplement and comprises e.g. a total amount of protein in the range of 4-45% w/w relative to the weight of the beverage or preferably 5-20% w/w relative to the weight of the beverage.

In some preferred embodiments of the present invention the packaged heat-treated beverage preparation is particularly advantageous for patients suffering from kidney diseases or otherwise having a reduced kidney function.

In some preferred embodiments of the present invention the packaged heat-treated beverage preparation comprises e.g. a total amount of protein in the range of 2-45% w/w relative to the weight of the beverage, preferably 2-20% w/w relative to the weight of the beverage, or preferably 3-12% w/w relative to the weight of the beverage, or preferably 3-10% w/w relative to the weight of the beverage.

It is particularly preferred that the packaged heat-treated beverage preparation comprises a BLG isolate, e.g. in combination with other protein sources, preferably as the main protein source and possibly even as the only protein source.

The packaged, heat-treated beverage preparation of the present invention may comprise other macronutrients than proteins. In some embodiments of the invention the packaged, heat-treated beverage preparation furthermore comprises carbohydrates. The total carbohydrate content in the heat-treated beverage preparation of the invention depends on the intended use of the heat-treated beverage preparation.

In some preferred embodiments of the invention, the packaged heat-treated beverage preparation furthermore comprises at least one source of carbohydrate. In one exemplary embodiment, the at least one source of carbohydrate is selected from the group consisting of: sucrose, maltodextrin, corn syrup solids, saccharose, maltose, sucromalt, maltitol powder, glycerine, glucose polymers, corn syrup, modified starches, resistant starches, rice-derived carbohydrates, isomaltulose, white sugar, glucose, fructose, lactose, high fructose corn syrup, honey, sugar alcohols, fructooligosaccharides, soy fiber, corn fiber, guar gum, konjac flour, polydextrose, Fibersol, and combinations thereof.

In some preferred embodiments the packaged, heat-treated beverage preparation furthermore comprises carbohydrates in a range between 0 to 95% of the total energy content of the preparation, preferably in a range between 10 to 85% of the total energy content of the preparation, preferably in a range between 20 to 75% of the total energy content of the preparation or preferably in a range between 30 to 60% of the total energy content of the preparation.

Even lower carbohydrate content is often preferred, thus in some preferred embodiments of the invention preferably in a range between 0 to 30% of the total energy content of the preparation more preferably in a range between 0 to 20% of the total energy content of the preparation even more preferably in a range between 0 to 10% of the total energy content of the preparation.

In some preferred embodiments of the present invention the preparation is particularly useful as a sport beverage and comprises a total amount of carbohydrate of at most 75% of the total energy content of the beverage (E), preferably at most 40E %, preferably at most 10 E % or preferably at most 5 E %.

In some preferred embodiments of the present invention the packaged heat-treated beverage preparation is particularly useful as a nutritionally incomplete nutritional supplement and comprises a total amount of carbohydrate in a range between 70-95% of the total energy content of the beverage (E), preferably 80-90E %.

In some preferred embodiments of the present invention the packaged heat-treated beverage preparation is particularly useful as a nutritionally complete nutritional supplement and comprises a total amount of carbohydrate in a range between 30-60% of the total energy content of the beverage, preferably in a range between 35-50E %.

In some preferred embodiments of the present invention the packaged heat-treated beverage preparation is particularly advantageous for patients suffering from kidney diseases or otherwise having a reduced kidney function.

In some preferred embodiments of the present invention the packaged heat-treated beverage preparation comprises a total amount of carbohydrate in a range between 30-60% of the total energy content of the beverage, preferably in a range between 35-50E %.

In one embodiment of the invention the packaged, heat-treated beverage preparation furthermore comprises at least one additional ingredient selected from the group consisting of vitamins, flavouring agent, minerals, sweeteners, antioxidants, food acid, lipids, carbohydrate, prebiotics, probiotics and non-whey protein.

In one embodiment of the invention, the liquid solution furthermore comprises at least one high intensity sweetener. In one embodiment, the at least one high intensity sweetener is selected from the group consisting of aspartame, cyclamate, sucralose, acesulfame salt, neotame, saccharin, Stevia extract, a steviol glycoside such as e.g. rebaudioside A, or a combination thereof. In some embodiments of the invention, it is particularly preferred that the sweetener comprises or even consists of one or more high intensity sweeteners (HIS).

HIS are both found among both natural and artificial sweeteners and typically have a sweetening intensity of at least 10 times that of sucrose.

If used, the total amount of HIS is typically in the range of 0.01-2% w/w. For example, the total amount of HIS may be in the range of 0.05-1.5% w/w. Alternatively, the total amount of HIS may be in the range of 0.1-1.0% w/w.

The choice of the sweetener may depend on the beverage to be produced, e.g. high-intensity sugar sweeteners (e.g. aspartame, acetsulfam-K or sucralose) may be used in beverage where no energy contribution from the sweetener is desired, whereas for beverages having a natural profile natural sweeteners (e.g. steviol glycosides, sorbitol or sucrose) may be used.

It may furthermore be preferred that the sweetener comprises or even consists of one or more polyol sweetener(s). Non-limiting examples of useful polyol sweetener are maltitol, mannitol, lactitol, sorbitol, inositol, xylitol, threitol, galactitol or combinations thereof. If used, the total amount of polyol sweetener is typically in the range of 1-20% w/w. For example, the total amount of polyol sweetener may be in the range of 2-15% w/w. Alternatively, the total amount of polyol sweetener may be in the range of 4-10% w/w.

The packaged, heat-treated beverage preparation of the present invention may comprise other macronutrients than proteins. In some embodiments of the invention the packaged, heat-treated beverage preparation furthermore comprises lipids. The total lipid content in the heat-treated beverage preparation of the invention depends on the intended use of the heat-treated beverage preparation.

In some preferred embodiments of the invention, the packaged, heat-treated beverage preparation has a lipid content between 0 to 50% of the total energy content of the preparation, or preferably in a range between 0 to 45% of the total energy content of the preparation, or preferably in a range between 0 to 30% of the total energy content of the preparation or preferably in a range between 0 to 20% of the total energy content of the preparation or preferably in a range between 0 to 10% of the total energy content of the preparation or preferably in a range between 0 to 5% of the total energy content of the preparation.

The amount of lipid is determined according to ISO 1211:2010 (Determination of Fat Content—Röse-Gottlieb Gravimetric Method).

In some preferred embodiments of the present invention the preparation is particularly useful as a sport beverage and comprises e.g. a total amount of lipid of at most 10 E %, preferably at most at most 1E %.

In some preferred embodiments of the present invention the packaged heat-treated beverage preparation is particularly useful as a nutritionally incomplete nutritional supplement and comprises e.g. a total amount of lipid of at most 10% of the total energy content of the beverage, preferably at most 1E %.

In some preferred embodiments of the present invention the packaged heat-treated beverage preparation is particularly useful as a nutritionally complete nutritional supplement and comprises e.g. a total amount of lipid in the range of 20-50% of the total energy content, preferably in a range between 30-40E %.

In some preferred embodiments of the present invention the packaged heat-treated beverage preparation is particularly advantageous for patients suffering from kidney diseases or otherwise having a reduced kidney function.

In some preferred embodiments of the present invention the packaged heat-treated beverage preparation comprises e.g. a total amount of lipid in the range of 20-60% of the total energy content, preferably in a range between 30-50E %.

In some preferred embodiments of the invention, the sum of alpha-lactalbumin (ALA) and caseinomacropeptide (CMP) comprises at least 40% w/w of the non-BLG protein of the powder, preferably at least 60% w/w, even more preferably at least 70% w/w, and most preferably at least 90% w/w of the non-BLG protein of the powder.

In other preferred embodiments of the invention, each main non-BLG whey protein is present in a weight percentage relative to total protein which is at most 25% of its weight percentage relative to total protein in a standard whey protein concentrate from sweet whey, preferably at most 20%, more preferably at most 15%, even more preferably at most 10%, most preferably at most 6%.

Even lower concentrations of the main non-BLG whey proteins may be desirable. Thus, in additional preferred embodiments of the invention, each main non-BLG whey protein is present in a weight percentage relative to total protein which is at most 4% of its weight percentage relative to total protein in a standard whey protein concentrate from sweet whey, preferably at most 3%, more preferably at most 2%, even more preferably at most 1%.

The inventors have seen indications that reduction of lactoferrin and/or lactoperoxidase is particularly advantageous for obtaining a colour-neutral whey protein product.

Thus in some preferred embodiments of the invention, lactoferrin is present in a weight percentage relative to total protein which is at most 25% of its weight percentage relative to total protein in a standard whey protein concentrate from sweet whey, preferably at most 20%, more preferably at most 15%, even more preferably at most 10%, most preferably at most 6%. Even lower concentrations of lactoferrin may be desirable. Thus, in additional preferred embodiments of the invention, lactoferrin is present in a weight percentage relative to total protein which is at most 4% of its weight percentage relative to total protein in a standard whey protein concentrate from sweet whey, preferably at most 3%, more preferably at most 2%, even more preferably at most 1%.

Similarly, in some preferred embodiments of the invention, lactoperoxidase is present in a weight percentage relative to total protein which is at most 25% of its weight percentage relative to total protein in a standard whey protein concentrate from sweet whey, preferably at most 20%, more preferably at most 15%, even more preferably at most 10%, most preferably at most 6%. Even lower concentrations of lactoperoxidase may be desirable. Thus, in additional preferred embodiments of the invention, lactoperoxidase is present in a weight percentage relative to total protein which is at most 4% of its weight percentage relative to total protein in a standard whey protein concentrate from sweet whey, preferably at most 3%, more preferably at most 2%, even more preferably at most 1%.

Lactoferrin and lactoperoxidase are quantified according to Example 1.29 In an embodiment of the invention the packaged heat-treated beverage preparation is a sports beverage.

In an embodiment of the invention the packaged heat-treated beverage preparation is a nutritionally complete nutritional supplement.

In an embodiment of the invention the packaged heat-treated beverage preparation is a nutritionally incomplete nutritional supplement.

In an embodiment of the invention the packaged heat-treated beverage preparation is a low phosphorus and low potassium beverage suitable for patients suffering from kidney diseases or otherwise having a reduced kidney function.

The packaged heat-treated beverage preparation of the invention is particularly useful as a sport beverage in which case it preferably contains optionally only a limited amount of lipid and/or optionally also a limited amount of carbohydrates.

In some preferred embodiments of the present invention the preparation is particularly useful as a sport beverage and comprises e.g.:
a total amount of protein in the range of 2-45% w/w relative to the weight of the beverage, preferably 2-20% w/w relative to the weight of the beverage, or preferably 2-10% w/w relative to the weight of the beverage, most preferably 2-6% w/w relative to the weight of the beverage
a total amount of carbohydrate of at most 75% of the total energy content of the beverage (E), preferably at most 40E %, preferably at most 10 E % or preferably at most 5 E % and
a total amount of lipid of at most 10 E %, preferably at most at most 1E %.

In some preferred embodiments of the present invention the packaged heat-treated beverage preparation is particularly useful as a nutritionally incomplete nutritional supplement and comprises e.g.:
a total amount of protein in the range of 2-45% w/w relative to the weight of the beverage, preferably 2-20% w/w or preferably 3-10% w/w relative to the weight of the beverage
a total amount of carbohydrate in a range between 70-95% of the total energy content of the beverage (E), preferably 80-90E %, and
a total amount of lipid of at most 10% of the total energy content of the beverage, preferably at most 1E %.

In some preferred embodiments of the present invention the packaged heat-treated beverage preparation is particularly useful as a nutritionally complete nutritional supplement and comprises e.g.:
a total amount of protein in the range of 4-45% w/w relative to the weight of the beverage, preferably 5-20% w/w relative to the weight of the beverage
a total amount of carbohydrate in a range between 30-60% of the total energy content of the beverage, preferably in a range between 35-50E % and
a total amount of lipid in the range of 20-50% of the total energy content, preferably in a range between 30-40E %.

In some preferred embodiments of the present invention the packaged heat-treated beverage preparation is particularly advantageous for patients suffering from kidney diseases or otherwise having a reduced kidney function. The beverage preparation is having a very low content of phosphorus and other minerals such as Potassium.

In some preferred embodiments of the present invention the packaged heat-treated beverage preparation comprises e.g.:
a total amount of protein in the range of 2-45% w/w relative to the weight of the beverage, preferably 2-20% w/w relative to the weight of the beverage or preferably 3-12% w/w, preferably 3-10% w/w relative to the weight of the beverage,
a total amount of carbohydrate in a range between 30-60% of the total energy content of the beverage, preferably in a range between 35-50E % and
a total amount of lipid in the range of 20-60% of the total energy content, preferably in a range between 30-50E %.

An aspect of the invention pertains to a method of producing a packaged, heat-treated beverage preparation having a pH in the range of 2-4.7, comprising the following steps:

a) Providing a liquid solution comprising:
a total amount of protein of 2 to 45% by weight, wherein at least 85% of the protein is BLG
optionally, sweetener, sugar polymers and/or flavour

b) packaging the liquid solution,

wherein the liquid solution of step a) and/or the packaged liquid solution of step b) is subjected to a heat-treatment comprising at least pasteurisation.

In some preferred embodiments the liquid solution of the invention at least 85% w/w of the protein is BLG. Preferably, at least 88% w/w of the protein is BLG, more preferably at least 90% w/w, even more preferably at least 91% w/w, and most preferably at least 92% w/w of the protein is BLG.

Even higher relative amounts of BLG are both feasible and desirable thus in some preferred embodiments of the invention at least 94% w/w of the protein of the liquid solution is BLG, more preferably at least 96% w/w of the protein is BLG, even more preferably at least 98% w/w of the protein is BLG, and most preferably approx. 100% w/w.

The packaging of step b) may be any suitable packaging techniques, and any suitable container may be used for packaging the liquid solution.

However, in a preferred embodiment of the invention, the packaging of step b) is aseptic packaging, i.e. the liquid solution is packaged under aseptic conditions. For example, the aseptic packaging may be performed by using an aseptic filling system, and it preferably involves filling the liquid solution into one or more aseptic container(s).

Aseptic filling and sealing is particularly preferred if the liquid solution already is sterile or very low in microorganisms prior to filling.

Examples of useful containers are e.g. bottles, cartons, bricks, and/or bags.

In some preferred embodiment of the method the packaged liquid solution of step b) is subjected to a heat-treatment comprising at least pasteurisation. The embodiment is typically referred to as in-container heat-treatment or retort treatment and involves heating the entire container and its contents to achieve pasteurization or even sterility. When using in-container heat-treatment it is particularly preferred that the temperature is kept in the range 70-82 degrees C., more preferably in the range 70-80 degrees C., and most preferably in the range 70-78 degrees C. In this way the level of protein unfolding is kept to a minimum.

In other preferred embodiments of the inventive method the liquid solution of step a) is subjected to a heat-treatment comprising at least pasteurisation and then subsequently packaged in step b).

In particularly preferred embodiments heat-treatment involves heating the beverage preparation to a temperature in the range of 70-82 degrees C.

In some preferred embodiments of the invention the temperature of the heat-treatment is in the range 70-80 degrees C., preferably in the range 70-79 degrees C., more preferably in the range 71-78 degrees C., even more preferably in the range 72-77 degrees C., and most preferably in the range 73-76 degrees C., such as approx. 75 degrees C.

Preferably, the duration of the heat-treatment, when performed in the temperature range 70-82, is 1 second to 30 minutes. The highest exposure times are best suited for the lowest temperatures of the temperature range and vice versa. The lower the pH of the liquid solution the higher temperature can be tolerated without unfolding.

In particularly preferred embodiments of the invention the heat-treatment provides 70-80 degrees C. for 1 second to 30 minutes, more preferably 71-77 degrees C. for 1 minute to 25 minutes, and even more preferred 72-76 degrees C. for 2 minute to 20 minutes.

In some preferred embodiments of the invention the heat-treatment involves heating to a temperature of 85° C.-95 degrees C. for 1 to 3 minutes.

Higher temperatures may also be preferred in some embodiments, especially if unfolding and optionally also aggregation for BLG is required. For example, the temperature of the heat-treatment may be at least 81 degrees C., preferably at least 91 degrees C., preferably at least 95 degrees C., more preferred at least 100 degrees C., even more preferred at least 120 degrees C., and most preferred at least 140 degrees C.

In some preferred embodiments of the invention the sterilisation involves a temperature in the range of 120 to 150 degrees C. for 4 to 30 seconds.

The heat-treatment may for example involve a temperature in the range of 90-130 degrees C. and a duration in the range of 5 seconds-10 minutes. The heat-treatment may e.g. involve heating to a temperature in the range of 90-95 degrees C. for a duration of 1-10 minutes, e.g. approx. 120 degrees C. for 20 approx. seconds. Alternatively, the heat-treatment may involve heating to a temperature in the range of 115-125 degrees C. for a duration of 5-30 seconds, e.g. approx. 120 degrees C. for approx. 20 seconds.

Alternatively, the heat-treatment may for example be a UHT-type treatment which typically involves a temperature in the range of 135-144 degrees C. and a duration in the range of 2-10 seconds.

Alternatively, but also preferred, the heat-treatment may involve a temperature in the range of 145-180 degrees C. and a duration in the range of 0.01-2 seconds, and more preferably a temperature in the range of 150-180 degrees C. and a duration in the range of 0.01-0.3 seconds.

The implementation of the heat-treatment may involve the use of equipment such as a plate or tubular heat exchanger, scraped surface heat exchanger or a retort system. Alternatively, and particularly preferred for heat-treatments above 95 degrees C., direct steam-based heating may be employed, e.g. using direct steam injection, direct steam infusion, or spray-cooking. Additionally, such direct steam-based heating is preferably used in combination with flash cooling. Suitable examples of implementation of spray-cooking are found in WO2009113858A1, which are incorporated herein for all purposes. Suitable examples of implementation of direct steam injection and direct steam infusion are found in WO2009113858A1 and WO 2010/085957 A3, which are incorporated herein for all purposes. General aspects of high temperature treatment are e.g. found in “Thermal technologies in food processing” ISBN 185573558 X, which is incorporated herein by reference for all purposes.

In some preferred embodiments of the invention the pasteurisation is combined with another physical microbial reduction.

Useful examples of physical microbial reduction involve one or more of germ filtration, UV radiation, high pressure treatment, pulsed electric field treatment, and ultrasound.

In some particularly preferred embodiments of the invention the heat-treatment is selected so that it provides a degree of protein denaturation of at most 50%, preferably at most 20%, even more preferred at most 10%, and most preferred at most 5%.

It is furthermore preferred that the heat-treatment is selected so that is provides an intrinsic tryptophan fluorescence ratio (I330/I350) of at least 1.11, preferably at least 1.13, more preferably at least 1.15, and even more preferred at least 1.17.

In some preferred embodiments of the invention the heat-treatment is a sterilization resulting in a sterile liquid beverage preparation. Such a sterilisation may e.g. be obtained by combining germ filtration and heat-treatment, e.g. pasteurisation. The sterilisation may e.g. involve heat-treatment followed by germ filtration, or even more preferred germ filtration followed by heat-treatment.

Depending on the used heat-treatment temperatures it is beneficial that the beverage preparation is subjected to cooling. According to a preferred aspect of the inventive process, following the heat-treatment, the heat-treated beverage preparation is in an optional step cooled to preferably 0 to 50 degrees C., preferably 0 to 25 degrees C. or preferably 0 to 20 degrees C. or preferably 0 to 15 degrees C., preferably 0 to 10 degrees C. or preferably 4 to 8 degrees C. or preferably 2 to 5 degrees C. or preferably 1 to 5 degrees C.

If the beverage preparation has been pasteurized it is preferably cooled to 0 to 15 degrees C. after the heat-treatment, preferably to 1 to 10 degrees C., and more preferably to 1 to 6 degrees C.

According to an embodiment of the method, generally any acid or base may be used to adjust the pH, Those skilled in the art will recognize means suitable for adjusting the pH. Suitable acids include, e.g. citric acid, hydrochloric acid, malic acid or tartaric acid, or phosphoric acid most preferably citric acid and/or phosphoric acid.

Useful examples of useful bases are hydroxide salts, e.g. sodium hydroxide or potassium hydroxide, carbonate salts or hydrocarbonate salts, carboxylate salts such as e.g. citrate salts or lactic acid salts and combinations thereof. Preferably, a base such as KOH or NaOH is employed to adjust the pH.

In some preferred embodiments of the invention the liquid solution has a pH in the range of 3.0-4.3. These pH-ranges are particularly preferred for production of transparent beverages having low viscosity and improved taste.

Regarding the appearance it was surprisingly found that use of whey protein beverages wherein at least 85% w/w of the protein is BLG enables the possibility to increase the pH during thermal treatment, which provides improvements in visual perception (colour and turbidity) and in viscosity when compared to heat-treated WPI beverages.

It has surprisingly been found that there is a significant difference in the sensory parameters between beverages produced with WPI compared to the BLG beverages of the present invention. It was found that, surprisingly and advantageously, the BLG beverage had a lower level of astringency, drying mouth-feeling, sourness, whey aroma and citric acid flavour compared to a WPI beverage. It was furthermore found that by increasing the pH of an acidic beverage less sweetener was required to balance out the acidity of the beverage and a lower concentration of sweetener is therefore required in such beverages.

In some preferred embodiments of the invention the packaged heat-treated beverage preparation has a pH in the range of 3.0-4.1, or preferably 3.1-4.0 or preferably 3.2-3.9, or preferably 3.7-3.9, more preferably 3.4-3.9, and even more preferably 3.5-3.9.

These pH ranges are particularly relevant when the beverage preparation is pasteurised.

In some preferred embodiments of the invention the liquid solution preferably has a pH in the range of 3.0-3.9, or preferably 3.2-3.7, or preferably 3.4-3.6 or preferably 3.5-3.7, or preferably 3.4-3.6.

These pH-ranges combined with high temperature treatment, such as sterilisation, are particularly relevant for production of transparent beverages having low viscosity and improved taste.

In some preferred embodiments of the invention the liquid solution has a pH in the range of 4.1-4.7, this pH range is particularly relevant for the production of stable beverages having a milky appearance and a high turbidity while still having a low viscosity. In some embodiments of the invention the pH range is of 4.2-4.6. In some other embodiments of the invention the pH range is of 4.2-4.5.

In some preferred embodiments of the present invention the liquid solution comprises a total amount of protein of 4.0 to 30% w/w relative to the weight of the beverage.

In some embodiments of the invention it is advantageous that the liquid solution has a protein content of 2.0 to 10.0% w/w relative to the weight of the solution.

Therefore, in some embodiments of the invention the liquid solution, preferably comprises a total amount of protein of 2.0 to 10% w/w relative to the weight of the liquid solution, preferably a total amount of protein of 3.0 to 10% w/w relative to the weight of the liquid solution, preferably a total amount of protein of 5.0 to 9.0% w/w relative to the weight of the liquid solution, preferably a total amount of protein of 6.0 to 8.0% w/w relative to the weight of the liquid solution.

In some embodiments of the invention it is advantageous that the protein content of the liquid solution is high such as 10.0 to 45.0% w/w relative to the weight of the liquid solution.

Therefore in some embodiments of the present invention the liquid solution preferably comprises a total amount of protein of 10.0 to 45.0% w/w relative to the weight of the liquid solution, preferably a total amount of protein of 10.0 to 20% w/w relative to the weight of the liquid solution, preferably a total amount of protein of 12 to 30% w/w relative to the weight of the liquid solution, preferably a total amount of protein of 15 to 25% w/w relative to the weight of the liquid solution, preferably a total amount of protein of 18 to 20% w/w relative to the weight of the liquid solution.

It is particularly preferred that the liquid solution comprises a BLG isolate, e.g. in combination with other protein sources, preferably as the main protein source and possibly even as the only protein source.

The BLG isolate is preferably a BLG isolate powder or a liquid BLG isolate contain water and the solids of the BLG isolate powder in an amount in the range from 1-50% w/w.

The beta-lactoglobulin (BLG) isolate powder, preferably prepared by spray-drying, has a pH in the range of i) 2-4.9, ii) 6.1-8.5, or iii) 5.0-6.0 and comprises:
total protein in an amount of at least 30% w/w,
BLG in an amount of at least 85% w/w relative to total protein, and
water in an amount of at most 10% w/w.

The BLG isolate powder preferably has one or more of the following:
a bulk density of at least 0.2 g/cm³,
an intrinsic tryptophan fluorescence emission ratio (I330/I350) of at least 1.11,
a degree of protein denaturation of at most 10%,
a heat-stability at pH 3.9 of at most 200 NTU, and
at most 1000 colony-forming units/g.

The BLG isolate powder is preferably an edible composition.

In some preferred embodiments of the invention, the BLG isolate powder has a pH in the range of 2-4.9. Such powders are particularly useful for acidic food products and particularly acidic beverages.

In other preferred embodiments of the invention, BLG isolate powder has a pH in the range of 6.1-8.5.

In some preferred embodiments of the invention, the BLG isolate powder comprises total protein in an amount of at least 40% w/w, preferably at least 50% w/w, at least 60% w/w, more preferably at least 70% w/w, even more preferably at least 80% w/w.

Even higher protein contents may be required and in some preferred embodiments of the invention, the BLG isolate powder comprises total protein in an amount of at least 85% w/w, preferably at least 90% w/w, at least 92% w/w, more preferably at least 94% w/w, and even more preferably at least 95% w/w.

Total protein is measured according to Example 1.5.

In some preferred embodiments of the invention, the BLG isolate powder comprises BLG in an amount of at least 92% w/w relative to total protein, preferably at least 95% w/w, more preferably at least 97% w/w, even more preferably at least 98%, and most preferably BLG in an amount of at least 99.5% w/w relative to total protein.

In some preferred embodiments of the invention, the sum of alpha-lactalbumin (ALA) and caseinomacropeptide (CMP) comprises at least 40% w/w of the non-BLG protein of the powder, preferably at least 60% w/w, even more preferably at least 70% w/w, and most preferably at least 90% w/w of the non-BLG protein of the powder.

In other preferred embodiments of the invention, each main non-BLG whey protein is present in a weight percentage relative to total protein which is at most 25% of its weight percentage relative to total protein in a standard whey protein concentrate from sweet whey, preferably at most 20%, more preferably at most 15%, even more preferably at most 10%, most preferably at most 6%.

Even lower concentrations of the main non-BLG whey proteins may be desirable. Thus, in additional preferred embodiments of the invention, each main non-BLG whey protein is present in a weight percentage relative to total protein which is at most 4% of its weight percentage relative to total protein in a standard whey protein concentrate from sweet whey, preferably at most 3%, more preferably at most 2%, even more preferably at most 1%.

The inventors have seen indications that reduction of lactoferrin and/or lactoperoxidase is particularly advantageous for obtaining a colour-neutral whey protein product.

Thus in some preferred embodiments of the invention, lactoferrin is present in a weight percentage relative to total protein which is at most 25% of its weight percentage relative to total protein in a standard whey protein concentrate from sweet whey, preferably at most 20%, more preferably at most 15%, even more preferably at most 10%, most preferably at most 6%. Even lower concentrations of lactoferrin may be desirable. Thus, in additional preferred embodiments of the invention, lactoferrin is present in a weight percentage relative to total protein which is at most 4% of its weight percentage relative to total protein in a standard whey protein concentrate from sweet whey, preferably at most 3%, more preferably at most 2%, even more preferably at most 1%.

Similarly, in some preferred embodiments of the invention, lactoperoxidase is present in a weight percentage relative to total protein which is at most 25% of its weight percentage relative to total protein in a standard whey protein concentrate from sweet whey, preferably at most 20%, more preferably at most 15%, even more preferably at most 10%, most preferably at most 6%. Even lower concentrations of lactoperoxidase may be desirable. Thus, in additional preferred embodiments of the invention, lactoperoxidase is present in a weight percentage relative to total protein which is at most 4% of its weight percentage relative to total protein in a standard whey protein concentrate from sweet whey, preferably at most 3%, more preferably at most 2%, even more preferably at most 1%.

Lactoferrin and lactoperoxidase are quantified according to Example 1.29.

In some preferred embodiments of the invention, the BLG isolate powder has a water content in an amount of at most 10% w/w, preferably at most 7% w/w, more preferably at most 6% w/w, even more preferably at most 4% w/w, and most preferred at most 2% w/w.

In some preferred embodiments of the invention the BLG isolate powder comprises carbohydrate in an amount of at most 60% w/w, preferably at most 50% w/w, more preferably at most 20% w/w, even more preferably at most 10% w/w, even more preferably at most 1% w/w, and most preferably at most 0.1%. The BLG isolate powder may for example contain carbohydrates, such as e.g. lactose, oligosaccharides and/or hydrolysis products of lactose (i.e. glucose and galactose), sucrose, and/or maltodextrin.

In some preferred embodiments of the invention, the BLG isolate powder comprises lipid in an amount of at most 10% w/w, preferably at most 5% w/w, more preferably at most 2% w/w, and even more preferably at most 0.1% w/w.

The present inventors have found that it can be advantageous to control the mineral content to reach some of the desired properties of the BLG isolate powder.

In some preferred embodiments of the invention, the sum of the amounts of Na, K, Mg, and Ca of the BLG isolate powder is at most 10 mmol/g protein. Preferably, the sum of the amounts of Na, K, Mg, and Ca of the BLG isolate powder is at most 6 mmol/g protein, more preferably at most 4 mmol/g protein, even more preferably at most 2 mmol/g protein.

In other preferred embodiments of the invention, the the sum of the amounts of Na, K, Mg, and Ca of the BLG isolate powder is at most 1 mmol/g protein. Preferably, the sum of the amounts of Na, K, Mg, and Ca of the BLG isolate powder is at most 0.6 mmol/g protein, more preferably at most 0.4 mmol/g protein, even more preferably at most 0.2 mmol/g protein, and most preferably at most 0.1 mmol/g protein.

In other preferred embodiments of the invention, the sum of the amounts of Mg and Ca of the BLG isolate powder is at most 5 mmol/g protein. Preferably, the sum of the amounts of Mg and Ca of the BLG isolate powder is at most 3 mmol/g protein, more preferably at most 1.0 mmol/g protein, even more preferably at most 0.5 mmol/g protein.

In other preferred embodiments of the invention, the sum of the amounts of Mg and Ca of the BLG isolate powder is at most 0.3 mmol/g protein. Preferably, the sum of the amounts of Mg and Ca of the BLG isolate powder is at most 0.2 mmol/g protein, more preferably at most 0.1 mmol/g protein, even more preferably at most 0.03 mmol/g protein, and most preferably at most 0.01 mmol/g protein.

The inventors have found that it is possible to use low phosphorus/low potassium variants of the BLG isolate powder that are particularly useful to patients with kidney diseases. To make such a product, the BLG isolate powder has to have an equally low content of phosphorus and potassium.

Thus, in some preferred embodiments of the invention, the BLG isolate powder has a total content of phosphorus of at most 100 mg phosphorus per 100 g protein. Preferably, the BLG isolate powder has a total content of at most 80 mg phosphorus per 100 g protein. More preferably, the BLG isolate powder has a total content of at most 50 mg phosphorus per 100 g protein. Even more preferably, the BLG isolate powder has a total content of phosphorus of at most 20 mg phosphorus per 100 g protein. The BLG isolate powder has a total content of phosphorus of at most 5 mg phosphorus per 100 g protein.

In some preferred embodiments of the invention, the BLG isolate powder comprises at most 600 mg potassium per 100 g protein. More preferably, the BLG isolate powder comprise at most 500 mg potassium per 100 g protein. More preferably, the BLG isolate powder comprises at most 400 mg potassium per 100 g protein. More preferably, the BLG isolate powder comprises at most 300 mg potassium per 100 g protein. Even more preferably, the BLG isolate powder at most 200 mg potassium per 100 g protein. Even more preferably, the BLG isolate powder comprises at most 100 mg potassium per 100 g protein. Even more preferably, the BLG isolate powder comprises at most 50 mg potassium per 100 g protein and even more preferably, the BLG isolate powder comprises at most 10 mg potassium per 100 g protein.

The content of phosphorus relates to the total amount of elemental phosphorus of the composition in question and is determined according to Example 1.19. Similarly, the content of potassium relates to the total amount of elemental potassium of the composition in question and is determined according to Example 1.19.

In some preferred embodiments of the invention, the BLG isolate powder comprises at most 100 mg phosphorus/100 g protein and at most 700 mg potassium/100 g protein, preferably at most 80 mg phosphorus/100 g protein and at most 600 mg potassium/100 g protein, more preferably at most 60 mg phosphorus/100 g protein and at most 500 mg potassium/100 g protein, more preferably at most 50 mg phosphorus/100 g protein and at most 400 mg potassium/100 g protein, or more preferably at most 20 mg phosphorus/100 g protein and at most 200 mg potassium/100 g protein, or even more preferably at most 10 mg phosphorus/100 g protein and at most 50 mg potassium/100 g protein. In some preferred embodiments of the invention the BLG isolate powder comprises at most 100 mg phosphor/100 g protein and at most 340 mg potassium/100 g protein.

The low phosphorus and/or low potassium compositions according to the present invention may be used as a food ingredient for the production of a food product for patients groups that have a reduced kidney function.

The present inventors have found that for some applications, e.g. acidic food products and particularly acidic beverages, it is particularly advantageous to have an acidic BLG isolate powder having a pH of at most 4.9 and even more preferably at most 4.3. This is especially true for high protein, transparent acidic beverages.

In the context of the present invention, a transparent liquid has a turbidity of at most 200 NTU measured according to Example 1.7.

Thus, in some preferred embodiments of the invention, the BLG isolate powder has a pH in the range of 2-4.9. Preferably, the BLG isolate powder has a pH in the range of 2.5-4.7, more preferably 2.8-4.3, even more preferably 3.2-4.0, and most preferably 3.4-3.9. Alternatively, but also preferred, the BLG isolate powder may have a pH in the range of 3.6-4.3.

The present inventors have found that for some applications, e.g. pH-neutral food products and particularly pH-neutral beverages, it is particularly advantageous to have a pH-neutral BLG isolate powder. This is especially true for high protein, transparent or opaque pH-neutral beverages.

Thus, in some preferred embodiments of the invention, BLG isolate powder has a pH in the range of 6.1-8.5. Preferably, the powder has a pH in the range of 6.1-8.5, more preferably 6.2-8.0, even more preferably 6.3-7.7, and most preferably 6.5-7.5.

In other preferred embodiments of the invention, BLG isolate powder has a pH in the range of 5.0-6.0. Preferably, the powder has a pH in the range of 5.1-5.9, more preferably 5.2-5.8, even more preferably 5.3-5.7, and most preferably 5.4-5.6.

Advantageously, the BLG isolate powder used in the present invention may have bulk density of at least 0.20 g/cm³, preferably at least 0.30 g/cm³, more preferably at least 0.40 g/cm³, even more preferably at least 0.45 g/cm³, even more preferably at least 0.50 g/cm³, and most preferably at least 0.6 g/cm³.

Low density powders such as freeze-dried BLG isolates are fluffy and easily drawn into the air of the production site during use. This is problematic as it increases the risk of cross-contamination of the freeze-dried powder to other foods products and a dusty environment is known to be a cause of hygiene issues. In extreme cases, a dusty environment also increases the risk of dust explosions.

The high density variants of the present invention are easier to handle and less prone to flow into the surrounding air.

An additional advantage of the high density variants of the present invention is that they take up less space during transportation and thereby increase weight of BLG isolate powder that can be transported in one volume unit.

Yet an advantage of the high density variants of the present invention is that they are less prone to segregation when used in powder mixtures with other powdered food ingredients, such as e.g. powdered sugar (bulk density of approx. 0.56 g/cm³), granulated sugar (bulk density of approx. 0.71 g/cm³), powdered citric acid (bulk density of approx. 0.77 g/cm³).

The BLG isolate powder of the present invention may have bulk density in the range of 0.2-1.0 g/cm³, preferably in the range of 0.30-0.9 g/cm³, more preferably in the range of 0.40-0.8 g/cm³, even more preferably in the range of 0.45-0.75 g/cm³, even more preferably in the range of 0.50-0.75 g/cm³, and most preferably in the range of 0.6-0.75 g/cm³.

The bulk density of a powder is measured according to Example 1.17.

The present inventors have found that it is advantageous to maintain the native conformation of BLG and have seen indications that increased unfolding of BLG gives rise to an increased level of drying mouthfeel when the BLG is used for acidic beverages.

The intrinsic tryptophan fluorescence emission ratio (I330/I350) is a measure of degree of unfolding of BLG and the inventors have found that at high intrinsic tryptophan fluorescence emission ratios, which correlate with low or no unfolding of BLG, less drying mouthfeel was observed. The intrinsic tryptophan fluorescence emission ratio (I330/I350) is measured according to Example 1.1.

In some preferred embodiments of the invention, the BLG isolate powder has an intrinsic tryptophan fluorescence emission ratio (I330/I350) of at least 1.11.

In some preferred embodiments of the invention, the BLG isolate powder has an intrinsic tryptophan fluorescence emission ratio (I330/I350) of at least 1.12, preferably at least 1.13, more preferably at least 1.15, even more preferably at least 1.17, and most preferably at least 1.19.

If BLG isolate powder contains considerable amounts of non-protein matter it is preferred to isolate the protein fraction before measuring the intrinsic tryptophan fluorescence emission ratio. Thus in some preferred embodiments of the invention, the protein fraction of the BLG isolate powder has an intrinsic tryptophan fluorescence emission ratio of at least 1.11.

In some preferred embodiments of the invention, the protein fraction of the BLG isolate powder has an intrinsic tryptophan fluorescence emission ratio (I330/I350) of at least 1.12, preferably at least 1.13, more preferably at least 1.15, even more preferably at least 1.17, and most preferably at least 1.19.

The protein fraction can e.g. be separated from the BLG isolate powder by dissolving the BLG isolate powder in demineralised water and subjecting the solution to dialysis or ultrafiltration-based diafiltration using a filter that retains the protein. If the BLG isolate powder contains interfering levels of lipid such lipid can e.g. be removed by microfiltration. Steps of microfiltration and ultrafiltration/diafiltration can be combined to remove both lipid and small molecules from the protein fraction.

It is often preferred that a substantial amount of the BLG of the BLG isolate powder is non-aggregated BLG. Preferably at least 50% of the BLG is non-aggregated BLG. More preferably at least at least 80% of the BLG is non-aggregated BLG. Even more preferred at least 90% of the BLG is non-aggregated BLG. Most preferred, at least 95% of the BLG is non-aggregated BLG. Even more preferred approx. 100% of the BLG of the BLG isolate powder is non-aggregated BLG.

In some preferred embodiments of the invention, the BLG isolate powder has a degree of protein denaturation of at most 10%, preferably at most 8%, more preferably at most 6%, even more preferably at most 3%, even more preferably at most 1%, and most preferably at most 0.2%.

However, it may also be preferred that the BLG isolate powder has a significant level of protein denaturation, e.g. if an opaque beverage is desired. Thus, in other preferred embodiments of the invention, the BLG isolate powder has a degree of protein denaturation of at least 11%, preferably at least 20%, more preferably at least 40%, even more preferably at least 50%, even more preferably at least 75%, and most preferably at least 90%.

If BLG isolate powder has a significant level of protein denaturation it is often preferred to keep a low level of insoluble protein matter, i.e. precipitated protein matter that would settle in a beverage during storage. The level of insoluble matter is measure according to Example 1.10.

In some preferred embodiments of the invention the BLG isolate powder comprises at most 20% w/w insoluble protein matter, preferably at most 10% w/w insoluble protein matter, more preferably at most 5% w/w insoluble protein matter, even more preferred at most 3% w/w insoluble protein matter, and most preferred at most 1% w/w insoluble protein matter. It may even be preferred that the BLG isolate powder does not contain any insoluble protein matter at all.

The present inventors have found that the heat-stability at pH 3.9 of a BLG isolate powder is a good indicator for its usefulness for transparent high protein beverages. The heat-stability at pH 3.9 is measured according to Example 1.2.

It is particularly preferred that the BLG isolate powder has a heat-stability at pH 3.9 of at most 200 NTU, preferably at most 100 NTU, more preferred at most 60 NTU, even more preferred at most 40 NTU, and most preferred at most 20 NTU. Even better heat-stabilities are possible and the BLG isolate powder preferably has a heat-stability at pH 3.9 of at most 10 NTU, preferably at most 8 NTU, more preferred at most 4 NTU, even more preferred at most 2 NTU.

The content of microorganisms of the BLG isolate powder is preferably kept to a minimum. However, it is a challenge to obtain both a high degree of protein nativeness and a low content of microorganism as processes for microbial reduction tend to lead to protein unfolding and denaturation. The present invention makes it possible to obtain a very low content of microorganism while at the same time maintain a high level of the nativeness of BLG.

Thus, in some preferred embodiments of the invention, the BLG isolate powder contains at most 15000 colony-forming units (CFU)/g. Preferably, the BLG isolate powder contains at most 10000 CFU/g. More preferably, the BLG isolate powder contains at most 5000 CFU/g. Even more preferably, the BLG isolate powder contains at most 1000 CFU/g. Even more preferably, the BLG isolate powder contains at most 300 CFU/g. Most preferably, the BLG isolate powder contains at most 100 CFU/g such as e.g. at most 10 CFU/g. In a particularly preferred embodiment the powder is sterile. A sterile BLG isolate powder may e.g. be prepared by combining several physical microbial reduction processes during the production of the BLG isolate powder, such as e.g. microfiltration and heat-treatment at acidic pH.

In some preferred embodiments of the invention, the BLG isolate powder has a pH in the range of i) 2-4.9, ii) 6.1-8.5, or iii) 5.0-6.0 and comprises:
total protein in an amount of at least 30% w/w, preferably at least 80% w/w, and even more preferably at least 90% w/w
beta-lactoglobulin (BLG) in an amount of at least 85% w/w relative to total protein, preferably at least 90% w/w,
water in an amount of at most 6% w/w,
lipid in an amount of at most 2% w/w, preferably at most 0.5% w/w,

said BLG isolate powder having:
an intrinsic tryptophan fluorescence emission ratio (I330/I350) of at least 1.11,
a degree of protein denaturation of at most 10%, and
a heat-stability at pH 3.9 of at most 200 NTU.

In some preferred embodiments of the invention, the BLG isolate powder has a pH in the range of i) 2-4.9 or ii) 6.1-8.5 and comprises:
total protein in an amount of at least 30% w/w, preferably at least 80% w/w, and even more preferably at least 90% w/w
beta-lactoglobulin (BLG) in an amount of at least 85% w/w relative to total protein, preferably at least 90% w/w, and more preferably at least 94% w/w relative to total protein
water in an amount of at most 6% w/w,
lipid in an amount of at most 2% w/w, preferably at most 0.5% w/w,

said BLG isolate powder having:
an intrinsic tryptophan fluorescence emission ratio (I330/I350) of at least 1.11,
a degree of protein denaturation of at most 10%, preferably at most 5%, and
a heat-stability at pH 3.9 of at most 70 NTU, preferably at most 50 NTU and even more preferably at most 40 NTU.

In some preferred embodiments of the invention the BLG isolate powder has a pH in the range of i) 2-4.9 or ii) 6.1-8.5 and comprises:
total protein in an amount of at least 30% w/w,
beta-lactoglobulin (BLG) in an amount of at least 85% w/w relative to total protein, preferably at least 90% w/w,
water in an amount of at most 6% w/w,

said BLG isolate powder having:
a bulk density of at least 0.2 g/cm³,
an intrinsic tryptophan fluorescence emission ratio (I330/I350) of at least 1.11,
a degree of protein denaturation of at most 10%, and
a heat-stability at pH 3.9 of at most 200 NTU.

In other preferred embodiments of the invention, the BLG isolate powder has a pH in the range of 2-4.9 and comprises:
total protein in an amount of at least 80% w/w, preferably at least 90% w/w, and even more preferably at least 94% w/w
beta-lactoglobulin (BLG) in an amount of at least 85% w/w relative to total protein, preferably at least 90% w/w, and even more preferably at least 94% w/w relative to total protein,
water in an amount of at most 6% w/w,
lipid in an amount of at most 2% w/w, preferably at most 0.5% w/w,

said BLG isolate powder having:
a bulk density of at least 0.2 g/cm³, preferably at least 0.3 g/cm³, and more preferably at least 0.4 g/cm³,
an intrinsic tryptophan fluorescence emission ratio (I330/I350) of at least 1.11,
a degree of protein denaturation of at most 10%, preferably at most 5%, and more preferably at most 2%, and
a heat-stability at pH 3.9 of at most 50 NTU, preferably at most 30 NTU and even more preferably at most 10 NTU.

In yet other preferred embodiments of the invention, the BLG isolate powder has a pH in the range of 6.1-8.5 and comprises:
total protein in an amount of at least 80% w/w, preferably at least 90% w/w, and even more preferably at least 94% w/w
beta-lactoglobulin (BLG) in an amount of at least 85% w/w relative to total protein, preferably at least 90% w/w, and even more preferably at least 94% w/w relative to total protein,
water in an amount of at most 6% w/w,
lipid in an amount of at most 2% w/w, preferably at most 0.5% w/w,

said BLG isolate powder having:
a bulk density of at least 0.2 g/cm³, preferably at least 0.3 g/cm³, and more preferably at least 0.4 g/cm³,
a degree of protein denaturation of at most 10%, preferably at most 5%, and more preferably at most 2%, and
a heat-stability at pH 3.9 of at most 50 NTU, preferably at most 30 NTU, and even more preferably at most 10 NTU.

In further preferred embodiments of the invention, the BLG isolate powder has a pH in the range of 6.1-8.5 and comprises:
total protein in an amount of at least 80% w/w, preferably at least 90% w/w, and even more preferably at least 94% w/w
beta-lactoglobulin (BLG) in an amount of at least 85% w/w relative to total protein, preferably at least 90% w/w, and even more preferably at least 94% w/w relative to total protein,
water in an amount of at most 6% w/w,
lipid in an amount of at most 2% w/w, preferably at most 0.5% w/w,

said BLG isolate powder having:
a bulk density of at least 0.2 g/cm³, preferably at least 0.3 g/cm³, and more preferably at least 0.4 g/cm³,
a degree of protein denaturation of at most 10%, preferably at most 5%, and more preferably at most 2%, and
a heat-stability at pH 3.9 of at most 50 NTU, preferably at most 30 NTU, and even more preferably at most 10 NTU.

In further preferred embodiments of the invention, the BLG isolate powder has a pH in the range of 5.0-6.0 and comprises:
total protein in an amount of at least 80% w/w, preferably at least 90% w/w, and even more preferably at least 94% w/w,
beta-lactoglobulin (BLG) in an amount of at least 85% w/w relative to total protein, preferably at least 90% w/w, and even more preferably at least 94% w/w relative to total protein,
water in an amount of at most 6% w/w,
lipid in an amount of at most 2% w/w, preferably at most 0.5% w/w,

said BLG isolate powder having:
a bulk density of at least 0.2 g/cm³, preferably at least 0.3 g/cm³, and more preferably at least 0.4 g/cm³,
a degree of protein denaturation of at most 10%, preferably at most 5%, and more preferably at most 2%,
a heat-stability at pH 3.9 of at most 50 NTU, preferably at most 30 NTU, and even more preferably at most 10 NTU, and
preferably, a BLG crystallinity of less than 10%.

The BLG isolate powder containing BLG in an amount of at least 85% w/w relative to total protein, is typically provided by a method comprising the steps of:
a) providing a liquid BLG isolate having
   i) a pH in the range of 2-4.9,
   ii) a pH of in the range of 6.1-8.5, or
   iii) a pH of in the range of 5.0-6.0
   said liquid BLG isolate containing BLG in an amount of at least 85 w/w relative to total protein,
b) optionally, subjecting the liquid BLG isolate to a physical microbial reduction,
c) drying the liquid BLG isolate, preferably by spray-drying.

The BLG isolate is preferably prepared from mammal milk, and preferably from ruminant milk such as e.g. milk from cow, sheep, goat, buffalo, camel, llama, mare and/or deer. Protein derived from bovine milk is particularly preferred. The BLG is therefore preferably bovine BLG.

The liquid BLG isolate may be provided in a number of different ways.

Typically, the provision of the liquid BLG isolate involves, or even consists of, isolating BLG from a whey protein feed to provide a BLG-enriched composition by one or more of the following methods:
crystallisation or precipitation of BLG by salting-in,
crystallisation or precipitation of BLG of BLG by salting-out,
ion exchange chromatography, and
fractionation of whey proteins by ultrafiltration.

A particularly preferred way of providing the BLG-enriched composition is by crystallisation of BLG, preferably by salting-in or alternatively by salting-out.

The whey protein feed is preferably a WPC, a WPI, an SPC, an SPI, or a combination thereof.

The term “whey protein feed” pertains to the composition from which the BLG-enriched composition and subsequently the liquid BLG isolate are derived.

In some embodiments of the invention, the preparation of the BLG-enriched composition includes, or even consist of, high salt BLG crystallisation in the pH range 3.6-4.0 according to U.S. Pat. No. 2,790,790 A1.

In other embodiments of the invention the preparation of the BLG-enriched composition includes, or even consists of, the method described by de Jongh et al (Mild Isolation Procedure Discloses New Protein Structural Properties of β-Lactoglobulin, J Dairy Sci., vol. 84(3), 2001, pages 562-571) or by Vyas et al (Scale-Up of Native β-Lactoglobulin Affinity Separation Process, J. Dairy Sci. 85:1639-1645, 2002).

However, in particularly preferred embodiments of the invention, the BLG-enriched composition is prepared by crystallisation at pH 5-6 under salting-in conditions as described in the PCT application PCT/EP2017/084553, which is incorporated herein by reference for all purposes.

In some preferred embodiments of the invention, the BLG-enriched composition is an edible BLG composition according to PCT/EP2017/084553 containing at least 90% BLG relative to total protein and preferably containing BLG crystals.

If it does not already have the required characteristics to be used as liquid BLG isolate, the BLG-enriched composition which has been isolated from whey protein feed may be subjected to one or more steps selected from the group of:
demineralisation,
addition of minerals
dilution,
concentration,
physical microbioal reduction, and
pH adjustment

as part of providing the liquid BLG isolate.

Non-limiting examples of demineralisation include e.g. dialysis, gel filtration, UF/diafiltration, NF/diafiltration, and ion exchange chromatography.

Non-limiting examples of addition of minerals include addition of soluble, food acceptable salts, such as e.g. salts of Na, K, Ca, and/or Mg. Such salts may e.g. be phosphate-salts, chloride salts or salts of food acids, such as e.g. citrate salt or lactate salt. The minerals may be added in solid, suspended, or dissolved form.

Non-limiting examples of dilution include e.g. addition of liquid diluent such as water, demineralised water, or aqueous solutions of minerals, acids or bases.

Non-limiting examples of concentration include e.g. evaporation, reverse osmosis, nanofiltration, ultrafiltration and combinations thereof.

If the concentration has to increase the concentration of protein relative to total solids, it is preferred to use concentration steps such as ultrafiltration or alternatively dialysis. If the concentration does not have to increase the concentration of protein relative to total solids, methods such as e.g. evaporation, nanofiltration and/or reverse osmosis can be useful.

Non-limiting examples of physical microbial reduction include e.g. heat-treatment, germ filtration, UV radiation, high pressure treatment, pulsed electric field treatment, and ultrasound. These methods are well-known to the person skilled in the art.

Non-limiting examples of pH adjustment include e.g. addition of bases and/or acids, and preferably food acceptable bases and/or acids. It is particularly preferred to employ acids and/or bases that are capable of chelating divalent metal cations. Examples of such acids and/or bases are citric acid, citrate salt, EDTA, lactic acid, lactate salt, phosphoric acid, phosphate salt, and combinations thereof.

In some preferred embodiments of the present invention, the liquid solution has a colour value delta b* in the range of −0.10 to +0.51 at the CIELAB colour scale, particularly if the preparation has a turbidity of at most 200 NTU, and more preferably at most 40 NTU.

In other preferred embodiments of the invention, the liquid solution has a colour value delta b* in the range of 0.0 to 0.40 at the CIELAB colour scale, preferably in the range of +0.10 to +0.25.

The liquid solution of the present invention may comprise other macronutrients than proteins. In some embodiments of the invention the liquid solution furthermore comprises carbohydrates. The total carbohydrate content in the liquid solution of the invention depends on the intended use of the final heat-treated beverage preparation.

In some preferred embodiments of the invention, the liquid solution furthermore comprises at least one source of carbohydrate. In one exemplary embodiment, the at least one source of carbohydrate is selected from the group consisting of: sucrose, maltodextrin, corn syrup solids, sucromalt, glucose polymers, corn syrup, modified starches, resistant starches, rice-derived carbohydrates, isomaltulose, white sugar, glucose, fructose, lactose, galactose, maltose, dextrose, high fructose corn syrup, honey, sugar alcohols, fructooligosaccharides, soy fiber, corn fiber, guar gum, konjac flour, polydextrose, Fibersol, and combinations thereof.

In some preferred embodiments the liquid solution furthermore comprises carbohydrates in a range between 0 to 95% of the total energy content of the liquid solution, preferably in a range between 10 to 85% of the total energy content of the liquid solution, preferably in a range between 20 to 75% of the total energy content of the liquid solution or preferably in a range between 30 to 60% of the total energy content of the liquid

Even lower carbohydrate content is often preferred, thus in some preferred embodiments of the invention preferably in a range between 0 to 30% of the total energy content of the preparation more preferably in a range between 0 to 20% of the total energy content of the preparation even more preferably in a range between 0 to 10% of the total energy content of the preparation.

In one embodiment of the invention the liquid solution furthermore comprises at least one additional ingredient selected from the group consisting of vitamins, flavouring agent, minerals, sweeteners, antioxidants, food acid, lipids, carbohydrate, prebiotics, probiotics and non-whey protein.

In one embodiment of the invention, the liquid solution furthermore comprises at least one high intensity sweetener. In one embodiment, the at least one high intensity sweetener is selected from the group consisting of aspartame, cyclamate, sucralose, acesulfame salt, neotame, saccharin, Stevia extract, a steviol glycoside such as e.g. rebaudioside A, or a combination thereof. In some embodiments of the invention, it is particularly preferred that the sweetener comprises or even consists of one or more high intensity sweeteners (HIS).

HIS are both found among both natural and artificial sweeteners and typically have a sweetening intensity of at least 10 times that of sucrose.

If used, the total amount of HIS is typically in the range of 0.01-2% w/w. For example, the total amount of HIS may be in the range of 0.05-1.5% w/w. Alternatively, the total amount of HIS may be in the range of 0.1-1.0% w/w.

The choice of the sweetener may depend on the beverage to be produced, e.g. high-intensity sugar sweeteners (e.g. aspartame, acesulfame-K or sucralose) may be used in beverage where no energy contribution from the sweetener is desired, whereas for beverages having a natural profile natural sweeteners (e.g. steviol glycosides, sorbitol or sucrose) may be used.

It may furthermore be preferred that the sweetener comprises or even consists of one or more polyol sweetener(s). Non-limiting examples of useful polyol sweeteners are maltitol, mannitol, lactitol, sorbitol, inositol, xylitol, threitol, galactitol or combinations thereof. If used, the total amount of polyol sweetener is typically in the range of 1-20% w/w. For example, the total amount of polyol sweetener may be in the range of 2-15% w/w. Alternatively, the total amount of polyol sweetener may be in the range of 4-10% w/w.

The liquid solution of the present invention may comprise other macronutrients than proteins. In some embodiments of the invention the liquid solution furthermore comprises lipids. The total lipid content in the final heat-treated beverage preparation of the invention depends on the intended use of the heat-treated beverage preparation.

In some preferred embodiments of the invention, the liquid solution has a lipid content between 0 to 50% of the total energy content of the liquid solution, or preferably in a range between 0 to 45% of the total energy content of the liquid solution, or preferably in a range between 0 to 30% of the total energy content of the liquid solution or preferably in a range between 0 to 20% of the total energy content of the liquid solution or preferably in a range between 0 to 10% of the total energy content of the liquid solution or preferably in a range between 0 to 5% of the total energy content of the liquid solution.

The amount of lipid is determined according to ISO 1211:2010 (Determination of Fat Content—Röse-Gottlieb Gravimetric Method).

The present inventors have found that it can be advantageous to control the mineral content to reach some of the desired properties of the packaged heat-treated beverage preparation.

In some embodiments of the invention the packaged heat-treated beverage preparation comprises a plurality of minerals. In one exemplary embodiment, the liquid solution comprises at least four minerals. In one embodiment the four minerals are sodium, potassium, magnesium and calcium.

The present inventors have surprisingly found that when a BLG isolate is used as defined herein and in example 2, heat-treated beverage preparations having a high mineral concentration can be produced, without compromising the viscosity. This provides the possibility that packaged heat-treated beverage preparations can be produced having a high mineral content and that beverages that are nutritionally complete nutritional supplements or nutritionally incomplete supplements can be produced.

In some preferred embodiments of the invention the sum of the amounts of Na, K, Mg and Ca is within the range of 0 to 750 mM in the liquid solution, preferably within the range of 100-600 mM or preferably within the range of 200-500 mM.

In some preferred embodiments of the invention the sum of the amounts of Na, K, Mg and Ca is at most 750 mM in the liquid solution.

In other preferred embodiments of the invention the sum of the amounts of Na, K, Mg and Ca is at most 600 mM in the liquid solution, preferably at most 500 mM, or preferably at most 400 mM, or preferably at most 300 mM, or preferably at most 200 mM, preferably at most 170 mM, most preferably at most 150 mM, or preferably at most 130 mM, or preferably at most 100 mM or preferably at most 80 mM or preferably at most 60 mM or preferably at most 40 mM or preferably at most 30 mM or preferably at most 20 mM or preferably at most 10 mM or preferably at most 5 mM or preferably at most 1 mM.

In another exemplary embodiment, the liquid solution comprises a plurality of minerals selected from the group consisting of: Calcium, Iodine, Zinc, Copper, Chromium, Iron, Phosphorus, Magnesium, Selenium, Manganese, Molybdenum, Sodium, Potassium, and combinations thereof.

In some preferred embodiments of the present invention the liquid solution comprises at most 150 mM KCl and at most 150 mM CaCl₂), or the liquid solution comprises at most 130 mM KCl and at most 130 mM CaCl₂) or the liquid solution comprises at most 110 mM KCl and at most 110 mM CaCl₂) or the liquid solution comprises at most 100 mM KCl and at most 100 mM CaCl₂) or preferably the liquid solution comprises at most 80 mM KCl and at most 80 mM CaCl₂) or preferably the liquid solution comprises at most 50 mM KCl and at most 50 mM CaCl₂) or preferably the liquid solution comprises at most 40 mM KCl and at most 40 mM CaCl₂).

In other preferred embodiments of the invention the liquid solution is a low mineral beverage.

In the context of the present invention the term “low mineral” pertains to a composition, e.g. a liquid, beverage, a powder or another food product, that has at least one, preferably two, and even more preferably all, of the following:
an ash content of at most 1.2% w/w relative to total solids,
a total content of calcium and magnesium of at most 0.3% w/w relative to total solids,
a total content of sodium and potassium of at most 0.10% w/w relative to total solids,
a total content of phosphorus of at most 100 mg phosphorus per 100 g protein.

Preferably, a low mineral composition has at least one, preferably two or more, and even more preferably all, of the following:
an ash content of at most 0.7% w/w relative to total solids,
a total content of calcium and magnesium of at most 0.2% w/w relative to total solids,
a total content of sodium and potassium of at most 0.08% w/w relative to total solids,
a total content of phosphorus of at most 80 mg phosphorus per 100 g protein.

Even more preferably, a low mineral composition has at least one, preferably two or more, and even more preferably all, of the following:
an ash content of at most 0.5% w/w relative to total solids,
a total content of calcium and magnesium of at most 0.15% w/w relative to total solids,
a total content of sodium and potassium of at most 0.06% w/w relative to total solids,
a total content of phosphorus of at most 50 mg phosphorus per 100 g protein.

It is particularly preferred that a low mineral composition has the following:
an ash content of at most 0.5% w/w relative to total solids,
a total content of calcium and magnesium of at most 0.15% w/w relative to total solids,
a total content of sodium and potassium of at most 0.06% w/w relative to total solids,
a total content of phosphorus of at most 50 mg phosphorus per 100 g protein.

The present inventors have found that the present invention makes it possible to prepare a packaged heat-treated beverage preparation having a very low content of phosphorus and other minerals such as Potassium, which is advantageous for patients suffering from kidney diseases or otherwise having a reduced kidney function.

The liquid solution is preferably a low phosphorus solution.

The liquid solution is preferably a low Potassium solution.

The liquid solution is preferably low phosphorus and a low Potassium solution.

In the context of the present invention the term “low phosphorus” pertains to a composition, e.g. a liquid, a powder or another food product, that has a total content of phosphorus of at most 100 mg phosphorus per 100 g protein. Preferably, a low phosphorus composition has a total content of at most 80 mg phosphorus per 100 g protein. More preferably, a low phosphorus composition may have a total content of at most 50 mg phosphorus per 100 g protein. Even more preferably, a low phosphorus composition may have a total content of phosphorus of at most 20 mg phosphorus per 100 g protein. Even more preferably, a low phosphorus composition may have a total content of phosphorus of at most 5 mg phosphorus per 100 g protein. Low phosphorus compositions according to the present invention may be used as a food ingredient for the production of a food product for patient groups that have a reduced kidney function.

Thus, in some particularly preferred embodiments of the invention the liquid solution comprises at most 80 mg phosphorus per 100 g protein. Preferably, the liquid solution comprises at most 30 mg phosphorus per 100 g protein. More preferably, the liquid solution comprises at most 20 mg phosphorus per 100 g protein. Even more preferably, the liquid solution comprises at most 10 mg phosphorus per 100 g protein. Most preferably, the liquid solution comprises at most 5 mg phosphorus per 100 g protein.

The content of phosphorus relates to the total amount of elemental phosphorus of the composition in question and is determined according to Example 1.19.

In the context of the present invention the term “low potassium” pertains to a composition, e.g. a liquid, a powder or another food product, that has a total content of potassium of at most 700 mg potassium per 100 g protein. Preferably, a low phosphorus composition has a total content of at most 600 mg potassium per 100 g protein. More preferably, a low potassium composition may have a total content of at most 500 mg potassium per 100 g protein. More preferably, a low potassium composition may have a total content of potassium of at most 400 mg potassium per 100 g protein. More preferably, a low potassium composition may have a total content of potassium of at most 300 mg potassium per 100 g protein. Even more preferably, a low potassium composition may have a total content of potassium of at most 200 mg potassium per 100 g protein. Even more preferably, a low potassium composition may have a total content of potassium of at most 100 mg potassium per 100 g protein. Even more preferably, a low potassium composition may have a total content of potassium of at most 50 mg potassium per 100 g protein and even more preferably, a low potassium composition may have a total content of potassium of at most 10 mg potassium per 100 g protein.

Low potassium compositions according to the present invention may be used as a food ingredient for the production of a food product for patient groups that have a reduced kidney function.

Thus, in some particularly preferred embodiments of the invention the liquid solution comprises at most 600 mg potassium per 100 g protein. More preferably, the liquid solution comprises at most 500 mg potassium per 100 g protein. More preferably, the liquid solution comprises at most 400 mg potassium per 100 g protein. More preferably, the liquid solution comprises at most 300 mg potassium per 100 g protein. Even more preferably, the liquid solution comprises at most 200 mg potassium per 100 g protein. Even more preferably, the liquid solution comprises at most 100 mg potassium per 100 g protein. Even more preferably, the liquid solution comprises at most 50 mg potassium per 100 g protein and even more preferably, the liquid solution comprises at most 10 mg potassium per 100 g protein

The content of potassium relates to the total amount of elemental phosphorus of the composition in question and is determined according to Example 1.19.

In some preferred embodiments of the invention the liquid solution comprises at most 100 mg phosphorus/100 g protein and at most 700 mg potassium/100 g protein, preferably at most 80 mg phosphorus/100 g protein and at most 600 mg potassium/100 g protein, more preferably at most 60 mg phosphorus/100 g protein and at most 500 mg potassium/100 g protein, more preferably at most 50 mg phosphorus/100 g protein and at most 400 mg potassium/100 g protein, or more preferably at most 20 mg phosphorus/100 g protein and at most 200 mg potassium/100 g protein, or even more preferably at most 10 mg phosphorus/100 g protein and at most 50 mg potassium/100 g protein. In some preferred embodiments of the invention the packaged, heat-treated beverage preparation comprises at most 100 mg phosphor/100 g protein and at most 340 mg potassium/100 g protein.

The liquid solution comprising low amounts of phosphorus and Potassium may advantageously be supplemented with carbohydrates and lipids, the heat-treated beverage preparation preferably furthermore comprises a total amount of carbohydrates in a range between 30-60% of the total energy content of the liquid solution, preferably in a range between 35-50E % and a total amount of lipid in the range of 20-60% of the total energy content, preferably in a range between 30-50E %.

In one embodiment of the invention the liquid solution comprises a plurality of vitamins. In one exemplary embodiment, the liquid solution comprises at least ten vitamins. In one exemplary embodiment, the liquid solution comprises a plurality of vitamins selected from the group consisting of: Vitamin A, vitamin B1, vitamin B2, vitamin B3, vitamin B5, vitamin B6, vitamin B7, vitamin B9, vitamin B12, vitamin C, vitamin D, vitamin K, Riboflavin, pantothenic Acid, vitamin E, thiamin, niacin, folic acid, biotin, and combinations thereof.

In one embodiment of the invention, the liquid solution comprises a plurality of vitamins and a plurality of minerals.

In some preferred embodiments of the present invention the liquid solution contains one or more food acids selected from the group consisting of citric acid, malic acid, tartaric acid, acetic acid, benzoic acid, butyric acid, lactic acid, fumaric acid, succinic acid, ascorbic acid, adipic acid, phosphoric acid, and mixtures thereof.

In an embodiment of the present invention, the liquid solution furthermore comprises a flavor selected from the group consisting of salt, flavorings, flavor enhancers and/or spices. In a preferred embodiment of the invention the flavor comprises chocolate, cocoa, lemon, orange, lime, strawberry, banana, forest fruit flavor or combinations thereof. The choice of flavor may depend on the beverage to be produced.

An aspect of the invention pertains to the use of a protein solution comprising a total amount of protein of 3 to 35% w/w relative to the weight of the solution, wherein at least 90 w/w % of the protein is BLG for controlling the turbidity of a heat-treated acidic beverage preparation having a pH in the range of 2.0-4.7.

Another aspect of the invention pertains to the use of a protein solution comprising a total amount of protein of 2 to 45% w/w relative to the weight of the solution, wherein at least 90 w/w % of the protein is BLG for controlling the astringency of a heat-treated acidic beverage preparation having a pH in the range of 2.0-4.7.

Another aspect of the invention pertains to a packaged heat-treated beverage preparation as defined herein, for use in a method for the treatment of diseases associated with protein malabsorption.

Another aspect of the invention pertains to use of the packaged heat-treated beverage preparation as defined herein as a dietary supplement.

In a preferred embodiment of the invention the packaged heat-treated beverage preparation as defined herein is used as a dietary supplement and it is ingested before, during or after exercise.

In a preferred embodiment of the present invention the packaged, heat-treated beverage preparation has a pH in the range of 2.0-4.2, preferably 3.0-3.9 or preferably 3.2-3.7, the beverage comprising

a total amount of protein of 2 to 45% w/w relative to the weight of the beverage, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11
a lipid content of at most 5% of the total energy content of the preparation.

In a preferred embodiment of the present invention the packaged, heat-treated beverage preparation has a pH in the range of 2.0-4.2, preferably 3.0-3.9 or preferably 3.2-3.7, the beverage comprising

a total amount of protein of 2 to 10% w/w relative to the weight of the beverage, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11
a lipid content of at most 5% of the total energy content of the preparation.

In a preferred embodiment of the present invention the packaged, heat-treated beverage preparation has a pH in the range of 2.0-4.2, preferably 3.0-3.9 or preferably 3.2-3.7 the beverage comprising

a total amount of protein of 10 to 45% w/w relative to the weight of the beverage, preferably 10-35% w/w, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11
a lipid content of at most 5% of the total energy content of the preparation.

In a preferred embodiment of the present invention the packaged, heat-treated beverage preparation has a pH in the range of 32.0-4.2, preferably 3.0-3.9 or preferably 3.2-3.7, the beverage comprising

a total amount of protein of 2 to 45% w/w relative to the weight of the beverage, wherein at least 85% w/w of the protein is BLG, preferably at least 90% w/w, and
optionally, sweetener and/or flavour,
the packaged, heat-treated beverage preparation has a turbidity of at most 200 NTU, preferably at most 40 NTU.

In a preferred embodiment of the present invention the packaged, heat-treated beverage preparation has a pH in the range of 2.0-4.2, preferably 3.0-3.9 or preferably 3.2-3.7 the beverage comprising

a total amount of protein of 2 to 10% w/w relative to the weight of the beverage, wherein at least 85% w/w of the protein is BLG, preferably at least 90% w/w, and
optionally, sweetener and/or flavour,

the packaged, heat-treated beverage preparation has a turbidity of at most 200 NTU, preferably at most 40 NTU.

In a preferred embodiment of the present invention the packaged, heat-treated beverage preparation has a pH in the range of 2.0-4.2, preferably 3.0-3.9 or preferably 3.2-3.7 the beverage comprising

a total amount of protein of 10 to 45% w/w relative to the weight of the beverage, preferably 10-205 w/w, wherein at least 85% w/w of the protein is BLG, preferably at least 90% w/w, and
optionally, sweetener and/or flavour, the packaged, heat-treated beverage preparation has a turbidity of at most 200 NTU, preferably at most 40 NTU.

In a preferred embodiment of the present invention the packaged, heat-treated beverage preparation has a pH in the range of 2.0-4.2, preferably 3.0-3.9 or preferably 3.2-3.7, the beverage comprising

a total amount of protein of 2 to 45% w/w relative to the weight of the beverage, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11
the protein fraction of the beverage preparation has a colour value delta b* in the range of −0.10 to +0.51 at the CIELAB colour scale, wherein delta b*=b_{sample standardized to 6.0 w/w % protein}*−b_{demin. water}*, measured at room temperature.

In a preferred embodiment of the present invention the packaged, heat-treated beverage preparation has a pH in the range of 2.0-4.2, preferably 3.0-3.9 or preferably 3.2-3.7, the beverage comprising

a total amount of protein of 2 to 10% w/w relative to the weight of the beverage, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11
the protein fraction of the beverage preparation has a colour value delta b* in the range of −0.10 to +0.51 at the CIELAB colour scale, wherein delta b*=b_{sample standardized to 6.0 w/w % protein}*−b_{demin. water}*, measured at room temperature.

In a preferred embodiment of the present invention the packaged, heat-treated beverage preparation has a pH in the range of 2.0-4.2, preferably 3.0-3.9 or preferably 3.2-3.7, the beverage comprising

a total amount of protein of 10 to 45% w/w relative to the weight of the beverage, preferably 10-20% w/w, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11
the protein fraction of the beverage preparation has a colour value delta b* in the range of −0.10 to +0.51 at the CIELAB colour scale, wherein delta b*=b_{sample standardized to 6.0 w/w % protein}*−b_{demin. water}*, measured at room temperature.

In a preferred embodiment of the present invention the packaged, heat-treated beverage preparation has a pH in the range of 2.0-4.2, preferably 3.0-3.9 or preferably 3.2-3.7, the beverage comprising a total amount of protein of 2 to 45% w/w relative to the weight of the beverage, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11
the sum of the amounts of Na, K, Mg and Ca is at most 750 mM, preferably at most 400 mM, preferably at most 200 mM.

In a preferred embodiment of the present invention the packaged, heat-treated beverage preparation has a pH in the range of 2.0-4.2, preferably 3.0-3.9 or preferably 3.2-3.7, the beverage comprising

a total amount of protein of 2 to 10% w/w relative to the weight of the beverage, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11
the sum of the amounts of Na, K, Mg and Ca is at most 750 mM, preferably at most 400 mM, preferably at most 200 mM.

In a preferred embodiment of the present invention the packaged, heat-treated beverage preparation has a pH in the range of 2.0-4.2, preferably 3.0-3.9 or preferably 3.2-3.7, the beverage comprising

a total amount of protein of 10 to 45% w/w relative to the weight of the beverage, preferably 10-20% w/w, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11
the sum of the amounts of Na, K, Mg and Ca is at most 750 mM, preferably at most 400 mM, preferably at most 200 mM.

In a preferred embodiment of the present invention the packaged, heat-treated, opaque beverage preparation has a pH in the range of 3.0-4.7, preferably 3.9-4.6, more preferably 4.0-4.5, the beverage comprising:
a total amount of protein of 2 to 45% w/w relative to the weight of the beverage, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11 and/or
wherein the protein fraction has a degree of protein denaturation of at most 5% and/or
a lipid content of more than 5% of the total energy content of the preparation.

In a preferred embodiment of the present invention the packaged, heat-treated beverage preparation has a pH in the range of 3.0-4.7, preferably 3.9-4.6, more preferably 4.0-4.5, the beverage comprising:
a total amount of protein of 2 to 45% w/w relative to the weight of the beverage, wherein at least 85% w/w of the protein is BLG preferably at least 90% w/w, and
optionally, sweetener and/or flavour,

wherein:
the turbidity is more than 200 NTU, preferably more than 1000 NTU and/or
the viscosity is at most 200 cP.

In a preferred embodiment of the present invention the packaged, heat-treated, opaque beverage preparation has a pH in the range of 3.0-4.7, preferably 3.9-4.6, more preferably 4.0-4.5, the beverage comprising:
a total amount of protein of 2 to 10% w/w relative to the weight of the beverage, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11 and/or
wherein the protein fraction has a degree of protein denaturation of at most 5% and/or
a lipid content of more than 5% of the total energy content of the preparation.

In a preferred embodiment of the present invention the packaged, heat-treated beverage preparation has a pH in the range of 3.0-4.7, preferably 3.9-4.6, more preferably 4.0-4.5, the beverage comprising:
a total amount of protein of 2 to 10% w/w relative to the weight of the beverage, wherein at least 85% w/w of the protein is BLG preferably at least 90% w/w, and
optionally, sweetener and/or flavour,

wherein:
the turbidity is more than 200 NTU, preferably more than 1000 NTU and/or
the viscosity is at most 200 cP.

In a preferred embodiment of the present invention the packaged, heat-treated, opaque beverage preparation has a pH in the range of 3.0-4.7, preferably 3.9-4.6, more preferably 4.0-4.5, the beverage comprising
a total amount of protein of 10 to 45% w/w relative to the weight of the beverage, preferably 10-20% w/w, wherein at least 85% w/w of the protein is BLG, preferably at least 90% w/w, and
optionally, sweetener and/or flavour,

wherein:
the protein fraction of the beverage preparation has an intrinsic tryptophan fluorescence emission ratio (I330 nm/I350 nm) of at least 1.11 and/or
wherein the protein fraction has a degree of protein denaturation of at most 5% and/or
a lipid content of more than 5% of the total energy content of the preparation.

In a preferred embodiment of the present invention the packaged, heat-treated beverage preparation has a pH in the range of 3.0-4.7, preferably 3.9-4.6, more preferably 4.0-4.5, the beverage comprising
a total amount of protein of 10 to 45% w/w relative to the weight of the beverage, wherein at least 85% w/w of the protein is BLG preferably at least 90% w/w, and
optionally, sweetener and/or flavour,

wherein:
the turbidity is more than 200 NTU, preferably more than 1000 NTU and/or
the viscosity is at most 200 cP.

In some embodiments of the invention the heat-treated beverage has a shelf-life at 25 degrees C. for at least 6 months, which comprises:
an edible BLG composition as defined in PCT/EP2017/084553 to provide at total amount of BLG of at least 1% (w/w), preferably at least 5% (w/w),
a sweetener, e.g. a sugar sweetener and/or a non-sugar sweetener,
at least one food acid, e.g. citric acid or other suitable food acids,
optionally, a flavouring agent, and
at most 80 mg phosphorus/100 g protein

which has a pH in the range of 2.5-4.0.

In a preferred embodiment of the present invention it relates to use of a protein solution comprising a total amount of protein of 3 to 30% w/w relative to the weight of the solution, wherein at least 85 w/w % of the protein is BLG, preferably at least 90 w/w % for controlling the turbidity of a heat-treated acidic beverage preparation having a pH in the range of 3.0-4.5.

In a preferred embodiment of the present invention it relates to use of a protein solution comprising a total amount of protein of 3 to 30% w/w relative to the weight of the solution, wherein at least 85 w/w % of the protein is BLG, preferably at least 90 w/w % for controlling the astringency of a heat-treated acidic beverage preparation having a pH in the range of 2.0-4.0.

A preferred embodiment of the invention pertains to a heat-treated beverage preparation obtainable by one or more methods described herein.

It should be noted that the embodiments and features described in the context of one of the aspects of the present invention also apply to the other aspects of the invention.

All patent and non-patent references cited in the present application are hereby incorporated by reference in their entirety.

The invention will now be described in further details in the following non-limiting examples.

### EXAMPLES

### Example 1: Methods of Analysis

### Example 1.1: Determination of Protein Nativeness by Intrinsic Tryptophan Fluorescence

Tryptophan (Trp) fluorescence spectroscopy is a well-described tool to monitor protein folding and unfolding. Trp residues buried within native proteins typically display highest fluorescence emission around 330 nm than when present in more solvent exposed positions such as unfolded proteins. In unfolded proteins, the wavelengths for Trp fluorescence emission typically shift to higher wavelengths and are often measured around 350 nm. We here exploit this transition to monitor thermally induced unfolding by calculating the ratio between fluorescence emission at 330 nm and 350 nm to investigate the influence of heating temperature.

The analysis comprises the following steps:
Beverage compositions were diluted to 0.6 mg/ml in MQ water.
300 μl sample was transferred to white 96-well plate avoiding bubbles or 3 mL was transferred to 10 mm quartz cuvette.
The tryptophan fluorescence emission intensity between 310 and 400 nm was recorded from the top by excitation at 295 using 5 nm slits.
Samples were measured using a Cary Eclipse fluorescence spectrophotometer equipped with a plate reader accessory (G9810A) or single cuvette holder.
The emission intensity ratio was calculated by dividing the measured fluorescence emission intensity at 330 nm with the emission intensity at 350 nm, R=I330/I350, and used as a measure of protein nativity.
   R of at least 1.11 describes a predominant native BLG conformation and
   R of less than 1.11 reports on at least partial unfolding and aggregation.

### Example 1.2: Heat-Stability at pH 3.9

Heat-Stability at pH 3.9:

The heat-stability at pH 3.9 is a measure of the ability of protein composition to stay clear upon prolonged pasteurization at pH 3.9.

The heat-stability at pH 3.9 is determined by forming an aqueous solution having a pH of 3.9 and comprising 6.0% w/w protein by mixing a sample of the powder or liquid to be tested with water (or alternatively concentrating it by low temperature evaporation if it is a dilute liquid) and adjusting the pH to 3.9 with the minimum amount of 0.1 M NaOH or 0.1 M HCl required.

The pH-adjusted mixture is allowed to rest for 30 minutes after which 25 mL of the mixture is transferred to a 30 mL thin-walled glass test tube. It is heated to 75.0 degrees C. for 300 seconds by immersion into a water-bath having a temperature of 75.0 degrees C. Immediately after the heating, the glass test tube is cooled to 1-5 degrees C. by transferring it to an ice bath and the turbidity of the heat-treated sample is measured according to Example 1.7.

### Example 1.3: Determination of the Degree of Protein Denaturation of a Whey Protein Composition

Denatured whey protein is known to have a lower solubility at pH 4.6 than at pH values below or above pH 4.6, therefore the degree of denaturation of a whey protein composition is determined by measuring the amount of soluble protein at pH 4.6 relative to the total amount of protein at a pH where the proteins in the solution are stable.

More specifically for whey proteins, the whey protein composition to be analysed (e.g. a powder or an aqueous solution) is converted to:
a first aqueous solution containing 5.0% (w/w) total protein and having a pH of 7.0 or 3.0, and
a second aqueous solution containing 5.0% (w/w) total protein and having a pH of 4.6.

pH adjustments are made using 3% (w/w) NaOH (aq) or 5% (w/w) HCl (aq).

The total protein content (P_{pH 7.0 or 3.0}) of the first aqueous solution is determined according to example 1.5.

The second aqueous solution is stored for 2 h at room temperature and subsequently centrifuged at 3000 g for 5 minutes. A sample of the supernatant is recovered and analysed according to Example 1.5 to give the protein concentration in the supernatant (S_{pH4.6}).

The degree of protein denaturation, D, of the whey protein composition is calculated as:

D=((P_{pH 7.0 or 3.0}−S_{pH 4.6})/P_{pH 7.0 or 3.0})*100%

### Example 1.4 Determination of Protein Denaturation (with pH 4.6 Acid Precipitation) Using Reverse Phase UPLC Analysis

BLG samples (such as non-heated reference and heated BLG beverage compositions) were diluted to 2% in MQ water. 5 mL protein solution, 10 mL Milli-Q, 4 mL 10% acetic acid and 6 mL 1.0M NaOAc are mixed and stirred for 20 minutes to allow precipitation agglomeration of denatured protein around pH 4.6. The solution is filtered through 0.22 μm filter to remove agglomerates and non-native proteins.

All samples were subjected to the same degree of dilution by adding polished water.

For each sample, the same volume was loaded on an UPLC system with a UPLC column (Protein BEH C4; 300 Å; 1.7 μm; 150×2.1 mm) and detected at 214 nm.

The samples were run using the following conditions:

Buffer A: Milli-Q water, 0.1% w/w TFA

Buffer B: HPLC grade acetonitrile, 0.1% w/w TFA

Flow: 0.4 ml/min

Gradient: 0-6.00 minutes 24-45% B; 6.00-6.50 minutes 45-90% B; 6.50-7.00 minutes 90% B; 7.00-7.50 minutes 90-24% B and 7.50-10.00 minutes 24% B.

The area of BLG peaks against a protein standard (Sigma L0130) was used to determine the concentration of native bLG in samples (5 level calibration curve) Samples were diluted further and reinjected if outside linear range.

### Example 1.5: Determination Total Protein

The total protein content (true protein) of a sample is determined by:

1) Determining the total nitrogen of the sample following ISO 8968-1/2|IDF 020-1/2—Milk—Determination of nitrogen content—Part 1/2: Determination of nitrogen content using the Kjeldahl method.

2) Determining the non-protein nitrogen of the sample following ISO 8968-4|IDF 020-4—Milk—Determination of nitrogen content—Part 4: Determination of non-protein-nitrogen content.

3) Calculating the total amount protein as (m_{total nitrogen}−m_{non-protein-nitrogen})*6.38.

### Example 1.6: Determination of Non-Aggregated BLG, ALA, and CMP

The content of non-aggregated alpha-lactalbumin (ALA), beta-lactoglobulin (BLG) and caseinomacropeptide (CMP), respectively was analysed by HPLC analysis at 0.4 mL/min. 25 microL filtered sample is injected onto 2 TSKgel3000PWxl (7.8 mm 30 cm, Tosohass, Japan) columns connected in series with attached pre-column PWxl (6 mm×4 cm, Tosohass, Japan) equilibrated in the eluent (consisting of 465 g Milli-Q water, 417.3 g acetonitrile and 1 mL triflouroacetic acid) and using a UV detector at 210 nm.

Quantitative determination of the contents of native alpha-lactalbumin (Cₐₗₚₕₐ), beta-lactoglobulin (C_{beta}), and caseinomacropeptide (C_{CMP}) was performed by comparing the peak areas obtained for the corresponding standard proteins with those of the samples.

The total amount of additional protein (non-BLG protein) was determined by subtracting the amount of BLG from the amount of total protein (determined according to Example 1.5)

### Example 1.7: Determination of Turbidity

Turbidity is the cloudiness or haziness of a fluid caused by large number of particles that are generally invisible to the naked eye, similar to smoke in air.

Turbidity is measured in nephelometric turbidity units (NTU).

20 mL beverages/samples were added to NTU-glass and placed in the Turbiquant® 3000 IR Turbidimeter. The NTU-value was measured after stabilisation and repeated twice.

### Example 1.8: Determination of Viscosity

The viscosity of beverage preparations was measured using a Rheometer (Anton Paar, Physica MCR301).

3.8 mL sample was added to cup DG26.7. Samples were equilibrated to 22° C., then pre-sheared for 30 sec. at 50 s⁻¹, followed by a 30 sec. equilibrium time and shear rate sweeps between 1 s⁻¹ and 200 s⁻¹ and 1 s⁻¹ were performed.

The viscosity is presented in the unit centipoise (cP) at a shear rate of 100 s⁻¹ unless otherwise stated. The higher the measured cP values, the higher the viscosity.

Alternatively, the viscosity was estimated using a Viscoman by Gilson and reported at a shear rate of about 300 s⁻¹

### Example 1.9: Determination of Colour

The colour was measured using a Chroma Meter (Konica Minolta, CR-400). 15 g sample was added to a small petri dish (55×14.2 mm, VWR Cat #391-0895) avoiding bubble formation. The protein content of the samples was standardised to 6.0 w/w % protein or less.

The Chroma Meter was calibrated to a white calibration plate (No. 19033177). The illuminant was set to D65 and the observer to 2 degree. The color (CIELAB color space, a*-, b*-, L*-value) was measured with lids covering the suspension, as the average of three individual readings in different places of the petri dish.

Demineralised water reference has the following values:

L* 39.97±0.3

a* 0.00±0.06

b* −0.22±0.09 The measurements were converted to delta/difference values based on demineralised water measurement.

delta L*=L_{sample standardised to 6.0 w/w % protein}*−L_{demin. water}*, measured at room temperature.

delta a*=a_{sample standardised to 6.0 w/w % protein}*−a_{demin. water}*, measured at room temperature.

delta b*=b_{sample standardised to 6.0 w/w % protein}*−b_{demin. water}*, measured at room temperature.

The samples is standardized to 6.0 w/w % protein or below.

The L*a*b* colour space (also referred to as the CIELAB space) is one of the uniform colour spaces defined by the International Commission on Illumination (CIE) in 1976 and was used to quantitatively report lightness and hue (ISO 11664-4:2008(E)/CIE S 014-4/E:2007).

In this space, L* indicates lightness (value from 0-100), the darkest black at L*=0, and the brightest white at L*=100.

The colour channels a* and b*, represent true neutral grey values at a*=0 and b*=0. The a* axis represents the green-red component, with green in the negative direction and red in the positive direction. The b* axis represents the blue-yellow component, with blue in the negative direction and yellow in the positive direction.

### Example 1.10 Beverage Stability Test/Insoluble Protein Matter

Whey protein beverage compositions were considered stable if less than 15% of total protein in heated samples precipitated upon centrifugation at 3000 g for 5 minutes:
Approx. 20 g samples were added to centrifuge tubes and centrifugated at 3000 g 5 min.
Kjeldahl analysis of protein before centrifugation and the supernatant after centrifugation were used to quantify protein recovery See example 1.5

The loss of protein is calculated:

$$Denaturation \% = ( \frac{{P}_{total} - {P}_{3000 xg}}{{P}_{total}} ) * 100 \%$$

This parameter is also sometimes referred to as the level of insoluble protein matter and can be used for analyzing both liquid and powder samples. If the sample is a powder, 10 g of the powder is suspended in 90 g demineralized water and allowed to hydrate at 22 degrees C. under gentle stirring for 1 hours. Approx. 20 g of sample (e.g. liquid sample or the suspended powder sample) to centrifuge tubes and centrifugated at 3000 g 5 min. Kjeldahl analysis of protein before centrifugation (Pₜₒₜₐₗ) and the supernatant after centrifugation (P_{3000×g}) were used to quantify protein recovery according to Example 1.5.

The amount of insoluble protein matter is calculated:

$$percentage of insoluble protein matter = ( \frac{{P}_{total} - {P}_{3000 xg}}{{P}_{total}} ) * 100 \%$$

### Example 1.11: Sensory Evaluation

The heat-treated beverage preparations underwent a descriptive sensory evaluation. The beverage preparations had been subjected to heat using plate heat exchangers.

1 volume sample was mixed with 1 volume water and compared to non-heated whey protein isolate, lactic acid and citric acid are also used to form an attribute list prior to the final tasting session:

| Category | Attributes: |
|---|---|
| Aroma | Whey, acidic (sour milk product) |
| Basic taste | Acid, bitter |
| Flavour | Whey, citric acid, lactic acid |
| Mouth feeling | Drying, astringency |

Crackers, white tea, melon and water were used to cleanse the mouth of participants between each sample.

15 mL test sample at ambient temperature (20-25° C.) was served in small cups.

Test samples were each served to 10 individuals three times in three different blocks in randomised order.

The attributes (see table above) were rated on a 15 cm scale with 0=low intensity and 15=high intensity.

The statistical analysis was conducted in ‘Panelcheck’ software using a 3-way ANOVA test for multiple replicates. Samples were fixed and panel was set to random.

Bonferroni correction implying least significance difference values (pairwise comparisons of groups associated to a letter) was used to evaluate significant differences between samples.

### Example 1.12: Determination of Transparency by Imaging

Photographs of beverage preparations were conducted by placing samples in turbidity NTU measuring vials touching a piece of paper with ‘lorem ipsen’ text. Vials were photographed using a smartphone and the inventors evaluated whether the text could be clearly observed through the vial.

### Example 1.13: Determination of Ash Content

The ash content of a food product is determined according to NMKL 173:2005 “Ash, gravimetric determination in foods”.

### Example 1.14: Determination of Conductivity

The “conductivity” (sometimes referred to as the “specific conductance”) of an aqueous solution is a measure of the ability of the solution to conduct electricity. The conductivity may e.g. be determined by measuring the AC resistance of the solution between two electrodes and the result is typically given in the unit milliSiemens per cm (mS/cm). The conductivity may for example be measured according to the EPA (the US Environmental Protection Agency) Method No. 120.1.

Conductivity values mentioned herein have been normalised to 25 degrees C. unless it is specified otherwise.

The conductivity is measured on a Conductivity meter (WTW Cond 3210 with a tetracon 325 electrode).

The system is calibrated as described in the manual before use. The electrode is rinsed thoroughly in the same type of medium as the measurement is conducted on, in order to avoid local dilutions. The electrode is lowered into the medium so that the area where the measurement occurs is completely submerged. The electrode is then agitated so that any air trapped on the electrode is removed. The electrode is then kept still until a stable value can be obtained and recorded from the display.

### Example 1.15: Determination of the Total Solids of a Solution

The total solids of a solution may be determined according NMKL 110 2^{nd} Edition, 2005 (Total solids (Water)—Gravimetric determination in milk and milk products). NMKL is an abbreviation for “Nordisk Metodikkomité for Næringsmidler”.

The water content of the solution can be calculated as 100% minus the relative amount of total solids (% w/w).

### Example 1.16: Determination of pH

All pH values are measured using a pH glass electrode and are normalised to 25 degrees C.

The pH glass electrode (having temperature compensation) is rinsed carefully before and calibrated before use.

When the sample is in liquid form, then pH is measured directly in the liquid solution at 25 degrees C.

When the sample is a powder, 10 gram of a powder is dissolved in 90 ml of demineralised water at room temperature while stirring vigorously. The pH of the solution is then measured at 25 degrees C.

### Example 1.17: Determination of Loose Density and Bulk Density

The density of a dry powder is defined as the relation between weight and volume of the powder which is analysed using a special Stampf volumeter (i.e. a measuring cylinder) under specified conditions. The density is typically expressed in g/ml or kg/L.

In this method, a sample of dried powder is tamped in a measuring cylinder. After a specified number of tappings, the volume of the product is read and the density is calculated.

Three types of densities can be defined by this method:
Poured density, which is the mass divided with the volume of powder after it has been transferred to the specified measuring cylinder.
Loose density, which is the mass divided with the volume of powder after 100 tappings according to the specified conditions in this standard.
Bulk density, which is the mass divided with the volume of powder after 625 tappings according to the specified conditions in this standard.

The method uses a special measuring cylinder, 250 ml, graduated 0-250 ml, weight 190±15 g (J. Engelsmann A. G. 67059 Ludwigshafen/Rh) and a Stampf volumeter, e.g. J. Engelsmann A. G.

The loose density and the bulk density of the dried product are determined by the following procedure.

Pre-Treatment:

The sample to be measured is stored at room temperature.

The sample is then thoroughly mixed by repeatedly rotating and turning the container (avoid crushing particles). The container is not filled more than ⅔.

Procedure:

Weigh 100.0±0.1 gram of powder and transfer it to the measuring cylinder. The volume Vo is read in ml.

If 100 g powder does not fit into the cylinder, the amount should be reduced to 50 or 25 gram.

Fix the measuring cylinder to the Stampf volumeter and let it tap 100 taps. Level the surface with the spatula and read the volume V100 in ml.

Change the number of tabs to 625 (incl. the 100 taps). After tapping, level the surface and read the volume V625 in ml.

Calculation of Densities:

Calculate the loose and the bulk densities expressed in g/ml according to the following formula:

Bulk density=M/V

where M designates weighed sample in grams and V designates volume after 625 tappings in ml.

### Example 1.18: Determination of the Water Content of a Powder

The water content of a food product is determined according to ISO 5537:2004 (Dried milk—Determination of moisture content (Reference method)). NMKL is an abbreviation for “Nordisk Metodikkomité for Næringsmidler”.

### Example 1.19: Determination of the Amounts of Calcium, Magnesium, Sodium, Potassium, Phosphorus (ICP-MS Method)

The total amounts of calcium, magnesium, sodium, potassium, and phosphorus are determined using a procedure in which the samples are first decomposed using microwave digestion, and then the total amount of mineral(s) is determined using an ICP apparatus.

Apparatus:

The microwave is from Anton Paar and the ICP is an Optima 2000DV from PerkinElmer Inc.

Materials:

1 M HNO₃

Yttrium in 2% HNO₃

Suitable standards for calcium, magnesium, sodium, potassium, and phosphorus in 5% HNO₃

Pre-Treatment:

Weigh out a certain amount of powder and transfer the powder to a microwave digestion tube. Add 5 mL 1M HNO₃. Digest the samples in the microwave in accordance with microwave instructions. Place the digested tubes in a fume cupboard, remove the lid and let volatile fumes evaporate.

Measurement Procedure:

Transfer pre-treated sample to DigiTUBE using a known amount of Milli-Q water. Add a solution of yttrium in 2% HNO₃ to the digestion tube (about 0.25 mL per 50 mL diluted sample) and dilute to known volume using Milli-Q water. Analyse the samples on the ICP using the procedure described by the manufacturer.

A blind sample is prepared by diluting a mixture of 10 mL 1M HNO₃ and 0.5 mL solution of yttrium in 2% HNO₃ to a final volume of 100 mL using Milli-Q water.

At least 3 standard samples are prepared having concentrations which bracket the expected sample concentrations.

### Example 1.20: Determination of the Furosine-Value

The furosine value is determined as described in “Maillard Reaction Evaluation by Furosine Determination During Infant Cereal Processing”, Guerra-Hernandez et al, Journal of Cereal Science 29 (1999) 171-176, and the total amount of protein is determined according to Example 1.5. The furosine value is reported in the unit mg furosine per 100 g protein.

### Example 1.21: Determination of the Crystallinity of BLG in a Liquid

The following method is used to determine the crystallinity of BLG in a liquid having a pH in the range of 5-6.

a) Transfer a 10 mL sample of the liquid in question to a Maxi-Spin filter with a 0.45 micron pore size CA membrane.

b) Immediately spin the filter at 1500 g for 5 min. keeping the centrifuge at 2 degrees C.

c) Add 2 mL cold Milli-Q water (2 degrees C.) to the retentate side of the spin filter and immediately, spin the filter at 1500 g for 5 min while keeping the centrifuge cooled at 2 degrees C., collect the permeate (permeate A), measure the volume and determine BLG concentration via HPLC using the method outlined in Example 1.31.

d) Add 4 mL 2M NaCl to the retentate side of the filter, agitate quickly and allow the mixture to stand for 15 minutes at 25 degrees C.

e) Immediately spin the filter at 1500 g for 5 min and collect the permeate (permeate B)

f) Determine the total weight of BLG in permeate A and permeate B using the method outlined in Example 1.31 and convert the results to total weight of BLG instead of weight percent. The weight of BLG in permeate A is referred to as m_{Permeate A} and the weight of BLG in permeate B is referred to as m_{Permeate B}.

g) The crystallinity of the liquid with respect to BLG is determined as:

crystallinity=m_{permeate B}/(m_{Permeate A}+m_{Permeate B})*100%

### Example 1.22: Determination of the Crystallinity of BLG in a Dry Powder

This method is used to determine the crystallinity of BLG in a dry powder.

a) 5.0 gram of the powder sample is mixed with 20.0 gram of cold Milli-Q water (2 degrees C.) and allowed to stand for 5 minute at 2 degrees C.

b) Transfer the sample of the liquid in question to a Maxi-Spin filter with a 0.45 micron CA membrane.

c) Immediately spin the filter at 1500 g for 5 min. keeping the centrifuge at 2 degrees C.

d) Add 2 mL cold Milli-Q water (2 degrees C.) to the retentate side of the spin filter and immediately, spin the filter at 1500 g for 5 min, collect the permeate (permeate A), measure the volume and determine BLG concentration via HPLC using the method outlined in Example 1.31 and convert the results to total weight of BLG instead of weight percent. The weight of BLG in permeate A is referred to as m_{permeate A}

f) The crystallinity of BLG in the powder is then calculated using the following formula:

$$crystallinity = \frac{{m}_{BLG total} - {m}_{permeate A}}{{m}_{BLG total}} * 100 \%$$

where m_{BLG total} is the total amount of BLG in the powder sample of step a).

If the total amount of BLG of powder sample is unknown, this may be determined by suspending another 5 g powder sample (from the same powder source) in 20.0 gram of Milli-Q water, adjusting the pH to 7.0 by addition of aqueous NaOH, allowing the mixture to stand for 1 hour at 25 degrees C. under stirring, and finally determining the total amount of BLG of the powder sample using Example 1.31.

### Example 1.23: Determination of UF Permeate Conductivity

15 mL of sample is transferred to an Amicon Ultra-15 Centrifugal Filter Units with a 3 kDa cut off (3000 NMWL) and centrifugated at 4000 g for 20-30 minutes or until a sufficient volume of UF permeate for measuring conductivity is accumulated in the bottom part of the filter units. The conductivity is measured immediately after centrifugation. The sample handling and centrifugation are performed at the temperature of the source of the sample.

### Example 1.24: Detection of Dried BLG Crystals in a Powder

The presence of dried BLG crystals in a powder can be identified the following way:

A sample of the powder to be analysed is re-suspended and gently mixed in demineralised water having a temperature of 4 degrees C. in a weight ratio of 2 parts water to 1 part powder, and allowed to rehydrate for 1 hour at 4 degrees C.

The rehydrated sample is inspected by microscopy to identify presence of crystals, preferably using plan polarised light to detect birefringence.

Crystal-like matter is separated and subjected to x-ray crystallography in order verify the existence of crystal structure, and preferably also verifying that the crystal lattice (space group and unit cell dimensions) corresponds to those of a BLG crystal.

The chemical composition of the separated crystal-like matter is analysed to verify that its solids primarily consists of BLG.

### Example 1.25: Determination of the Total Amount of Lactose

The total amount of lactose is determined according to ISO 5765-2:2002 (IDF 79-2: 2002) “Dried milk, dried ice-mixes and processed cheese—Determination of lactose content—Part 2: Enzymatic method utilizing the galactose moiety of the lactose”.

### Example 1.26: Determination of the Total Amount of Carbohydrate

The amount of carbohydrate is determined by use of Sigma Aldrich Total Carbohydrate Assay Kit (Cat MAK104-1KT) in which carbohydrates are hydrolysed and converted to furfural and hydroxyfurfurals which are converted to a chromagen that is monitored spectrophotometrically at 490 nm.

### Example 1.27: Determination of the Total Amount of Lipids

The amount of lipid is determined according to ISO 1211:2010 (Determination of Fat Content—Röse-Gottlieb Gravimetric Method).

### Example 1.28: Determination of Brix

Brix measurements were conducted using a PAL-a digital hand-held refractometer (Atago) calibrated against polished water (water filtered by reverse osmosis to obtain a conductivity of at most 0.05 mS/cm).

Approx. 500 μl of sample was transferred to the prism surface of the instrument and the measurement was started. The measured value was read and recorded

### Example 1.29 Determination of Lactoferrin and Lactoperoxidase

The concentration of lactoferrin is determined by an ELISA immunoassay as outlined by Soyeurt 2012 (Soyeurt et al; Mid-infrared prediction of lactoferrin content in bovine milk: potential indicator of mastitis; Animal (2012), 6:11, pp 1830-1838)

The concentration of lactoperoxidase is determined using a commercially available bovine lactoperoxidase kit.

### Example 1.30: Determination the Number of Colony-Forming Units

The determination of the number of colony-forming units per gram sample is performed according to ISO 4833-1:2013(E): Microbiology of food and animal feeding stuffs—horizontal method for the enumeration of microorganisms—Colony-count technique at 30° C.

### Example 1.31: Determination of the Total Amount of BLG, ALA, and CMP

This procedure is a liquid chromatographic (HPLC) method for the quantitative analysis of proteins such as ALA, BLG and CMP and optionally also other protein species in a composition. Contrary to the method of Example 1.6 the present method also measures proteins that are present in aggregated and therefore provides a measure of the total amount of the protein species in the composition in question.

The mode of separation is Size Exclusion Chromatography (SEC) and the method uses 6M Guanidine HCl buffer as both sample solvent and HPLC mobile phase. Mercaptoethanol is used as a reducing agent to reduce the disulphide (S—S) in the proteins or protein aggregates to create unfolded monomeric structures.

The sample preparation is easily achieved by dissolving 10 mg protein equivalent in the mobile phase.

Two TSK-GEL G3000SWXL (7.7 mm×30.0 cm) columns (GPC columns) and a guard column are placed in series to achieve adequate separation of the major proteins in raw materials.

The eluted analytes are detected and quantified by UV detection (280 nm).

Equipment/Materials:
1. HPLC Pump 515 with manual seal wash (Waters)
2. HPLC Pump Controller Module II (Waters)
3. Autosampler 717 (Waters)
4. Dual Absorbance Detector 2487 (Waters)
5. Computer software capable of generating quantitative reports (Empower 3, Waters)
6. Analytical column: Two TSK-GEL G3000SWXL (7.8×300 mm, P/N: 08541). Guard Column: TSK—Guard Column SWxL (6.0×40 mm, P/N: 08543).
7. Ultrasonic Bath (Branson 5200)
8. 25 mm Syringe filter with 0.2 μm Cellulose Acetate membrane. (514-0060, VWR)

Procedure:

Mobile Phase:

A. Stock Buffer Solution.
1. Weigh 56.6 g of Na₂HPO₄, 3.5 g of NaH₂PO₄, and 2.9 g of EDTA in to a 1000 mL beaker. Dissolve in 800 mL of water.
2. Measure pH and adjust to 7.5±0.1, if necessary, with HCl (decrease pH) or NaOH (increase pH).
3. Transfer to a 1000 mL volumetric flask and dilute to volume with water.

B. 6M Guanidine HCl Mobile Phase.

1. Weigh 1146 g of Guanidine HCl in to a 2000 mL beaker, and add 200 mL of the stock buffer solution(A)

2. Dilute this solution to about 1600 mL with water while mixing with a magnetic stir bar (50° C.)

3. Adjust the pH to 7.5±0.1 with NaOH.

4. Transfer into a 2000 mL volumetric flask and dilute to volume with water.

5. Filter using the solvent filtration apparatus with the 0.22 μm membrane filter.

Calibration Standards.

Calibration standards of each protein to be quantified are prepared the following way:
1. Weigh accurately (to 0.01 mg) about 25 mg of the protein reference standard into a 10 mL volumetric flask and dissolve in 10 mL of water.
   This is the protein stock standard solution (51) of the protein
2. Pipette 200 μl of S1 into a 20 ml volumetric flask and dilute to volume with mobile phase.
   This is the low working standard solution WS1.
3. Pipette 500 μL of S1 into a 10 mL volumetric flask and dilute to volume with mobile phase.
   This is standard solution WS2.
4. Pipette 500 μL of S1 into a 5 mL volumetric flask and dilute to volume with mobile phase.
   This is standard solution WS3.
5. Pipette 750 μL of S1 into a 5 mL volumetric flask and dilute to volume with mobile phase.
   This is standard solution WS4.
6. Pipette 1.0 mL of S1 into a 5 mL volumetric flask and dilute to volume with mobile phase.
   This is the high working standard solution WS5.
7. Using graduated disposable pipettes transfer 1.5 mL of WS1-5 into separate vials.
   Add 10 μL of 2-mercaptoethanol to each vial and cap. Vortex the solutions for 10 sec.
   Let the standards stay at ambient temperature for about 1 hr.
8. Filter the standards using 0.22 μm Cellulose Acetate syringe filters.

The purity of protein is measured using Kjeldahl (N×6.38) and the area % from standard solution WS5 using the HPLC.

protein (mg)=“protein standard weight” (mg)×P1×P2

P1=P % (Kjeldahl)

P2=protein area % (HPLC)

Sample Preparation
1. Weigh the equivalent of 25 mg of protein of the original sample into a 25 mL volumetric flask.
2. Add approximately 20 mL of mobile phase and let the sample dissolve for about 30 min.
3. Add mobile phase to volume and add 167 μL of 2-mercaptoethanol to the 25 ml sample solution.
4. Sonicate for about 30 min and afterwards let the sample stay at ambient temperature for about 1½ hours.
5. Mix the solution and filter using 0.22 μl Cellulose Acetate syringe filters.

HPLC System/Columns

Column Equilibration
1. Connect the GPC guard column and the two GPC analytical columns in series.
   New columns are generally shipped in a phosphate-salt buffer.
2. Run water through a new column gradually from 0.1 to 0.5 mL/min in 30 to 60 mins.
   Continue flushing for about 1 hour.
3. Gradually decrease flow rate from 0.5 mL/min to 0.1 mL/min and replace with mobile phase in the reservoir.
4. Increase pump flow rate gradually from 0.1 to 0.5 mL/min in 30 to 60 mins to avoid pressure shock and leave at 0.5 mL/min.
5. Inject ten samples to allow the column to be saturated and wait for the peaks to elute.
   This will aid in the conditioning of the column.
   This step is done without the need of waiting for each injection to be complete before injecting the next.
6. Equilibrate with the mobile phase at least 1 hour.

Calculation of the Results

Quantitative determination of the contents of the proteins to be quantified, e.g. alpha-lactalbumin, beta-lactoglobulin, and caseinomacropeptide, is performed by comparing the peak areas obtained for the corresponding standard proteins with those of the samples. The results are reported as g specific protein/100 g of the original sample or weight percentage of the specific protein relative to the weight of the original sample.

### Example 2: Production of a Spray-Dried, Acidic BLG Isolate Powder

Whey Protein Feed

Lactose-depleted UF retentate derived from sweet whey from a standard cheese production process was filtered through a 1.2 micron filter and had been fat-reduced via a Synder FR membrane prior to being used as feed for the BLG crystallisation process. The chemical composition of the feed can be seen in Table A. We note that all weight percentages of specific proteins, such as BLG, ALA, mentioned in this Example pertain to the weight percentage of the non-aggregated proteins relative to total protein.

Conditioning

The sweet whey feed was conditioned on an ultrafiltration setup at 20 degrees C., using a Koch HFK-328 type membrane (70 m² membrane) with a 46 mill spacer feed pressure 1.5-3.0 bar, to a feed concentration of 21% total solids (TS) ±5, and using as diafiltration medium polished water (water filtered by reverse osmosis to obtain a conductivity of at most 0.05 mS/cm). The pH was then adjusted by adding HCl so that the pH was approx. 5.5. Diafiltration continued until the drop in conductivity of the retentate was below 0.1 mS/cm over a 20 min period. The retentate was then concentrated until the permeate flow was below 1.43 L/h/m². A first sample of concentrated retentate was taken and subjected to centrifugation at 3000 g for 5 minutes. The supernatant of the first sample was used for the determination of BLG yield.

Crystallisation

The concentrated retentate was transferred to a 300 L crystallisation tank where it was seeded with pure BLG crystal material made from rehydrated, spray-dried BLG crystals. Subsequently, the seeded whey protein solution was cooled from 20 degrees C. to approx. 6 degrees C. over approx. 10 hours to allow the BLG crystals to form and grow.

After cooling, a sample of the crystal-containing whey protein solution (the second sample) was taken and the BLG crystals were separated by centrifugation at 3000 g for 5 minutes. The supernatant and crystal pellets from the second sample were subjected to HPLC analysis as described below. The yield of crystallisation was calculated as outlined below and determined to 57%.

TABLE A
Chemical composition of the feed
Feed standardized to 95% total solids

| Protein composition | |
|---|---|
| % w/w of total protein | |
| ALA | 10.2 |
| BLG | 59.6 |
| Other proteins | 30.2 |
| Selected other | |
| components % w/w | |
| Ca | 0.438 |
| K | 0.537 |
| Mg | 0.077 |
| Na | 0.131 |
| P | 0.200 |
| Fat | 0.220 |
| protein | 87 |
| concentration | |

BLG Yield Determination Using HPLC:

The supernatants of the first and second samples were subjected to the same degree of dilution by adding polished water and the diluted supernatants were filtered through a 0.22 μm filter. For each filtered and diluted supernatant the same volume was loaded on an HPLC system with a Phenomenex Jupiter® 5 μm C4 300 Å, LC Column 250×4.6 mm, Ea. and detected at 214 nm.

The samples were run using the following conditions:

Buffer A: MilliQ water, 0.1% w/w TFA

Buffer B: HPLC grade acetonitrile, 0.085% w/w TFA

Flow: 1 mL/min

Column temperature: 40 degrees C.

Gradient: 0-30 minutes 82-55% A and 18-45% B; 30-32 minutes 55-10% A and 45-90% B; 32.5-37.5 minutes 10% A and 90% B; 38-48 minutes 10-82% A and 90-18% B.

Data Treatment:

As both supernatants were treated in the same way, one can directly compare the area of the BLG peaks to calculate a relative yield. As the crystals only contain BLG and the samples all have been treated in the same way, the concentration of alpha-lactalbumin (ALA) and hence the area of ALA should be the same in all of the samples. Therefore, the area of ALA before and after crystallisation is used as a correction factor (cf) when calculating the relative yield.

$${cf}_{α} = \frac{area of {ALA}_{before crystallization}}{area of {ALA}_{after crystallization}}$$

The relative yield is calculated by the following equation:

$${Yield}_{BLG} = ( 1 - \frac{{cf}_{α} \times area of {BLG}_{after crystallization}}{area of {BLG}_{before crystallization}} ) \times 100$$

Acid Dissolution of BLC Crystals

The remainder of the material from the crystallisation tank was separated using a decanter at 350 g, 2750 RPM, 150 RPM Diff. with a 64 spacer and a feed flow of 75 L/h before separation the feed was mixed 1:2 with polished water. The BLG crystal/solid phase from the decanter was then mixed with polished water in order to make it into a thinner slurry before a phosphoric acid was added to lower the pH to approx. 3.0 in order to quickly dissolve the crystals.

After dissolving the BLG crystals, the pure BLG protein liquid was concentrated to 15 Brix on the same UF setup as used to prepare the feed for crystallisation and the pH was adjusted to final pH of approx. 3.8. The liquid BLG isolate was then heated to 75 degrees for 5 minutes and subsequently cooled to 10 degrees C. The heat-treatment was found to reduce the microbial load from 137.000 CFU/g prior to the heat-treatment to <1000 CFU/g after the heat-treatment. The heat-treatment did not cause any protein denaturation and the intrinsic tryptophan fluorescence ratio (I330 nm/I 350 nm) was determined to 1.20 indicating native confirmation of the BLG molecules.

The BLG was dried on a pilot plant spray drier with an inlet temperature of 180 degrees C. and an exit temperature of 75 degrees C. The resulting powder sampled at the exit had a water content of approx. 4% w/w, the chemical composition of the powder is shown in Table. A sample of the dried powder was dissolved and the degree of protein denaturation was determined to 1.5% and the intrinsic tryptophan fluorescence emission ratio (I330/I350) was measured to 1.20.

TABLE B
The composition of the BLG isolate
powder (BDL = below the
detection limit)
BLG isolate powder standardized
to 95% total solids

| Protein composition % | |
|---|---|
| w/w of total protein | |
| ALA | 0.4 |
| BLG | 98.2 |
| Other protein | 1.4 |

Other selected components (% w/w)

| | |
|---|---|
| Ca | BDL |
| K | BDL |
| Mg | BDL |
| Na | BDL |
| P | 0.781 |
| fat | 0.09 |
| protein concentration | 90 |

The bulk density (625 taps) of the spray-dried powder was estimated at 0.2-0.3 g/cm³.

### Example 3: Preparation of Generic Whey Protein Beverage

Dried BLG isolate protein powders containing ≥85% BLG on protein basis are dispersed in about 75% demineralized water required to reach the desired final protein concentration.

Acidic BLG isolate powders is produced as outlined in example 2 while pH 5.5 BLG isolate powder are produced as outlined in example 7 of PCT/EP2017/084553.

As described in PCT/EP2017/084553, dissolution of BLG material may be aided by addition of acid (selected among one or more food-grade acid such as phosphoric acid, hydrochloric acid, citric acid, malic acid or salts in their dissolved or powder forms. If pH is reduced during dissolution by acid addition, the pH should preferably not pass desired target pH (i.e. avoid unnecessary titration with acid and/or base).

Optionally, minerals, sweeteners, flavours, stabilizers, emulsifiers or other components can be added also including sources of fats and carbohydrates.

Adjust to final pH using 10% phosphoric acid (or other food grade acid) or 10% NaOH

Remaining water is added to reach desired protein concentration and the composition is optionally homogenized.

For comparison, whey protein isolate replace the ≥85% BLG product in the making of reference samples while preserving remaining steps.

Samples were stored at 20° C. in a dark environment.

### Example 4: Thermal Treatment of Whey Protein Compositions

Thermal treatment of the beverages was conducted using plate heat exchanger (Manufacturer: OMVE HTST/UHT pilot plant HT320-20) by heating at 120° C. for 20 seconds (High temperature, short time (HTST), results in denaturation of BLG) or 75° C. with 15 seconds to 5 minute holding times (BLG remain native) equipped with a 10 μm bonded Microfibre filter element, Code 12-57-60k (Headline filters). Other heat treatment conditions may also be applied.

Heat-treated beverage composition was tapped at 75-85° C. into 100 mL sterile bottles, then immediately sealed and placed on ice.

In other experiments, the thermal treatment was conducted by transfer of the whey protein source to thin-walled glass vials containing 15-30 mL sample. Vials immersed for 1 to 5 minutes in water baths pre-equilibrated at the target temperature ranging from 75° C. to 95° C. and followed by cooling on ice.

### Example 5: Production of Heat-Treated Beverage Preparation

In the present example BLG beverages and WPI beverages comprising 6% protein and having a pH of 3.7 were prepared.

The BLG beverages were prepared by dissolving a pH 5.5 BLG isolate Powder (as described in example 7 of PCT/EP2017/084553) in demineralized water at 10 degrees C. 10% H₃PO₄ was slowly added to the solution. The final pH was adjusted to pH 3.7.

The solutions were heat-treated at 120° C. for 20 seconds using a plate heat exchanger or heat-treated at 75° C. with 15 seconds to 5 minute holding times as described in example 4. The beverages were tapped to provide a heat sterilized whey protein beverage composition.

WPI beverages were prepared using the same procedure but from a WPI powder.

Below in table 1 is given the composition of the BLG powder used for the preparation of the beverage preparation, for comparison the composition of the WPI is also listed.

TABLE 1
Composition of BLG powder
(pH 5.5 powder) and WPI powder

| Description | Dry B-LG | WPI-B |
|---|---|---|
| ALA (w/w %) | 0.4 | 8 |
| BLG (w/w %) | 95.9 | 57 |
| Ash | 0.76 | 3 |
| Ca | 0.186 | 0.458 |
| Cl | <0.04 | <0.04 |
| Lipid | <0.04 | 0.1 |
| K | 0.0635 | 0.449 |
| Mg | 0.02885 | 0.0818 |
| Na | <0.0250 | 0.324 |
| NO₃ (ppm) | 1.0 | 3.5 |
| NO₂ (ppm) | 0.07 | n.d. |
| NPN | 0.09 | n.d. |
| Phosphorous | <0.025 | 0.215 |
| Protein | 94.57 | 90.45 |

Beverage preparations comprising BLG and WPI having a pH of 3.7 and a protein content of 6% w/w were heat-treated at 120° C. for 20 seconds and 75° C. for 15 seconds, wherein 95.9 w/w % of the proteins was BLG. In the WPI beverage (WPI-B) 57 w/w % of the proteins was BLG. The turbidity (example 1.7), the viscosity (example 1.8) and colour (example 1.9) of the different samples were analysed.

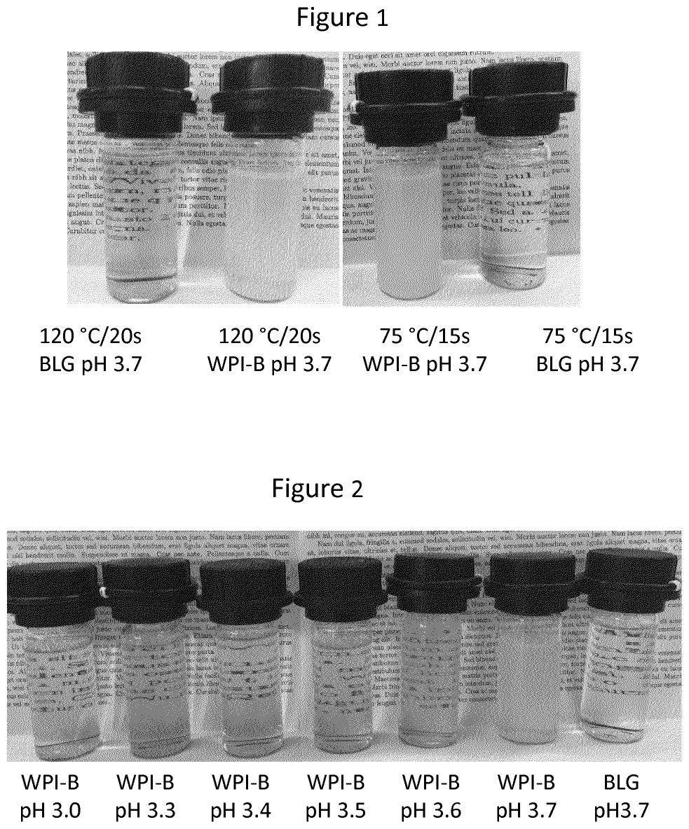

The results are presented in table 2 below and in FIG. 1.

TABLE 2

| | 120° C./20 s | 120° C./20 s | 75° C./15 s | 75° C./15 s |
|---|---|---|---|---|
| | BLG pH 3.7 | WPI-B pH 3.7 | WPI-B pH 3.7 | BLG pH 3.7 |
| Turbidity | 7.0 | 263 | 400 | 1.5 |
| (NTU) | | | | |
| Viscosity | 2.15 | 10.5 | 1.8 | 1.3 |
| (cP) | | | | |

Conclusion:

The turbidity of the BLG samples remained low at 75° C. while the WPI samples had a high turbidity. The WPI samples were also opaque see FIG. 1.

The sterilized BLG samples had a turbidity of 7.0 NTU compared to WPI which had a turbidity of 263 NTU.

The viscosity also remained low.

It is thus possible to produce transparent beverages having a BLG content of about 96 w/w % of the protein content at pH 3.7, while this is not possible in the WPI samples which became opaque under the same conditions.

### Example 6: Demonstrating that the Accessible pH Range for Clear Whey Protein Beverages can be Extended

BLG samples were prepared wherein about 92 w/w % of the 6 w/w % protein was BLG and for comparison two different WPI samples were prepared comprising respectively about 60 w/w % (WPI-A) and 57 w/w % (WPI-B) of BLG.

The 6 w/w % whey protein compositions were prepared as described in example 3 (BLG isolate powders are produced according to example 2) adjusting the final pH using 10% phosphoric acid to obtain selected pH values between 3.0 and 3.9, respectively. In one aspect of the experiment, samples adjusted to pH levels between 3.0 and 3.9 were UHT treated at 120° C. for 20 seconds, tapped, sealed and cooled. In another aspect of the experiment, pH 3.0 and 3.9 samples were pasteurized at 75° C. for 15 seconds as described in example 4.

The turbidity (example 1.7), the viscosity (example 1.8), the colour (example 1.9) and the visual appearance (example 1.12) of the different samples were analyzed.

The results are presented in FIGS. 2-10.

Results:

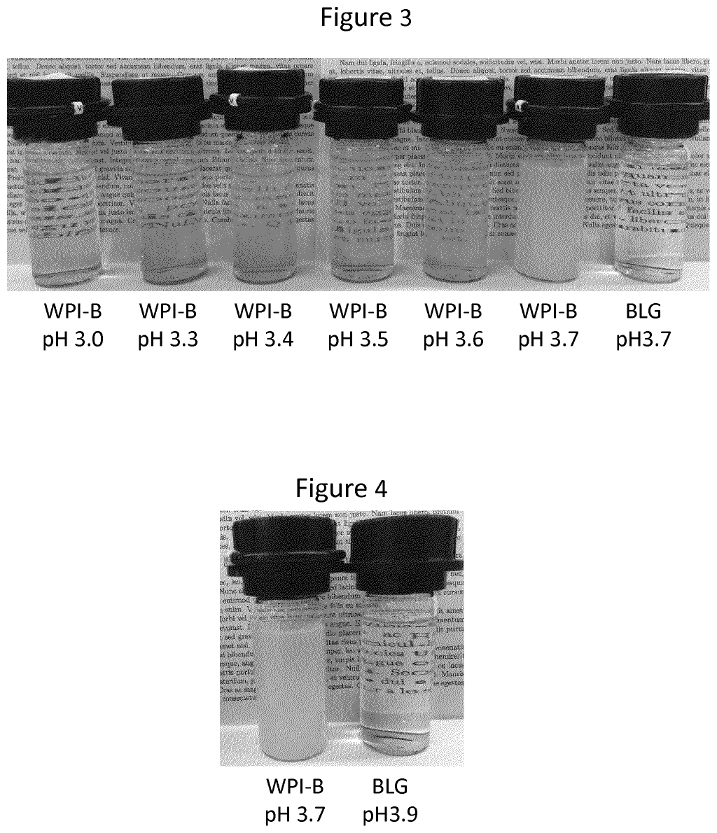

FIG. 2 shows images of WPI-B at pH 3.0-3.7 heat-treated at 120° C. for 20 seconds and BLG beverages at pH 3.7 heat treated at 120° C. for 20 seconds. FIG. 3 shows images of WPI-B at pH 3.0-3.7, heat-treated at 75° C. and BLG at pH 3.7 at heat-treated at 75° C./15 seconds. FIG. 4 shows images of WPI-B at pH 3.7 and BLG beverages at pH 3.9, heated at 75° C. for 15 seconds.

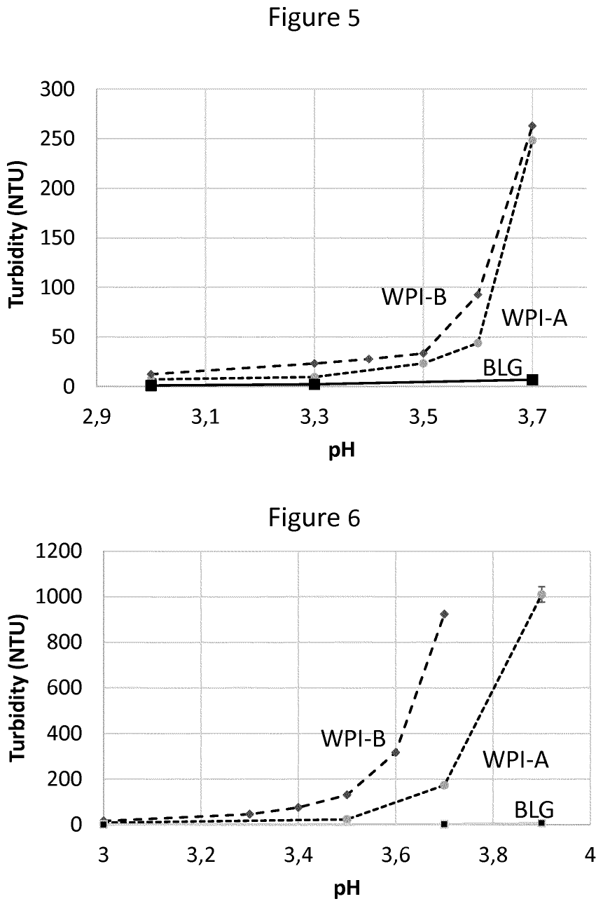

Surprisingly the inventors found that the BLG beverage preparations remain visually clear even at pH 3.7 when it is either UHT sterilized (FIG. 2) and may even exceed pH 3.7 (pH 3.9-4.1) when pasteurized (FIG. 3 and FIG. 4) under which circumstances WPI is opaque. These findings are further supported by turbidity measurements as shown in FIG. 5 (UHT) and FIG. 6 (pasteurization) that remained below 40 NTU even at pH 3.7 and 3.9 where WPI greatly exceed 40 NTU, respectively.

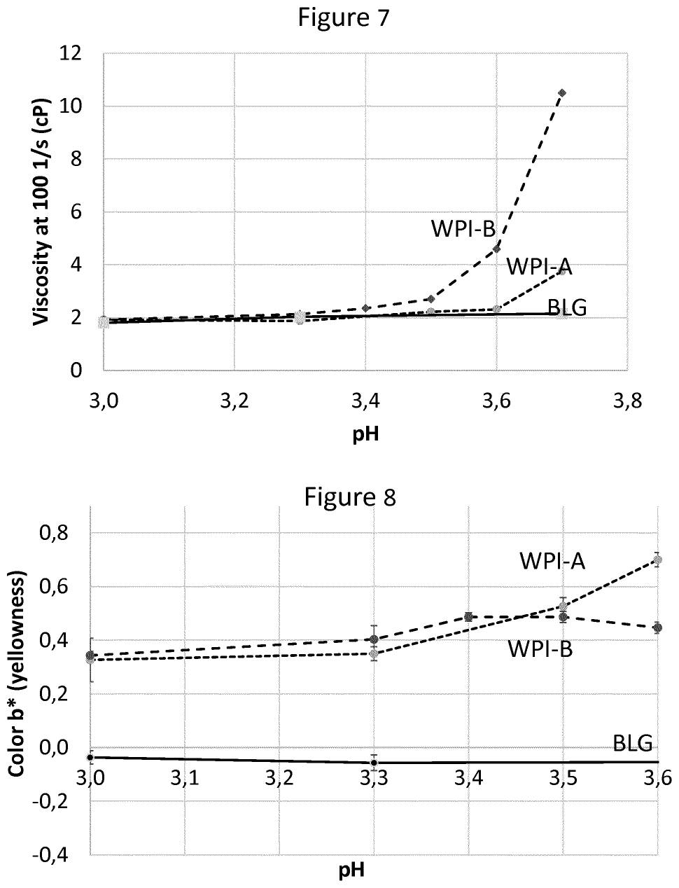

Viscosity remains low upon UHT treatment of BLG beverage preparations. The low viscosities demonstrate that the beverage samples were easily drinkable. The viscosity increases dramatically using WPI especially at high pH values (FIG. 7).

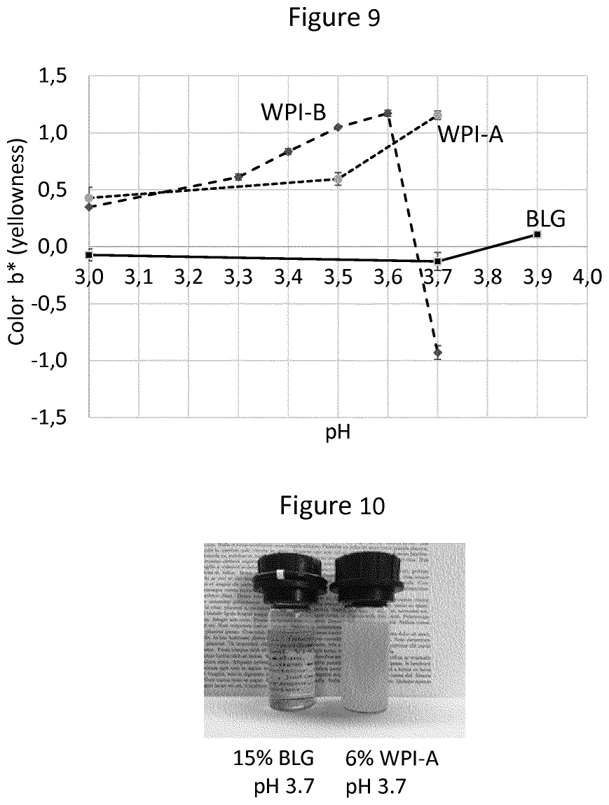

The authors further found that the yellowness (b*-value) of heat-treated WPI beverages comprising a low amount of BLG (both UHT and pasteurization) greatly exceeded BLG up to at least pH 3.7, see FIGS. 8 (UHT) and 9 (pasteurized).

Conclusion:

Use of whey protein beverages wherein at least 85% w/w of the protein is BLG enables at least two significant opportunities to provide whey protein beverages with desired attributes to consumers:
1. Increase pH during thermal treatment providing improvements in visual perception (colour, turbidity), and viscosity when compared to WPI.
2. Allow pasteurization to preserve advantages in 1) while extending accessible pH range even further.

### Example 7: Preparation of Heat Sterilised High Protein Beverage Using BLG

BLG samples were prepared wherein about 92 w/w % of the protein was BLG (0.42 w/w % was ALA), and for comparison WPI samples were prepared using WPI-A wherein about 60 w/w % of the protein was BLG (8 w/w % was ALA), the WPI powder had a pH of 3.3).

A BLG isolate powder product (from example 2, pH of powder was 3.9) was dispersed in tap water to produce beverages having protein concentrations ranging from 6.0 to 30.0 w/w % and adjusted to pH 3.7 using 10% phosphoric acid.

The solutions were thermally treated at 75-120° C. for a duration of time between 15 seconds to 5 minutes as described in Table 3 and immediately cooled on ice.

The viscosity (example 1.8), the nativeness of the proteins determined as intrinsic tryptophan fluorescence emission ratio R=I330/I350 (example 1.1), the visual appearance (example 1.12) and the turbidity (example 1.7) and of the different samples were analysed.

TABLE 3
Analysis data of high protein beverages prepared from BLG at pH 3.7 with
heating at 75° C., 90° C. and 120° C.

| Protein | | Heating | | Fluorescence | | |
|---|---|---|---|---|---|---|
| weight- | | time in | Viscosity | Trp ratio | Visual | Turbidity |
| % | Temperature | seconds | cP | I330/I350 | appearance | NTU |
| 6 | — | — | 1.31 | 1.16 | Transparent | 0.9 |
| 6 | 120° C.. | 20 | 2.15 | 1.08 | Transparent | 6.9 |
| 6 | 750 | 15 | 1.30 | 1.17 | Transparent | 1.0 |
| 10 | 750 | 300 | n.d. | 1.17 | Transparent | — |
| | | | (liquid) | | | |
| 10 | 90° C. | 300 | n.d. | 1.00 | Transparent | — |
| | | | (liquid) | | | |
| 15 | 750 | 15 | 2.91 | 1.19 | Transparent | 2.6 |
| 20 | 750 | 300 | 6.6 ± 0.1 | 1.16 | Transparent | — |
| 25 | 750 | 300 | 10.5 ± 0.1 | 1.16 | Transparent | 9.7 |
| 32 | 750 | 300 | 16.1 ± 0.2 | 1.16 | Opaque | — |

Results:

The results are presented in table 3 above and in FIGS. 10 to 12.

FIG. 10 shows images of 15 w/w % BLG beverage at pH 3.7 heated at 75 C/15 sec that is clear and translucent (left) while a 6% WPI-A at pH 3.7 (right) heated at 75 C/15 sec was opaque.

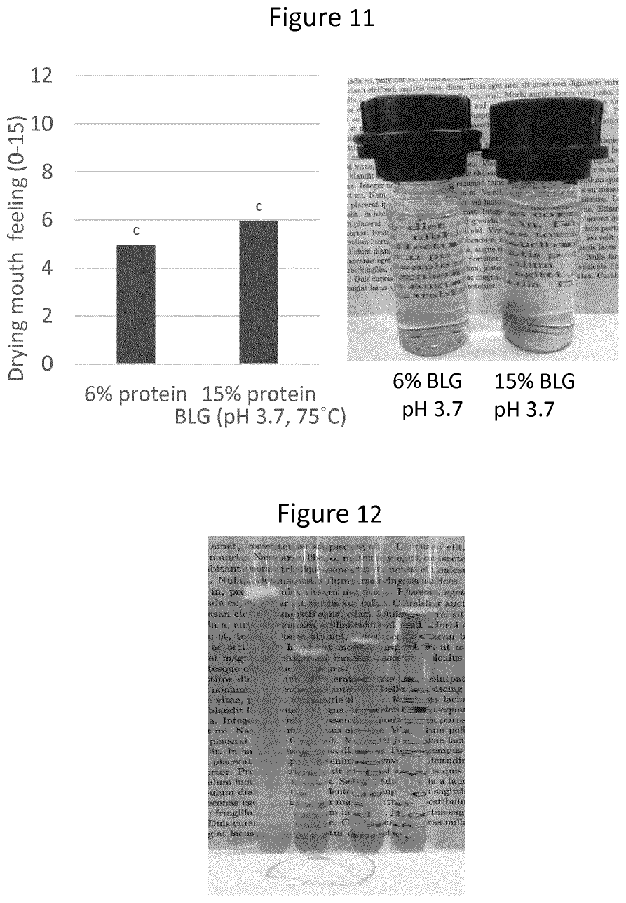

FIG. 11 shows sensory evaluation of high protein BLG beverage compositions and images of 6 w/w % and 15 w/w % BLG samples at pH3.7, both samples are clear.

FIG. 12 shows high protein beverage preparations prepared by heating of BLG beverages having a protein content of 30 w/w %, 27.5 w/w %, 25 w/w %, 20 w/w % (left to right) at 75° C. for 5 minutes all samples had a low viscosity and were liquid.

The inventors surprisingly found that all solutions remained at low viscosity even when heated at 75° C. for up to 5 minutes suggesting little or no denaturation.

Viscosities observed at high protein typical of non-aggregated, native proteins (flow behavior described by (Inthavong, Kharlamova, Nicolai, Chassenieux, & Nicolai, 2016) stating around 10 cP at 200 g/l.

Tryptophan fluorescence spectroscopy confirmed that BLG remains in native conformation as evidenced having an intrinsic tryptophan emission ratio (I330/I350) of at least 1.11 when heated gently (75° C.) whereas more severe heating caused denaturation as shown by intrinsic tryptophan emission ratio (I330/I350) of less than 1.11.

RP-HPLC analysis confirmed the tryptophan fluorescence results revealing 3.6 denaturation of a 6% BLG beverage heated at 75° C. for 5 min and 41% denaturation when heated at 95° C. for 5 minutes.

It was shown that viscosity remained low even after heating.

It was found that BLG beverage preparations can be heated above the denaturation temperature. Heating at 95° C./5 min did, however, result in gelation for BLG beverages comprising above 16 w/w % protein whereas 10% at 90° C./5 min and 6% at 120° C./15 sec remained liquid. As evidenced by a lowering of the intrinsic tryptophan emission ratio (I330/I350), at least partial denaturation/aggregation occurs under these heating conditions.

To the inventors big surprise, the sensory panel (for analysis see example 1.11 and FIG. 11) did not identify significant differences in drying mouthfeel of 6 and 15% BLG beverage preparations heated at 75° C., clearly suggesting a use of high protein beverages for e.g. consumers having difficulties in swallowing.

### Example 8: Whey Protein Beverage Preparations with Improved Taste

BLG samples and WPI samples were prepared. The composition of the samples is shown below.

The used BLG isolate powder is produced according to example 2.

| | BLG | WPI -A |
|---|---|---|
| w/w % BLG | 92 | 60 |
| of protein | | |
| w/w % ALA | 0.42 | 8 |
| of protein | | |
| pH of powder | 3.9 | 3.0 |

The samples were analysed by a sensory panel of 10 people (see example 1.11). The WPI samples were more yellow and had a higher b*-value and they had a higher turbidity than the BLG beverages, especially at higher pH values. The analysis data is presented in table 3.

TABLE 4
Analysis data of whey protein beverages prepared from BLG at pH 3.0 and pH 3.7 with
heating at 75° C. and 120° C.

| | WPI-A | WPI-A | BLG | BLG | BLG | BLG | BLG |
|---|---|---|---|---|---|---|---|
| | (6 %) | (6 %) | (6 %) | (6 %) | (6 %) | (6 %) | (15 %) |
| | pH 3.0 | pH 3.0 | pH 3.0 | pH 3.7 | pH 3.0 | pH 3.7 | pH 3.7 |
| | 120° C./20 s | 75° C./15 s | 120° C./20 s | 120° C./20 s | 75° C./15s | 75° C./15s | 75° C./15 s |
| NTU | 7.17 | 8.63 | 1.02 | 7.0 | 0.88 | 0.99 | 2.6 |
| cP | 2.19 | 1.70 | 1.75 | 2.15 | 1.49 | 1.38 | 2.91 |
| b* | 0.36 ± 0.03 | 0.30 ± 0.03 | −0.01 ± 0.06 | −0.05 ± 0.01 | −0.07 ± 0.05 | −0.07 ± 0.02 | 0.15 ± 0.02 |
| L* | 39.7 ± 0.26 | 39.7 ± 0.2 | 39.7 ± 0.2 | 39.9 ± 0.05 | 39.8 ± 0.12 | 38.9 ± 0.24 | 39.9 ± 0.12 |
| a* | −0.14 ± 0.03 | 0.02 ± 0.05 | −0.01 ± 0.03 | −0.05 ± 0.04 | −0.05 ± 0.01 | −0.08 ± 0.03 | −0.1 ± 0.05 |

Turbididy (NTU), viscosity at 100 s⁻¹ (cP) and colour values b*, L* and a*.

Visual appearance of the samples in table 4 is shown in FIG. 13.

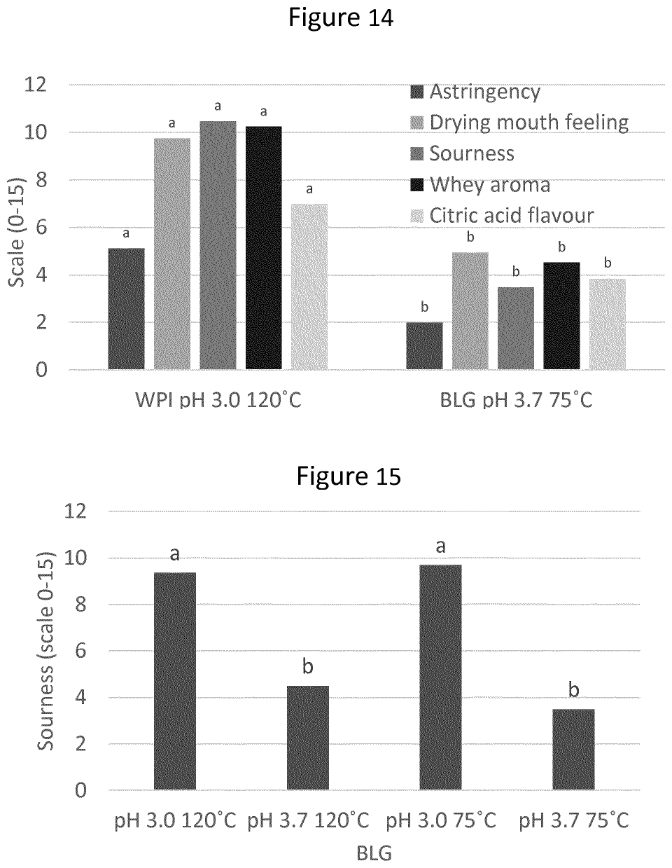

The data from the sensory evaluation is shown in FIGS. 14-18.

For calculation of Delta b* the following formula is used:

delta b*=b_{sample standardized to 6.0 w/w % protein}*−b_{demin. water}*, measured at room temperature.

For calculation of Delta a* the following formula is used:

delta a*=a_{sample standardized to 6.0 w/w % protein}*−a_{demin. water}*, measured at room temperature.

For calculation of Delta L* the following formula is used:

delta L*=L_{sample standardized to 6.0 w/w % protein}*−L_{demin. water}*, measured at room temperature.

The colour values for demineralized water are:

L*=39,97, a*=0 and b*=−0.22.

Results:

By exploiting the opportunity of increasing pH and decreasing the heating temperature while maintaining clarity and colourless characteristic, a significant different in the taste between the beverages produced with WPI-A and BLG was observed. The BLG beverage has a lower astringency, drying mouthfeeling, sourness, whey aroma and citric acid flavour compared to the WPI-beverage, shown in FIG. 14.

FIG. 15 shows that by increasing the pH to 3.7 before heat-treatment the acid taste in BLG beverages is decreased both at 120° C. and 75° C. while retaining product clarity and low colour. This was not possible with WPI, because no transparent and clear beverage can be produced at pH 3.7, as seen in table 2 and in FIG. 1.

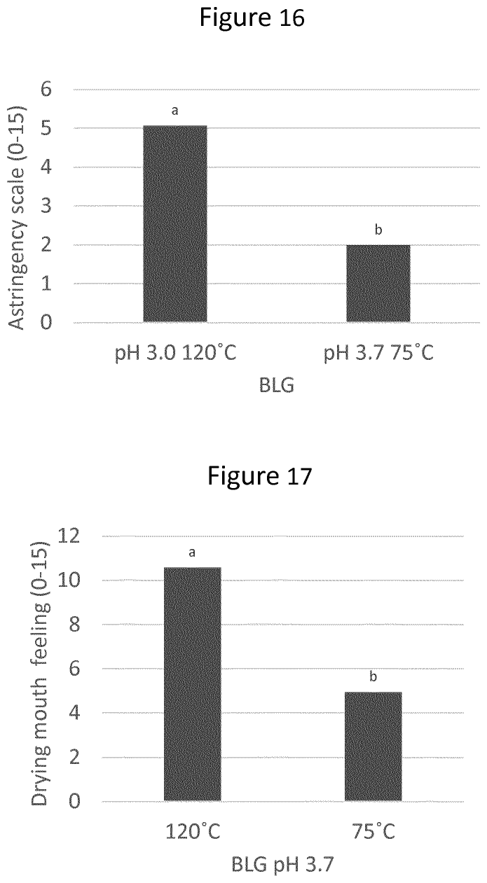

FIG. 16 demonstrates a significant reduction in astringency when both temperature and pH are altered from pH 3.0, 120° C./20 sec to pH 3.7, 75° C./15 sec.

FIG. 17 demonstrates a significant decrease in drying mouthfeel by lowering the heating temperature from 120° C./20 sec to 75° C./15 sec (Native at 75° C. versus denaturized proteins at 120° C.).

FIG. 18 demonstrates that whey aroma is reduced when maintaining BLG in native state by using 75 C/15 sec heating at pH 3.7 where transparent and clear, colourless WPI beverages cannot be produced.

It was not possible to produce a clear beverage with WPI at pH 3.7 and heat-treated at 75° C./15 s see also FIG. 3.

### Example 9: Low Colour Sweetened BLG Beverage Preparations

6% w/w BLG beverages were prepared, see composition of the BLG powder below. The beverages were prepared as described in example 3.

| | BLG |
|---|---|
| w/w % BLG | 92 |
| of protein | |
| w/w % ALA | 0.42 |
| of protein | |
| pH of powder | 3.9 |

The prepared BLG beverages comprised 6% protein and had a pH of 3.7 and pH 4.3.

8 w/w % sucrose was used as the carbohydrate sucrose. Tests were also performed with the high-intensity sweetener sucralose. The samples were subjected to a heat-treatment of 93′C for 4 min in a water bath, then cooled in an ice bath.

The clarity (example 1.12), colour (example 1.9), turbidity (example 1.7) and viscosity (example 1.8) of the different samples were analyzed.

The results are presented in table 4 below.

TABLE 5
Addition of sucrose to 6% protein BLG samples.

| pH 3.7 | Turbidity (NTU) | Viscosity (cP)* | Colour |
|---|---|---|---|
| 0% sucrose | 6.71 | 0.939 | L* 39.89 ± 0.02 |
| | | | a* −0.07 ± 0.01 |
| | | | b* 0.01 ± 0.00 |
| 8% sucrose | 5.91 | 1.52 | L* 39.90 ± 0.08 |
| | | | a* −0.07 ± 0.03 |
| | | | b* 0.05 ± 0.04 |

*Viscoman was used.

Results:

It was found that sweetened BLG beverages can be produced using 8% sucrose as sweetener and subjecting them to a heat-treatment of 93′C for 4 min. The addition of sucrose had only a weak impact on the viscosity, turbidity and clarity, (see table 5) also the colour was not affected by the addition of sucrose.

A BLG beverage with additives typically present in commercial beverages for e.g. sports nutrition, a 6% w/w protein BLG beverage at pH 3.7° C., heat-treated for 75° C., 5 min, was prepared. See table 5 below.

TABLE 6
Example of a commercial product.

| Ingredients | Amount | Unit |
|---|---|---|
| BLG | 660 | g |
| Trisodium-citrate | 1.0 | g |
| Sucralose 100% | 1.17 | g |
| 10% phosphoric acid | 47 | G |
| Add Water to 10 kg | 9.3 | kg |

TABLE 7
Results of the two recipes.

| | BLG without additives | BLG with additives |
|---|---|---|
| NTU | 1.74 | 1.63 |
| cP | 1.28 | 1.27 |
| b* | −0.07 ± 0.06 | −0.11 ± 0.01 |
| L* | 38.73 ± 0.24 | 39.78 ± 0.13 |
| a* | 0.01 ± 0.04 | 0.01 ± 0.03 |

Results:

It can be seen in table 7 that both the BLG beverages with additives and the BLG beverages without additives remain at low viscosity, transparent and essentially colourless.

### Example 10: Exemplary Process for Clean BLG Beverage Preparations Comprising Added Minerals

The BLG powder used in this example had a pH of 5.5, comprising about 96% w/w of the protein as BLG (and 0.4% w/w of the protein as ALA).

The acidic BLG isolate powder was prepared according to example 2, and the beverage preparations were prepared according to example 5.

High Temperature Heat-Treatment of Beverage Preparations:

6% BLG beverage preparations having a pH of 3.7, were prepared. KCl and CaCl₂ was added in liquid form from 1M stock solutions. They were heat-treated at <95° C. for 5 min.

Results:

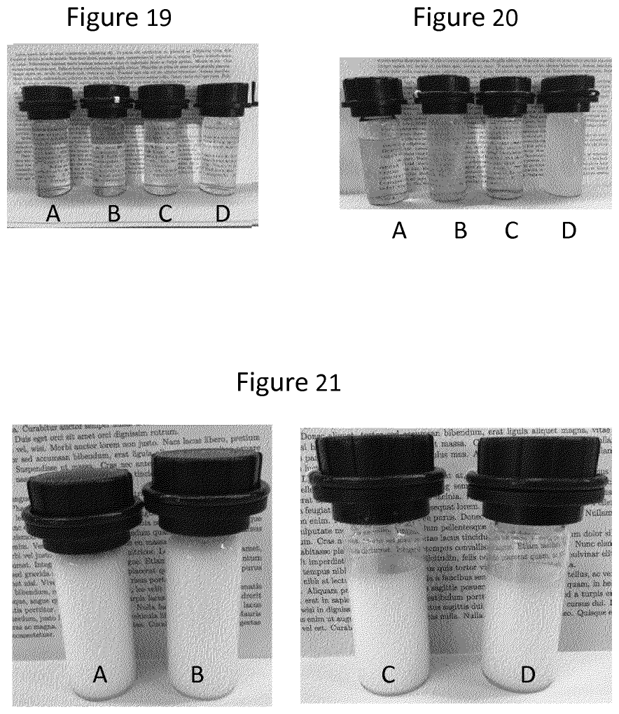

The results are summarized in table 8 below and in FIG. 19.

FIG. 19 shows images of 6% BLG beverages heat-treated at 95° C. for 5 min, pH 3.7 and minerals added.

A: 0 mM added mineral

B: 15 mM added CaCl₂)

C: 20 mM added KCl

D: 10 mM added KCl and 15 mM CaCl₂).

Turbidity of BLG beverage preparations with added minerals (0-20 mM KCl, 0-15 mM CaCl₂) or 10 mM CaCl₂) and 10 mM) remained below 30 NTU when heated at 95° C. for 5 min at pH 3.7.

Gelation was observed at 30 mM added KCl (turbid gel).

Gelation was observed at 20 mM added CaCl₂) (clear gel).

The results clearly suggest that protein composition matters more than mineral difference to WPI, because the amount of added minerals in table 8 greatly exceed the difference between BLG and WPI product(s).

Samples remain clear (see FIG. 19) and had a low viscosity within limits in table 8 below:

TABLE 8
Viscosity and Turbidity of BLG beverages
after addition of minerals (CaCl₂ and KCl),
heated at 95° C. for 5 min, pH 3.7.

| Added | Added | Turbidity | *Viscosity, |
|---|---|---|---|
| CaCl₂, mM | KCl, mM | NTU | cP |
| 0 | 0 | 13.8 | 0.77 ± 0.1 |
| 15 | 0 | 25.7 | 1.37 ± 0.2 |
| 0 | 20 | 19.6 | 1.08 ± 0.1 |
| 10 | 20 | 23.9 | 1.44 ± 0.3 |

*Viscoman was used.

Low Temperature Heat-Treatment of Beverage Preparations

6% BLG beverage preparations having a pH of 3.7 were prepared. KCl and CaCl₂) was added in liquid form from 1M stock solutions. They were heat-treated at pasteurization temperatures of 75° C. for 5 min.

Results.

The inventors surprisingly found that exceptionally high mineral concentrations are allowed when using pasteurization temperatures (75° C., 5 min). See table 9 below.

FIG. 20 shows images of 6% BLG beverages pH 3.7, heat-treated at 75° C. for 5 min. and minerals added.

A: 0 mM added mineral,

B: 100 mM added KCl,

C: 100 mM added CaCl₂),

D: 100 mM added KCl and 100 mM added CaCl₂)

The beverage preparations remained clear even when 100 mM KCl or 100 mM CaCl₂) were added to the beverage composition prior to heating, see FIG. 20. Further, the viscosity was surprisingly low even when both 100 mM KCl and 100 mM CaCl₂) were added.

TABLE 9
Viscosity and Turbidity of BLG beverages after addition of
minerals (CaCl₂ and KCl), heated at 75° C. for 5 min, pH 3.7.

| | | | | | *Viscosity |
|---|---|---|---|---|---|
| | | | | Turbidity | cP |
| Added minerals | pH | Protein | Heating | NTU | avg ± std dev |
| 0 | 3.7 | 6% | 75° C., 5 min | 5.4 | 0.8 ± 0.1 |
| 30 mM KCl | 3.7 | 6% | 75° C., 5 min | 6.9 | 0.7 ± 0.1 |
| 40 mM CaCl2 | 3.7 | 6% | 75° C., 5 min | 6.6 | 0.9 ± 0.1 |
| 100 mM KCl | 3.7 | 6% | 75° C., 5 min | 15 | 0.9 ± 0.2 |
| 100 mM CaCl2 | 3.7 | 6% | 75° C., 5 min | 68 | 0.9 ± 0.1 |
| 100 mM KCl + | 3.7 | 6% | 75° C., 5 min | 325 | 0.9 ± 0.1 |
| 100 mM CaCl2 | | | | | |

*Viscoman was used.

### Example 11: Milky Whey Protein Beverages, High Temperature Heat-Treatment

An exemplary process for producing an opaque and milky beverage comprising BLG and optionally a source of carbohydrate. BLG powder is dissolved in tap water and adjusted to pH according to Example 3 and thermally treated at 93° C. for 4 minutes. The BLG beverages comprised about 92% w/w of the protein as BLG and about 0.42% w/w of the protein as ALA, the beverages are produced based on a acidic BLG isolate powder having a pH of 3.9 (example 2).

6% BLG beverages were prepared having a pH of 4.3. 8% sucrose was added as carbohydrate source. Turbidity, viscosity, colour and transparency were measured according to the procedures described in examples 1.7, 1.8, 1.9 as well as the beverage stability as in example 1.10.

The results are presented in tables 10 and 11 below and in FIG. 21.

TABLE 10
Stability of milky beverages comprising BLG,
heat-treatment of 93° C./4 min. 6% protein and pH 4.3

| | | Turbidity | *Viscosity | |
|---|---|---|---|---|
| 0% sucrose | Brix % | (NTU) | (cP) | Colour |
| Before | 7.2 | >10000 | 1.15 | L* 85.15 ± 0.05 |
| centrifugation | | | | a* −1.24 ± 0.01 |
| | | | | b* −1.95 ± 0.00 |
| After 3000 g | 6.6 | >10000 | 0.87 | L* 79.21 ± 0.20 |
| 5 min. | | | | a* −1.72 ± 0.01 |
| | | | | b* −4.12 ± 0.01 |

*Viscoman was used.

TABLE 11
Stability of a milky BLG beverage also comprising sucrose,
heat-treatment of 93° C./4 min. 6% protein and pH 4.3

| | | Turbidity | Viscosity | |
|---|---|---|---|---|
| 8% sucrose | Brix % | (NTU) | (cP) | Colour |
| Before | 14.2 | >10000 | 1.4 | L* 81.68 ± 0.19 |
| centrifugation | | | | a* 4.51 ± 0.03 |
| | | | | b* −3.01 ± 0.01 |
| After 3000 g | 14.4 | >10000 | 1.33 | L* 76.55 ± 0.20 |
| 5 min. | | | | a* −1.88 ± 0.02 |
| | | | | b* −4.87 ± 0.01 |

WPI samples were prepared comprising 6% protein and having a pH of 4.3 The WPI samples were thermally treated at 94° C. for 5 minutes. 0% sucrose or 8% sucrose was added to the WPI-A sample, while 0% sucrose or 6% sucrose was added to the WPI-B sample.

| | BLG | WPI -A | WPI-B |
|---|---|---|---|
| w/w % BLG | 92 | 60 | 8 |
| of protein | | | |
| w/w % ALA | 0.42 | 57 | 10 |
| of protein | | | |
| pH of powder | 3.9 | 3.0 | 6.8 |

Results:

FIG. 21 illustrates stability of milky BLG beverages, pH 4.3, with and without sucrose, heat-treated at 93° C. for 4 minutes. A: 0% sucrose (before centrifugation), B: 8% sucrose (before centrifugation), C: 0% sucrose (after centrifugation), D: 8% sucrose (after centrifugation)

The results presented in tables 10 and 11 and FIG. 21 demonstrate that high end pH such as pH 4.3 enable manufacture of milky beverages, which is preferred in some embodiments of the invention, for instance when the consumer prefers a whey protein beverage with a milky appearance. It was also found that even at a pH of 4.3 the viscosity was low, both for preparations with and without sucrose.

The colour also remained neutral. This is in particular preferred by the consumers, who prefer that a milky beverage does not have a yellowish colour. A yellowish colour is seen when the b*value is high.

It was also found that the beverages were stable as evidenced by <15% decrease in protein and high turbidity also after centrifugation at 3000×g for 5 minutes.

It was not possible to produce milky 6 w/w % protein WPI beverages based on WPI-A or WPI-B having a pH of 4.3 as they gelled and thus had a high viscosity, this applied for both WPI samples both with and without added sucrose.

### Example 12: Milky Whey Protein Beverages, Low Temperature Heat-Treatment for Prolonged Time

An exemplary process for producing a milky beverage comprising BLG at different pH. BLG powder is dissolved in tap water and adjusted to pH 4.2-4.5 using 10% phosphoric acid according to Example 3. The preparations were thermally treated at 75° C. for 5 minutes and had a protein content of 6% w/w. The BLG beverages comprised about 92% w/w of the protein as BLG and 0.42% w/w of the protein as ALA and are produced based on a BLG powder having a pH of 3.9.

Turbidity, viscosity, colour and visual clarity were measured according to the procedures described in examples 1.7, 1.8, 1.9 and 1.12.

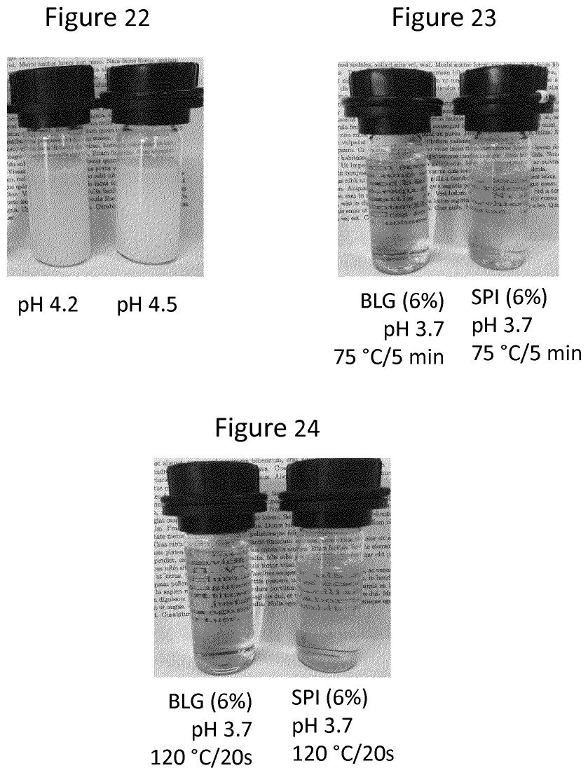

The results are presented in table 12 below and in FIG. 22.

FIG. 22 shows images of opaque 6% protein BLG beverages prepared by heating at 75° C. for 5 min at pH 4.2-4.5.

TABLE 12
Properties of opaque BLG beverages at pH
4.2-4.5 after heating at 75° C. for 5 minutes.

| | pH 4.2 | pH 4.5 |
|---|---|---|
| Turbidity | 2489.6 | 4282.9 |
| (NTU) | | |
| Viscosity (cP) | 0.954 | 0.943 |
| L* | 32.33 ± 0.02 | 40.14 ± 0.06 |
| a* | −0.23 ± 0.05 | −0.67 ± 0.02 |
| b* | −1.34 ± 0.01 | −3.42 ± 0.01 |

Results:

It was found that the beverages at pH 4.2 to 4.5 had a milky and opaque appearance and a high turbidity, while still having a low viscosity.

### Example 13: Colourless Whey Protein Beverage Containing >85% bLG

Beverage preparations were prepared wherein about 92% w/w of the protein is BLG and about 0.42% w/w of the protein is ALA (pH of the BLG-isolate powder was 3.9), see example 3.

For comparison whey protein samples comprising SPI (serum protein isolate) comprising about 80% w/w BLG and about 4% w/w ALA were prepared (pH of the SPI-powder was 6.7).

The samples had a protein content of 6% w/w.

pH of the beverages were adjusted to pH 3.7.

Turbidity, viscosity, colour and transparency of the preparations were measured according to the procedures described in examples 1.7, 1.8, 1.9 as well as beverage stability as in example 1.10.

The results are presented in table 13 below and in FIGS. 23 and 24.

TABLE 13
Properties of BLG and SPI beverages subjected to different
heat-treatments.

| | BLG, without | SPI, without | SPI 75° C. | SPI 95° C. |
|---|---|---|---|---|
| pH 3.7 | heat treament | heat-treatment | 5 min. | 5 min. |
| Turbidity | 1.47 | 21.82 | 52.64 | 74.21 |
| NTU | | | | |
| Viscosity | 1.31 | 0.783 | 1.14 | 1.51 |
| (cP) | | 0.744 | 1.00 | 1.36 |
| L* | 39.86 ± 0.03 | 39.41 ± 0.05 | 39.36 ± 0.07 | 39.36 ± 0.08 |
| a* | −0.03 ± 0.03 | −0.30 ± 0.01 | −0.29 ± 0.02 | −0.28 ± 0.02 |
| b* | −0.08 ± 0.04 | 1.53 ± 0.01 | 1.43 ± 0.02 | 1.52 ± 0.01 |

The viscosity was measured on Viscomann (example 1.8).

Results:

It was found that the viscosity of SPI (about 80% BLG, about 4% ALA) increased more due to heat-treatment compared to the BLG preparations at pH 3.7.

Further the SPI beverages had higher b*values and therefore a more yellowish colour than the BLG samples.

### Example 14: Nutritional Whey Protein Beverage Comprising ≥85% BLG, a Source of Carbohydrate and a Source of Fat

Example 14 describes an exemplary process for preparing a heat sterilized beverage preparation wherein at least 85% w/w of the protein is BLG.

The inventors have surprisingly found that the BLG beverages (≥85%) accept surprisingly large mineral concentrations to be present during sterilization by pasteurization at 75° C. with holding times for up to at least 5 minutes (Example 10) since a 6% nutritional composition with 100 mM added KCl and 100 mM added CaCl₂) remained liquid (viscosity at about 1 cP) even after heating at 75° C. for 5 minutes.

Since heat stability of whey proteins often suffer at high mineral dosages, we therefore investigated further the opportunity to produce nutritionally complete acid BLG beverages to produce sterilized nutritional beverages comprising ≥85% BLG, a source of carbohydrate, a source of fat and minerals in a combination that meet current FSMP (Foods for Special Medical Purposes) requirements.

Dissolving protein and mixing with lipids and carbohydrates in example ratios based on the energy distribution as described in Table 14.

Food grade acid and minerals were selected to accommodate requirements set for food for special medical purposes (FSMP).

Vitamins may further be supplied in the beverage to meet FSMP requirements and produce nutritionally complete nutritional supplements.

TABLE 14
Composition of exemplary nutritional composition containing
sources of protein, carbohydrate and fat.

| | | | | Energy |
|---|---|---|---|---|
| | | Concentration, | Energy, | distribution |
| Component | Source | % | kJ/100 mL | E % |
| Protein | BLG | 6 | 100.8 | 20% |
| Carbohydrate | Sucrose | 13.5 | 226.8 | 45% |
| Fat | Rapeseed oil | 4.7 | 176.4 | 35% |
| Sum | | 24.2 | 504 | |

A 6 w/w % BLG nutritional beverage also comprising 13.5 w/w % sucrose and 4.7 w/w % rapeseed oil was mixed at 70° C. Composition of protein, fat and carbohydrate selected to accommodate recommendations for medical nutrition.

In certain aspects, (1) 40 mM KCl and 14 mM CaCl₂) or (2) 80 mM KCl and 28 mM CaCl₂) was added together with additional components or (3) without further mineral additions as indicated in table 14.

The solutions were homogenized at 200 bar.

The solution was thermally treated by immersion in water bath at 75° C. or 95° C. for 5 minutes and cooled on ice.

TABLE 15
Nutritional compositions containing BLG, a source of
carbohydrate, fats and added minerals.

| | | Turbidity | | Viscosity |
|---|---|---|---|---|
| Treatment | Minerals | NTU | Trp ratio | (cP or mPas) |
| None | As is | 3607 | 1.18 | 2.93 |
| 75 C./5 min | As is | 3349 | 1.18 | 3.20 |
| 75 C./5 min | 40 mM KCl | 3373 | 1.18 | 3.25 |
| (1) | 14 mM CaCl2 | | | |
| 75 C./5 min | 80 mM KCl | 3274 | 1.17 | 2.96 |
| (2) | 24 mM CaCl2 | | | |

Results:

It was found that opaque beverages can be produced using BLG in combination with sources of fat and carbohydrates by heating at 75° C. and 95° C.

At 75° C. it remains in native state (it had a Trp flu ratio of 1.18 despite that it comprised fat), while it causes denaturation (Trp flu) at 95 degrees C. The viscosity remains low. As it was possibly to maintain the native conformation it enables administration of minerals which are critical for medical nutrition (FSMP requirements). Further the ability of the nutritional compositions to remain liquid in the presence of selected minerals clearly suggests the feasibility for use within medical nutrition.

### Example 15: Low Phosphorus Protein Beverage

Four low phosphorus beverage samples are prepared using the purified BLG product from Example 3 (the crystal preparation obtained from feed 3). All the dry ingredients are mixed with demineralised water to obtain 10 kg of each sample and allowed to hydrate for 1 hour at 10 degrees C.

| | Beverage sample | | | |
|---|---|---|---|---|
| Ingredient (% w/w) | A | B | C | D |
| Dried, purified BLG | 5.0 | 10.0 | 5.0 | 10.0 |
| from Ex. 3, feed 3 of | | | | |
| PCT/EP2017/084553 | | | | |
| Citric acid | To pH 3.5 | To pH 3.5 | To pH 3.0 | To pH 3.0 |
| Sucrose | 10 | 10 | 10 | 10 |
| Demineralised water | To 100% | To 100% | To 100% | To 100% |

The samples are subjected to 90 degrees C. for 180 seconds and filled aseptically in sterile containers.

The packaged beverages have a shelf-life of at least 1 year at ambient temperature.

All ingredients used for preparing the 5 beverage are low in phosphorus and the obtained beverages therefore have a phosphorus content much lower than 80 mg/100 g protein. The four beverages are therefore suitable for use as protein beverages for kidney disease patients.

1.-27. (canceled)

28. A packaged, heat-treated beverage preparation having a pH in the range of 2-4.7, the beverage comprising
a total amount of protein of 2 to 45% w/w relative to the weight of the beverage, wherein at least 90% w/w of the protein is beta-lactoglobulin (BLG), and
optionally, sweetener, sugar polymers and/or flavour.

29. The packaged, heat-treated beverage preparation according to claim 28, wherein the preparation is at least pasteurised.

30. The packaged heat-treated beverage preparation according to claim 28, wherein the preparation is sterile.

31. The packaged, heat-treated beverage preparation according to claim 28 wherein the protein fraction of the beverage preparation has an intrinsic tryptophan fluorescence emission ratio (I330 nm/I350 nm) of at least 1.11.

32. The packaged, heat-treated beverage preparation according to claim 28 wherein the protein fraction of the beverage preparation has an intrinsic tryptophan fluorescence emission ratio (I330 nm/I350 nm) of less than 1.11.

33. The packaged, heat-treated beverage preparation according to claim 28, wherein the protein fraction has a degree of protein denaturation of at most 10%.

34. The packaged, heat-treated beverage preparation according to claim 28, wherein the beverage preparation has a degree of protein denaturation of at most 10%.

35. The packaged, heat-treated beverage preparation according to claim 28 having a pH in the range of 3.0-4.3.

36. The packaged, heat-treated beverage preparation according to claim 28 wherein the protein fraction of the beverage preparation has a colour value delta b* in the range of −0.10 to +0.51 at the CIELAB colour scale, wherein delta b*=b_{sample standardized to 6.0 w/w % protein}*−b_{demin. water}*, measured at room temperature.

37. The packaged, heat-treated beverage preparation according to claim 28, wherein the beverage preparation has a colour value delta b* in the range of −0.10 to +0.51 at the CIELAB colour scale, wherein delta b*=b_{sample standardized to 6.0 w/w % protein}*−b_{demin. water}*, measured at room temperature.

38. The packaged, heat-treated beverage preparation according to claim 28, wherein the sum of the amounts of Na, K, Mg and Ca is at most 750 mM.

39. The packaged, heat-treated beverage preparation according to claim 28 having a turbidity of at most 200 NTU.

40. The packaged, heat-treated beverage preparation according to claim 28 having a turbidity of more than 200 NTU.

41. The packaged, heat-treated beverage preparation according to claim 28 having a viscosity of at most 200 cP centipoise, measured at 22 degrees Celsius at a shear rate of 100/s.

42. The packaged, heat-treated beverage preparation according to claim 28 comprising a total amount of protein of 4.0 to 30% w/w relative to the weight of the beverage or preferably 5-20% w/w relative to the weight of the beverage.

43. The packaged, heat-treated beverage preparation according to claim 28 wherein each main non-BLG whey protein is present in a weight percentage relative to total protein which is at most 15% of its weight percentage relative to total protein in a standard whey protein concentrate from sweet whey, more preferably at most 10%, even more preferably at most 6%, most preferably at most 4%.

44. The packaged, heat-treated beverage preparation according to claim 28 wherein at least 92% w/w of the protein is beta-lactoglobulin (BLG).

45. A method of producing a packaged, heat-treated beverage preparation having a pH in the range of 2-4.7, comprising the following steps:
a) Providing a liquid solution comprising:
a total amount of protein of 2 to 45% by weight, wherein at least 90% w/w of the protein is BLG
optionally, sweetener, sugar polymers and/or flavour
b) packaging the liquid solution,
wherein the liquid solution of step a) and/or the packaged liquid solution of step b) is subjected to a heat-treatment comprising at least pasteurisation.

46. The packaged heat-treated beverage preparation according to claim 28, for use in a method for the treatment of diseases associated with protein malabsorption.

47. Use of the packaged heat-treated beverage preparation according to claim 28 as a dietary supplement.

48. Use of the packaged heat-treated beverage preparation according to claim 47, wherein said beverage preparation is ingested before, during or after exercise.
